(19) United States
(12) Patent Application Publication
(10) Pub. No.: US 2017/0080030 A1
(43) Pub. Date: Mar. 23, 2017

(54) COMPOSITIONS AND METHODS FOR TREATING RETINOPATHY

(71) Applicant: University of Virginia Patent Foundation, Charlottesville, VA (US)

(72) Inventors: Shayn Peirce-Cottler, Charlottesville, VA (US); Paul Andrew Yates, Charlottesville, VA (US); Thomas A. Mendel, Charlottesville, VA (US)

(21) Appl. No.: 15/126,460

(22) PCT Filed: Mar. 17, 2015
(86) PCT No.: PCT/US15/20975

§ 371 Date: Sep. 15, 2016

Related U.S. Application Data
(60) Provisional application No. 61/954,111, filed on Mar. 17, 2014.

(51) Int. Cl. A61K 35/28; A61K 38/20; A61K 38/19; A61K 38/18

(52) U.S. Cl. CPC A61K 35/28; A61K 38/1841; A61K 38/1866; A61K 38/1858; A61K 38/1825; A61K 38/204; A61K 38/191; A61K 38/19

(57) ABSTRACT

The present invention encompasses the use of adipose tissue derived cells, or conditioned medium of such cells, to treat and prevent vascular related injuries, diseases, disorders, and conditions such as diabetic retinopathy, where, in one aspect, pericytes such as retinal pericytes have been lost or damaged. The present application further discloses compositions and methods useful for enhancing the function and activity of the adipose tissue derived cells in the treatment and prevention of injuries, diseases, disorders, and conditions including the use of TGFβ to condition the cells to enhance their activity.

### CROSS REFERENCE TO RELATED APPLICATIONS

This application is entitled to priority pursuant to 35 U.S.C. §119(e) to U.S. provisional patent application No. 61/954,111, filed on Mar. 17, 2014. The entire disclosure of the afore-mentioned patent application is incorporated herein by reference.

### STATEMENT REGARDING FEDERALLY SPONSORED RESEARCH OR DEVELOPMENT

This invention was made with government support under Grant Nos. HL082838-02, EY022063-01, and K08 EY019533-02 awarded by The National Institutes of Health. The government has certain rights in the invention.

### BACKGROUND

The therapeutic potential of adipose tissue-derived stem cells (ASCs) has been demonstrated with success in several applications, including myocardial infarction, diabetic wound healing, and neurodegenerative disorders. ASCs are desirable because of their relative ease of harvest from accessible fat depots, as well as their potential for allogeneic or even autologous treatment. However, few studies have examined whether ASCs obtained from diabetic patients are negatively impacted by the disease, pertaining to their role in regenerative medicine.

Studies comparing the efficacy of ASCs from diabetic and non-diabetic sources have focused on their application in diabetic ulcers and hindlimb ischemia. Both of these studies found ASCs derived from diabetic sources to be impaired in their treatment efficacy relative to their non-diabetic counterparts. Studies using stem cells from different sources, such as bone marrow-derived stem cells (BMSCs), have shown functional impairment in cells derived from diabetic mice.

Diabetes profoundly impacts the microvasculature in nearly every tissue. Diabetic retinopathy results in retinal capillary dropout, vessel leakage, and pathological neovascularization, leading to severe and irreversible vision loss. Current surgical and pharmacologic treatments are only effective at managing complications of diabetic retinopathy, but do not repair existing retinal damage. Laser photocoagulation is the current treatment standard for proliferative diabetic retinopathy, and operates on the principle of cauterizing hypoxic retinal tissue. While effective at stemming the progression of retinopathy, this procedure damages peripheral and night vision, often requires repeated treatments, and only prevents visual deterioration in half of cases. Anti-VEGF therapy has been increasingly used alone or in combination with laser therapy, with improvements in vision loss due to diabetic macular edema. However, anti-VEGF therapy requires frequent and painful intra-vitreal injections for several years, and unfortunately does not reverse the underlying pathology. A lasting and non-destructive treatment for diabetic retinopathy is clearly needed.

Pericytes are multifunctional, polymorphic perivascular cells that lie within the microvessel basal lamina and are located on the abluminal side of endothelial cells. Endothelial cells form the inner lining of the vessel wall, and perivascular cells—referred to as pericytes, vascular smooth muscle cells or mural cells—envelop the surface of the vascular tube (Katz et al., U.S. Pat. Pub. No. 2008/0213235, Sep. 4, 2008). Previously, it has been demonstrated that hASCs can differentiate into pericytes and promote microvascular stability and maintenance during angiogenesis in vivo (Amos et al., Stem Cells 26: 2682-2690, 2008; Traktuev D O, et al., Circ Res 102: 77-85, 2008). Yang et al. (2010, Graefes Arch. Clin. Exp. Ophthalmol.) injected human adipose-derived mesenchymal stem cells intravenously into diabetic rats and found that cells which homed to the retina differentiated into photoreceptor-like cells and astrocyte-like cells and that there was increased integrity of the blood brain barrier. Yang found most of the new cells in the retina to be in the outer nuclear layer.

Cells derived from the stromal vascular fraction (SVF) of human adipose tissue have significant developmental plasticity. A fraction of these cells, referred to as human adipose derived stromal cells (hASCs), can be separated from the SVF by their adherence to tissue culture plastic and appear to comprise a homogeneous population based on their similar molecular and cell surface profile.

Data showing that human adipose-derived stromal cells (hASCs), isolated during routine liposuction and excision procedures in a clinical setting, possess the abilities to differentiate into a number of specialized cell types has been validated time and again over the better part of the last decade. hASCs are known to have the capacity to affect their microenvironment in vivo through either differentiation or secretion of relevant growth factors and chemokines.

In diabetic retinopathy, perivascular support cells that would normally stabilize microvessels in the retina and prevent blood leakage are lost from hyperglycemia. The resulting condition is the leading cause of blindness in middle-aged adults in America. Exudative macular degeneration also involves aberrant growth and blood vessels that leak and bleed within the macula leading to loss of central vision. It is the leading cause of vision loss in the elderly. Finally, retinopathy of prematurity affects children born before 31 weeks of gestation and is one of the leading causes of childhood vision loss. In this condition, abnormal blood vessel growth and associated fibrovascular proliferation can ultimately lead to retinal detachment and blindness.

A second known class of pluripotent cells, bone marrow derived stem cells, have previously been found to be useful for treating a number of ocular diseases. Bone marrow-derived stem cells derive from a different cellular compartment and are phenotypically and functionally distinct from hASCs. Bone marrow-derived stem cells, injected intravitreally or within the orbital sinus are capable of repairing retinal vascular damage. Both streptozotocin-induced diabetic mice and neonatal mice with oxygen induced retinopathy (OIR) injected intravitreally and systemically with healthy human derived CD34+ endothelial precursor cells had these cells attach and assimilate into the damaged vasculature. This repair involves reendothelialization of acellular vessels by the CD34+ cells. In OIR mice, the CD34+ cells were injected after hyperoxic injury and homed to the site of vascular damage.

A separate study showed that intravitreally injected human bone marrow-derived LIN—hematopoietic stem cells (LHSCs) did not prevent vasoobliteration from hyperoxia, but did accelerate vascular repair once the OIR mice were returned to normoxia. Intravitreally injected LHSCs have also been shown to prevent vascular degeneration in the rd1 and rd10 mouse models of retinal degeneration. This effect appears to be secondary to upregulation of anti-apoptotic genes. LHSCs are known to function as hemangioblasts during retinal neovascularization and can contribute to laser induced choroidal neovascularization. The mechanism through which they promote vessel growth is unclear and may involve either a paracrine effect or may be secondary to the perivascular location of some of these cells as retinal microglia.

There is a long felt need in the art for compositions and methods useful for treating eye pathologies. The present invention satisfies this need.

### SUMMARY OF THE INVENTION

Diabetic retinopathy is a debilitating disease that leads to progressive retinal vascular pathologies and ultimately to vision loss which current treatments are unable to reverse. Intra-vitreal injection of adipose-derived stem cells (ASCs) stabilizes retinal microvasculature and encourages regeneration of damaged capillary beds in several mouse models of retinal vasculopathy. ASCs are advantageous because of their relative ease of harvest from accessible fat depots, as well as their potential for autologous or allogeneic treatment. Understanding the status of ASCs harvested from diabetic patients is of critical importance for moving forward with autologous therapies.

Using the hyperglycemic Akimba mouse model of diabetic retinopathy, the differences in treatment efficacy and function of murine adipose-derived stem cells (mASCs) derived from healthy wildtype vs. diabetic Akimba mice were examined herein (see Example 1). It is disclosed herein that hyperglycemia impairs the regenerative ability of adipose-derived stem cells in the treatment of diabetic retinopathy. Akimba mice received intra-vitreal injections of mASCs. mASCs from healthy, non-diabetic mice were more effective than diabetic mASCs in revascularizing the diabetic retina and in protecting against vessel loss. Vessel drop-out is one of the hallmarks of this pathology and it is disclosed herein that healthy ASCs are better in protecting against vessel drop-out than ASCs derived from a diabetic. Additionally, a greater number of healthy mASCs were found incorporated into the retina than diabetic mASCs. mASC viability was assessed using TUNEL and EdU incorporation assays, which revealed that healthy mASCs proliferate more rapidly and undergo less apoptosis than diabetic mASCs. When compared to diabetic mASCs, healthy mASCs secreted more angiogenesis-promoting factors, such as IGFBP-2/3 and MCP, as determined by high-throughput ELISA.

It is disclosed herein (see Example 1) that mASCs from healthy, non-diabetic mice are more effective than diabetic mASCs from Akimba mice in revascularizing and stabilizing the diabetic retina by protecting against vessel drop-out. The present invention therefore encompasses the use of ASCs to enhance vascularization, to increase vascular density, and to protect against vessel drop-out. To provide a mechanism for this observed difference in regenerative ability, a functional analysis of healthy and diabetic mASC was carried out. It is disclosed herein that several pro-angiogenic factors, including some known to effect retinal angiogenesis, are secreted at significantly higher levels in healthy versus diabetic mASCs. In one aspect, the invention encompasses the use of an effective amount of one or more of IGFBP-2, IGFBP-3, MCP-1, osteopontin, and SDF-1 to treat a subject in need thereof, and the invention encompasses the use of one or more of these factors in conjunction with other treatments such as the administration of ASCs.

One of ordinary skill in the art will appreciate that additional growth factors and chemokines can be used as well, and that that any or all of the factors can be used in combination with autologous cells to help improve incorporation of the cells into the retina, increase the rates of proliferation, and lower the rate of apoptosis in the autologous ASCs. In one aspect, the ASCs are pretreated with TGF-β prior to administration or are administered simultaneously with an effective amount of TGF-β. The present invention further encompasses the use of conditioned medium derived from ASCs to treat damaged retinal vasculature. In one aspect, the conditioned medium is serum-free conditioned medium. In one aspect, the cells are human.

Because diabetic mASCs exhibited lower rates of proliferation and greater rates of apoptosis than healthy mASCs (see Example 1), in one embodiment, the results do not support the use of autologous ASCs from diabetic patients if normal cells are available or unless the cells are pretreated to enhance their ability to stimulate revascularization or other factors are also administered. The data suggest that use of an “allograft” or “allogeneic” ASC cells will enhance the effect and in one embodiment the invention encompasses the use of such cells in the treatment. One of ordinary skill in the art will know which match is needed or when the best match will be needed.

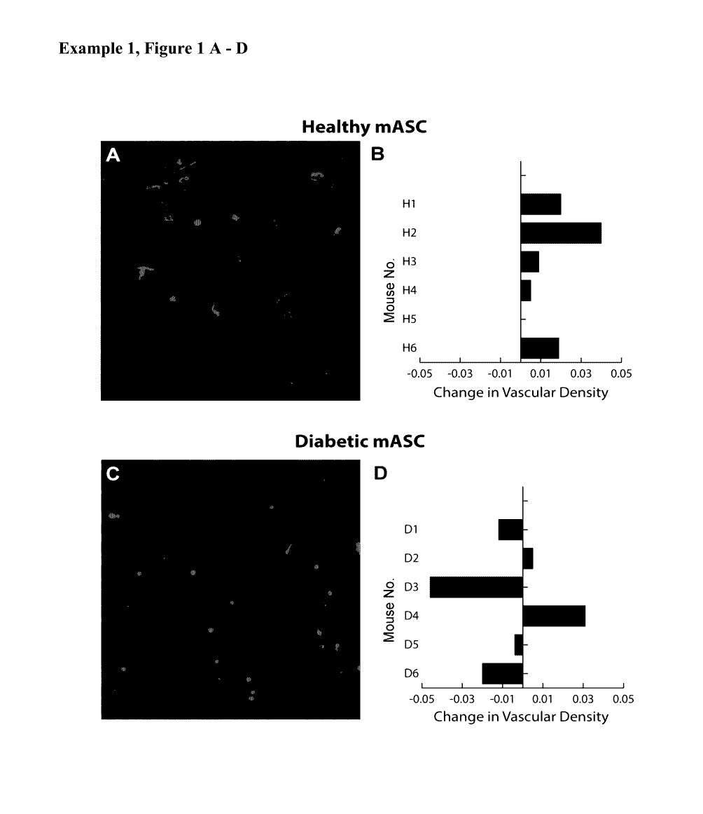

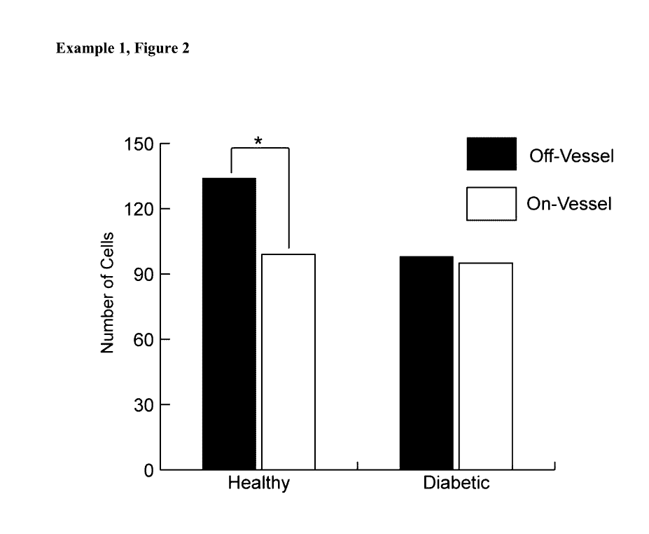

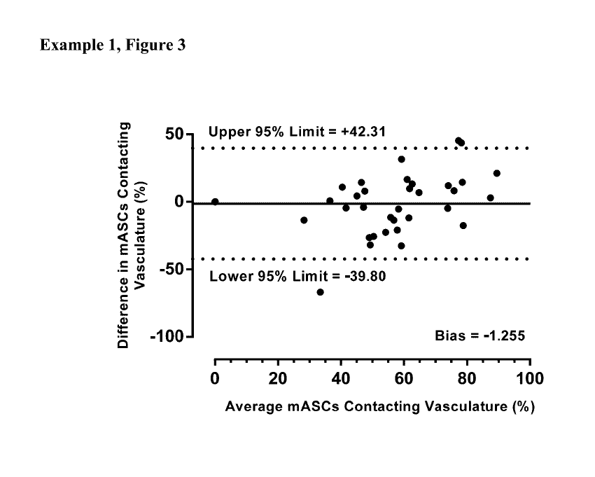

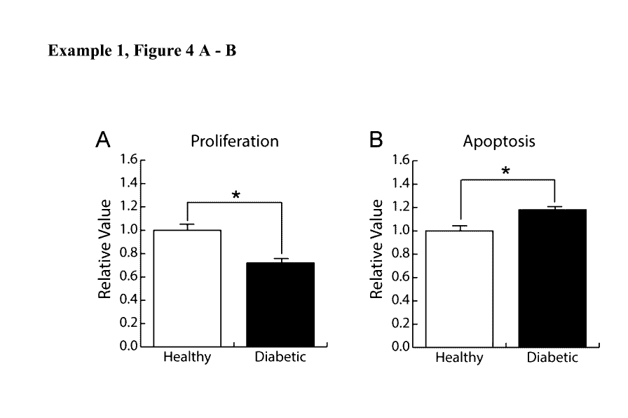

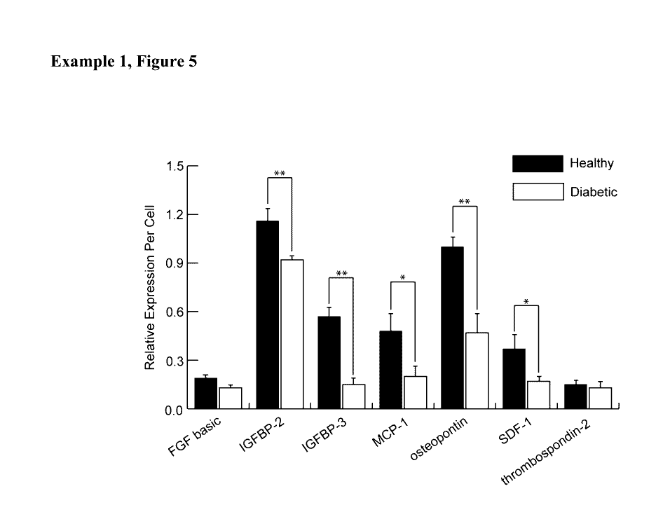

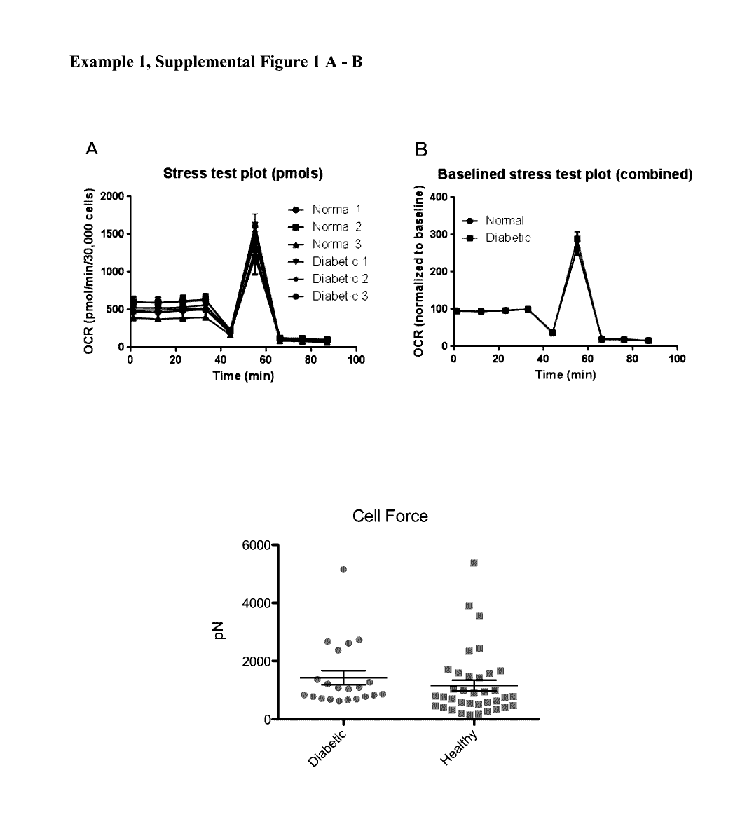

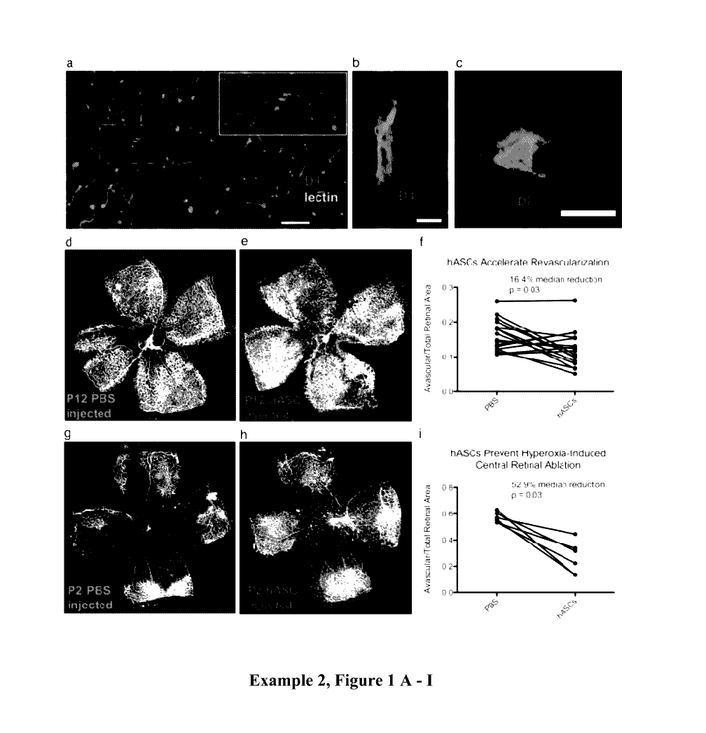

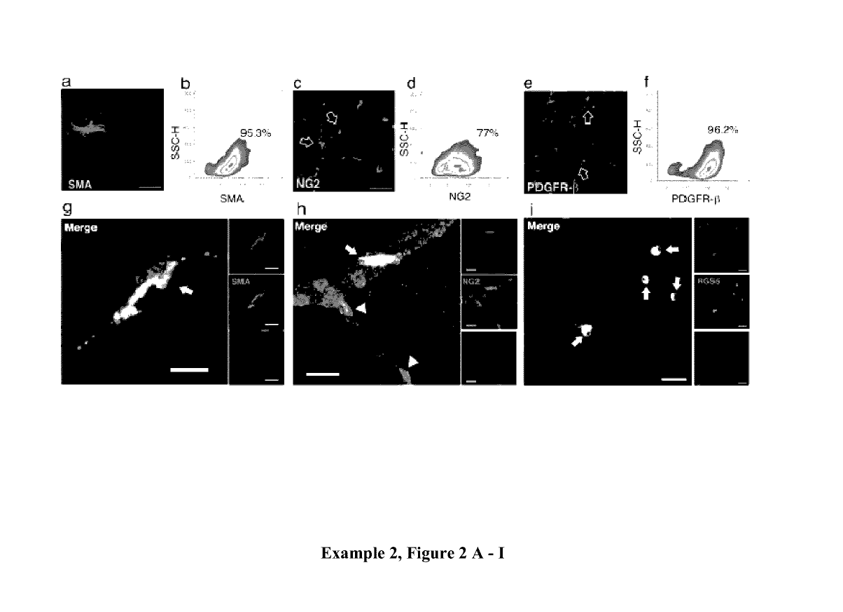

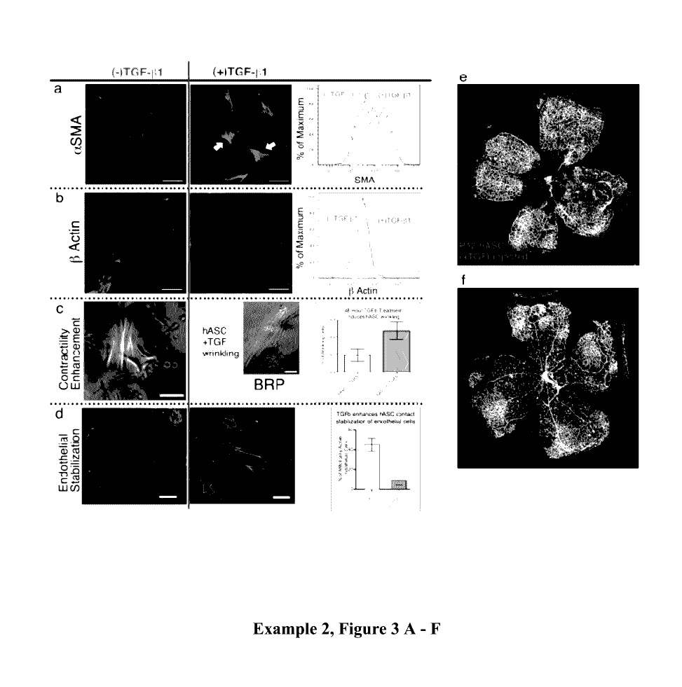

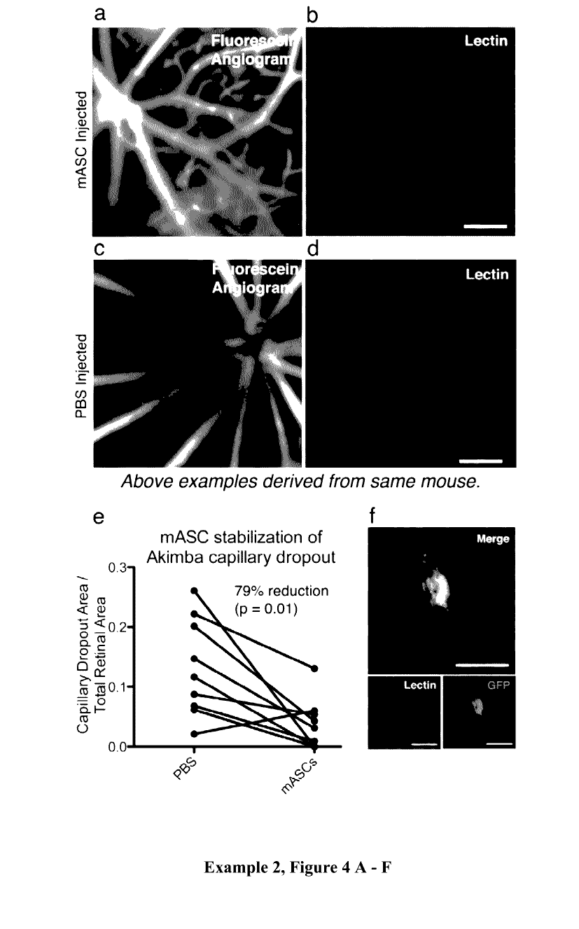

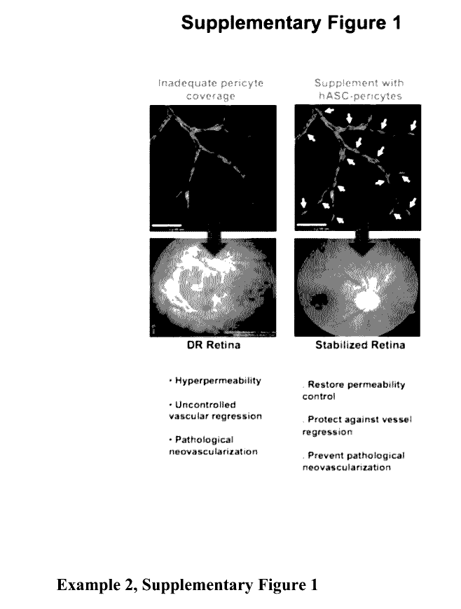

It is disclosed in Example 2 that human ASCs also incorporate into the retina, including into the mouse retina, and that these cells can induce improvement of revascularization in a damaged retina, including in the central retina. It is also disclosed herein that the ASCs exhibit pericyte phenotypic markers in vitro and maintain this expression in vivo and the exposure of the cells to TGF-β prior to administration increases expression of pericyte markers. The present invention therefore encompasses the use of TGF-β pretreatment of ASCs to inhibit or prevent long term retinal capillary dropout in diabetic retinopathy (see also, Example 2, Supplementary FIG. 1). I

In one embodiment, ASCs are injected intravitreally. In one embodiment, ASCs are injected subretinally,

It is disclosed herein that injected ASCs act as vascular support cells (see Example 3), that pericytes from ASCs can invade a laser induced scar, that TGF-β pretreatment (preconditioning) of ASCs reduces proliferation of co-cultured endothelial cells, that ASCs can prevent or treat hyperoxia-induced retinal ablation, that ASCs decrease retinal capillary dropout, that healthy ASCs have greater proliferative capacity and demonstrate less apoptosis than ASCs from diabetics, that healthy ASCs secrete more of certain angiogenic factors than ASCs from diabetic subjects, and that pretreatment of ASCs with TGF-β prior to administration to a diabetic subject enhances hyperoxic endothelial cell stabilization.

The present invention further encompasses compositions and methods useful for encapsulating cells prior to administration (see Example 3). This method allows for choosing an encapsulation system that either releases cells or allows the cells to secrete their factors that stimulate angiogenesis, etc. and for the secretions to diffuse through the encapsulation materials to the part of the retina that is being targeted. The cells can be pretreated prior to administration and other factors and compounds can be included with the cells before administration to the subject.

In one embodiment, the compositions and methods of the invention are useful for treating various diseases, disorders, and injuries. In one aspect, the disease, disorder, or injury includes, but is not limited to, diabetic retinopathy, retinopathy, arteriosclerotic retinopathy, hypertensive retinopathy, proliferative vitreoretinopathy, retinal tears, retinal detachment, macular degeneration, age related macular degeneration, inflammatory retinal disease, retinal vasculitis, retinal fibrosis, diffuse unilateral subacute neuroretinitis, cytomegalovirus retinitis, Stargardts, Best's Disease, Usher Syndrome, papilloedema, surgery, surgical/treatment side effect, vitelliform maculopathy, retinitis pigmentosa, cone-rod dystrophy, retinal separation, retinal hypoxia, aberrant neovascularization of the retina, retinal scar formation, and retinoblastoma. In one aspect, the disease, disorder, or injury is diabetic retinopathy. In one aspect, the method prevents or inhibits the formation of subretinal scar tissue. In one aspect, the method enhances retinal regeneration or retinal repair. In one aspect, the method inhibits proliferative vitreoretinopathy. In one embodiment of the invention, the method can be performed during open-globe or retinal detachment repair.

In one embodiment, the compositions and methods of the invention are useful for stimulating revascularization in the diabetic retina. In one embodiment, the compositions and methods of the invention are useful for stabilizing vasculature in the diabetic retina. In one embodiment, the compositions and methods of the invention are useful for enhancing hyperoxic endothelial cell stabilization. In one embodiment, the compositions and methods of the invention are useful for increasing vascular length density.

In one embodiment, the present invention further encompasses the use of combination therapies and treatments. For example, when adipose-derived stem cells are administered to a subject, that treatment can be performed in combination with administration of adipose-derived stem cell-conditioned medium. Additionally, encapsulated adipose-derived stem cells can be administered. In one aspect, additional growth factors, cytokines, pro-angiogenic factors, and additional therapeutic agents can be used in combination with one or more treatments of the invention.

The compositions, cells, and methods of the invention are useful for treating other diseases and disorders where retinal microvasculature has been damaged or where there is less microvasculature than is typically present in a normal individual.

The present invention provides compositions and methods for obtaining adipose tissue, for isolating adipose-derived stem cells, for purifying adipose-derived stem cells, and for administering adipose-derived stem cells. Furthermore, techniques useful for these purposes are also known in the art.

In one embodiment, the present invention provides compositions and methods for preventing or treating a retinal disease, disorder, or injury. In one aspect, the method comprises administering to a subject in need thereof a pharmaceutical composition comprising an effective amount of adipose-derived stem cells. In one aspect, the composition optionally comprises a pharmaceutically-acceptable carrier. In one aspect, the composition optionally comprises an additional therapeutic agent. In one aspect, the composition further comprises at least one growth factor, cytokine, or angiogenic factor. In one aspect, the composition further comprises adipose-derived stem cell-conditioned medium.

In one aspect, the adipose-derived stem cells are capable of differentiating into pericytes.

In one aspect, the cells are injected intravitreally or subretinally.

In one aspect, the administered cells integrate into retinal vasculature. In one aspect, at least one of the administered cells expresses pericyte markers. In one aspect, at least one of the cells differentiates into a pericyte.

In one embodiment, TGF-β can be administered with the cells or injected intravitreally or subretinally before cells are administered. In another aspect, TGF-β is administered after the cells are injected. The amount of TGF-β used will vary depending on whether cells are pretreated or whether the TGF-β is administered to the subject. One of ordinary skill in the art can easily determine how much to use.

In one embodiment, the adipose-derived stem cells administered to a subject are pretreated with transforming growth factor β (TGFβ) prior to administration. In one aspect, the TGFβ is used at a concentration of about 0.1 ng/ml to about 10 ng/ml. In another aspect, the TGFβ is used at a concentration of about 1.0 ng/ml. In one aspect, pretreatment with TGFβ enhances the pericyte phenotype of the treated adipose-derived stem cells. In one aspect, TGFβ is also administered to the subject after the cells are administered.

In one embodiment, TGFβ is administered to a subject after untreated (no TGFβ pretreatment) adipose-derived stem cells are administered.

In one aspect, TGFβ enhances the pericyte phenotype of the treated adipose-derived stem cells.

The present invention further provides for the use of VEGF 165b as a treatment for the retina diseases and disorders discussed herein such as diabetic retinopathy. In one aspect, VEGF 165b is administered in conjunction with the administration of ASCs. In one embodiment, vascular endothelial growth factor 165b (VEGF 165b) is administered after the cells are administered. In one embodiment, VEGF 165b and the cells are administered at the same time.

In one embodiment, a method of the invention prevents or inhibits retinal capillary dropout.

In one embodiment, the cells administered to a subject are autologous cells.

The present invention further provides methods for treating retinal injury and diseases comprising administering ASCs to increase vascular length density in the retina.

In one embodiment, the composition comprises an effective amount of at least one angiogenic factor. In one aspect, the angiogenic factor is selected from the group consisting of IGFBP-2, IGFBP-3, MCP-1, osteopontin, and SDF-1. In one aspect, the composition comprises an effective amount of at least one growth factor or cytokine. In one aspect, the growth factor or cytokine is selected from the group consisting of VEGF, (platelet-derived growth factor) PDGF, fibroblast growth factor (FGF), tumor necrosis factor α (TNFα), interleukin-6 (IL-6), and endothelin-1.

In one aspect, a method of the invention stimulates vascularization of the retina. In one aspect, the method increases vascularization. In one aspect, vascularization is in the retina. In one aspect, the method stabilizes preretinal neovascularization. In one aspect, the method increases vascular length density. The methods of the invention are useful for enhancing recovery from retinopathy.

One of ordinary skill in the art will know that the number of cells to be administered, as well as whether cells should be administered more than once, will depend on factors such as the source of the cells and the age, sex, health, and weight of the subject, as well as the particular disease, disorder, or injury being treated. Cells can be administered as a unit dose, as a number per unit volume, or per unit weight of the subject (see, for example, Siquiera et al., 2011, Retina, 31:1207-1214). Ranges of cells to be administered include, about 10,000 to about 100,000,000, about 50,000 to about 50,000,000, about 100,000 to about 10,000,000, and about 1,000,000 to about 8,000,000. The numbers of cells include numbers within the ranges, including, but not limited to, about, 10,000, 25,000, 50,000, 75,000, 100,000, 200,000, 300,000, 500,000, 750,000, 1,000,000, 1,250,000, 1,500,000, 1,750,000, 2,000,000, 2,250,000, 2,500,000, 2,275,000, 3,000,000, 3,250,000, 3,500,000, 3,750,000, 4,000,000, 4,250,000, 4,500,000, 4,750,000, 5,000,000, 5,250,000, 5,500,000, 5,750,000, 6,000,000, 6,250,000, 6,500,000, 6,750,000, 7,000,000, 7,250,000, 7,500,000, 7,750,000, 8,000,000, 8,250,000, 8,500,000, 8,750,000, 9,000,000, 9,250,000, 9,500,000, 9,750,000, 10,000,000, 15,000,000, 25,000,000, and 75,000,000.

One of ordinary skill in the art can also determine the volume of the pharmaceutical composition to be administered and how it can be varied according to such things as the number of cells being administered. Volumes can include, for example, a range of about 0.01 ml to about 1.0 ml, about 0.05 ml to about 0.08 ml, and about 0.1 ml to about 0.5 ml. The volumes include individual volumes within that range, including, for example, 0.01 ml, 0.02, ml, 0.03, ml, 0.04 ml, 0.05 ml, 0.06 ml, 0.07 ml, 0.08 ml, 0.09 ml, 0.1 ml, 0.15 ml, 0.2 ml, 0.25 ml, 0.3 ml, 0.35 ml, 0.4 ml, 0.45 ml, 0.5 ml, 0.6 ml, 0.7 ml, 0.8 ml, 0.9 ml, and 1.0 ml.

In one embodiment, the cells used in the invention are human cells. In one aspect, the subject is human.

In one embodiment, one or more growth factors, cytokines, or angiogenic factors are also administered to a subject in need thereof who is being treated with adipose-derived stem cells and/or adipose-derived stem cell-conditioned medium. For example, when VEGF 165b is administered to a subject, other factors can be administered. These can include other VEGFs, PDGF, FGF, TNFα, IL-6, and endothelin-1, and biologically active fragments and homologs thereof. In one aspect, at least one angiogenic factor is also administered. Angiogenic factors include, but are not limited to, IGFBP-2, IGFBP-3, MCP-1, osteopontin, and SDF-1. In one aspect, adipose-derived stem cell-conditioned medium is administered to the subject as well. One of ordinary skill in the art, based in the teachings provided herein, will appreciate that various combinations of adipose-derived stem cells, adipose-derived stem cell-conditioned medium, growth factors, cytokines, and angiogenic factors can be administered to a subject in need. As disclosed herein, the administration of at least one growth factor, cytokine, or angiogenic can be in conjunction with administration of adipose-derived stem cells and/or adipose-derived stem cell-conditioned medium.

In one embodiment of the invention, when a subject is being treated at least one additional therapeutic agent is administered. These agents include, but are not limited to, antimicrobials, antibiotics, hormones, hormone antagonists, chemokines, steroids, pain medications, non-steroidal anti-inflammatory agents, anesthetics, anti-inflammatory agents, and combinations thereof.

In one embodiment, adipose-derived stem cell-conditioned medium is administered to a subject in need thereof. In one aspect, the conditioned medium is administered to a subject who also receives adipose-derived stem cells. In one aspect, the adipose-derived stem cell-conditioned medium is cell-free. In one aspect, the adipose-derived stem cell-conditioned medium is serum-free. In one aspect, the conditioned medium comprises at least one of insulin-like growth factor binding protein-2 (IGFBP-2), IGFBP-3, monocyte chemoattractant protein (MCP-1), osteopontin, and stromal cell-derived factor-1 (SDF-1). In one aspect, the conditioned medium comprises each of IGFBP-2, IGFBP-3, MCP-1, osteopontin, and SDF-1. In one aspect, the conditioned medium comprises an effective amount of at least one of IGFBP-2, IGFBP-3, MCP-1, osteopontin, and SDF-1.

In one aspect, the conditioned medium is supplemented with at least one factor disclosed herein or other useful growth factors, cytokines, or angiogenic factors. In one aspect, the factors include, but are not limited to vascular endothelial growth factor 165b (VEGF165b), transforming growth factor β (TGFβ), VEGF, (platelet-derived growth factor) PDGF, fibroblast growth factor (FGF), tumor necrosis factor α (TNFα), interleukin-6 (IL-6), and endothelin-1. In one aspect, the cells are derived from the subject.

In one aspect, adipose-derived stem cell-conditioned medium of the invention can be used for treating the same diseases, disorders, and injuries as those treated with adipose-derived stem cells. In one aspect, the results of the treatment with conditioned medium are the same as when administering the cells of the invention.

In one embodiment, the present invention provides compositions and methods for preventing or treating a retinal disease, disorder, or injury comprising administering to a subject in need thereof an effective amount of encapsulated adipose-derived stem cells. In one aspect, the encapsulated cells are administered during open-globe or retinal detachment repair. In one aspect, the cells are encapsulated in alginate based microbeads. In one aspect, the encapsulated cells are administered subretinally or intravitreally. In one aspect, the method prevents or inhibits the formation of subretinal scar tissue. In one aspect, the cells are allogeneic. In one aspect, the method enhances retinal regeneration or retinal repair. In one aspect, the method inhibits proliferative vitreoretinopathy. In one aspect, the cells remain viable for at least about one week, or at least about two weeks, or at least about one month. In one aspect, the cells remain viable for at least about four months or at least about six months. In one aspect, conditioned medium prepared from the encapsulated cells is administered to the subject. In one aspect, the encapsulated cells are pretreated with TGFβ prior to administration to a subject. The method is useful for treating the diseases, disorders, and injuries, described herein.

The present invention further provides a kit useful for practicing the methods of the invention. The kit can include, for example, adipose-derived stem cells, one or more growth factors, one of more regulators of angiogenesis, adipose-derived stem cell-conditioned medium, one or more additional therapeutic agents, encapsulation materials, and an instructional material for the use thereof.

Various aspects and embodiments of the invention are described in further detail below.

### BRIEF DESCRIPTION OF THE DRAWINGS

Figures are numbered consecutively herein according to the Example (1-3) in which they are cited and the term “Example” is used for each figure and for each reference to a figure in the Brief Description of the Drawings and in the Examples themselves. The Examples utilize the same numbering and the Brief Description of the Drawings is numbered accordingly. Example 1 comprises 6 figures total, including five figures labeled “Example 1”, FIGS. 1-5 and one labeled “Example 1, Suppl.” FIG. 1. Example 2 comprises 8 figures total, including four labeled “Example 2”, FIGS. 1-4 and four labeled “Example 2, Supp.” FIGS. 1-4. Example 3 comprises 9 figures and they are labeled “Example 3” FIGS. 1-9. There are a total of 23 figures corresponding to the numbering used herein.

Example 1 Figures

Example 1, FIG. 1. Treatment with healthy and diabetic mASCs have differential effects on the retinal microvasculature. 5-week old Akimba mice received intraocular injections of “healthy” or “diabetic” passage-4, DiI-labeled mASCs in the right eye and contralateral phosphate-buffered saline control injections in the left eye. 10× confocal microscopy images with mASCs in red and lectin-stained vessels in blue of healthy cell-treated (A) and diabetic cell-treated (C) retinae. Differences in vascular densities for each mouse between cell-treated retinae and contralateral PBS controls showed no change in diabetic cell-treated (B), and a significant increase in healthy cell-treated (D), n=6, p<0.05, paired t-test.

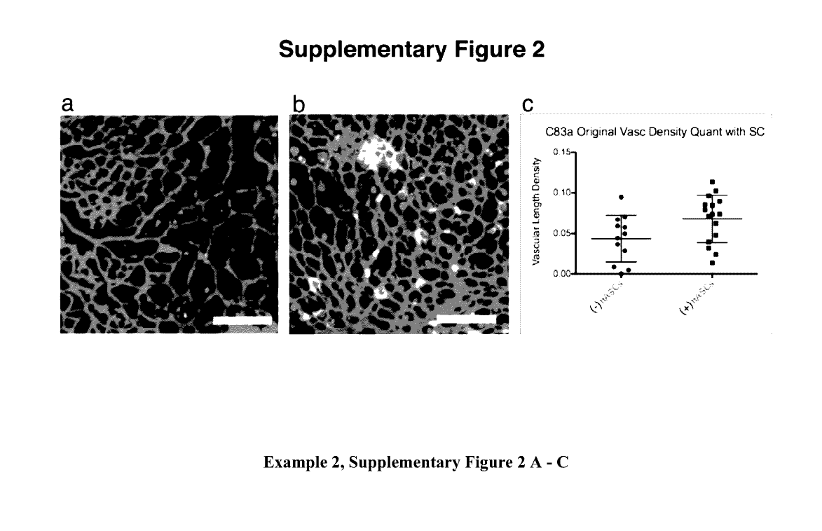

Example 1, FIG. 2: Healthy mASCs incorporate in the retina in greater numbers and assume perivascular positions more often than diabetic mASCs. Cells occupying perivascular and non-perivascular positions counted in representative 20× confocal image stacks. Cell numbers averaged within each animal, then added. n=6, *p<0.05, Mann-Whitney Rank Sum test.

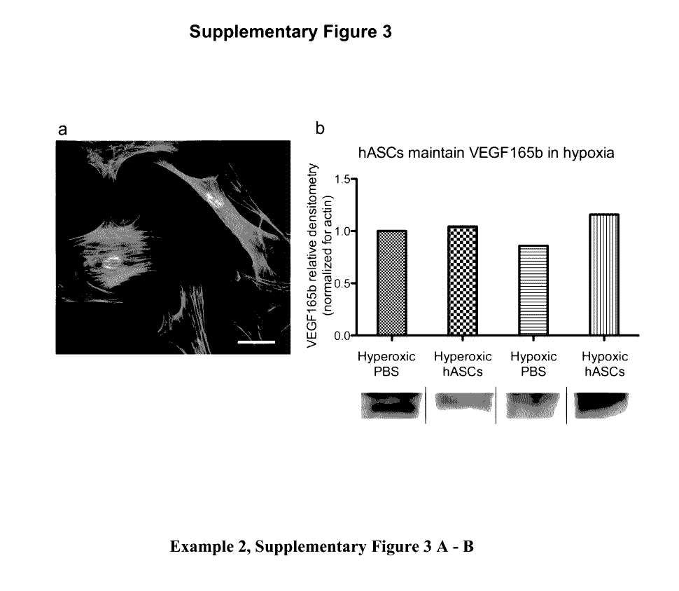

Example 1, FIG. 3: Bland-Altman analysis demonstrates that there is no difference between blind counting and the Monte Carlo simulation in measuring the percent of mASCs contacting retinal vasculature. The difference in measurements of the two methods is plotted as a function of the average in the measurements of the two methods for each sample. The dotted lines represent the 95% upper and lower limits of agreement. The 95% upper and lower limits of agreement are −39.80% and 42.31%, respectively. The solid line indicates an average bias of −1.255%.

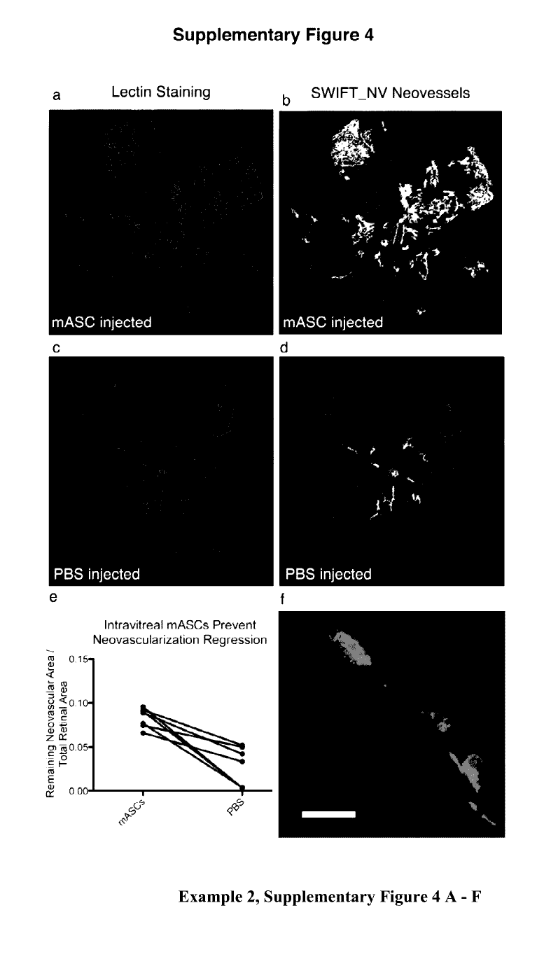

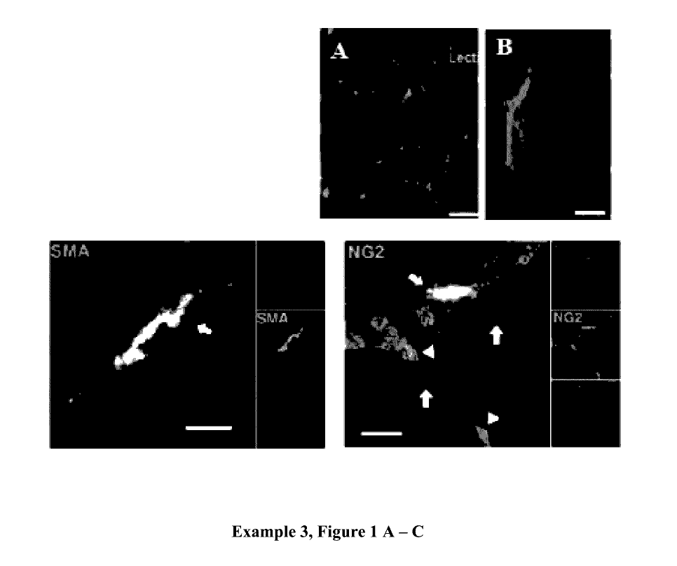

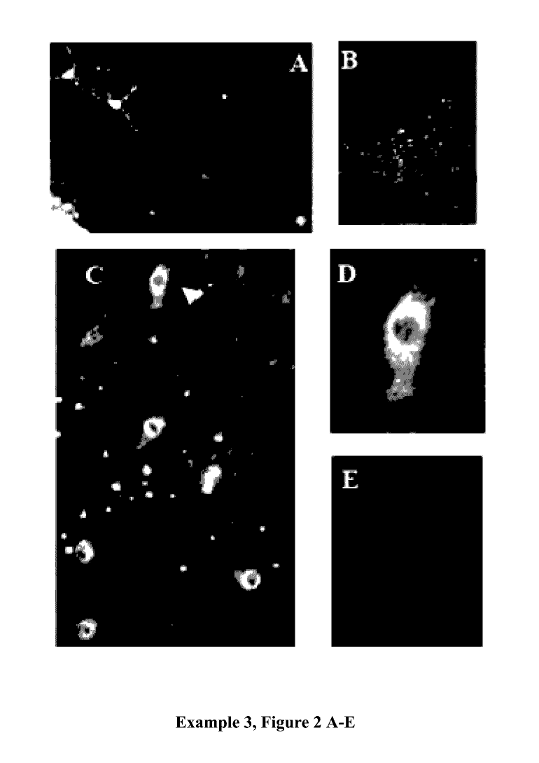

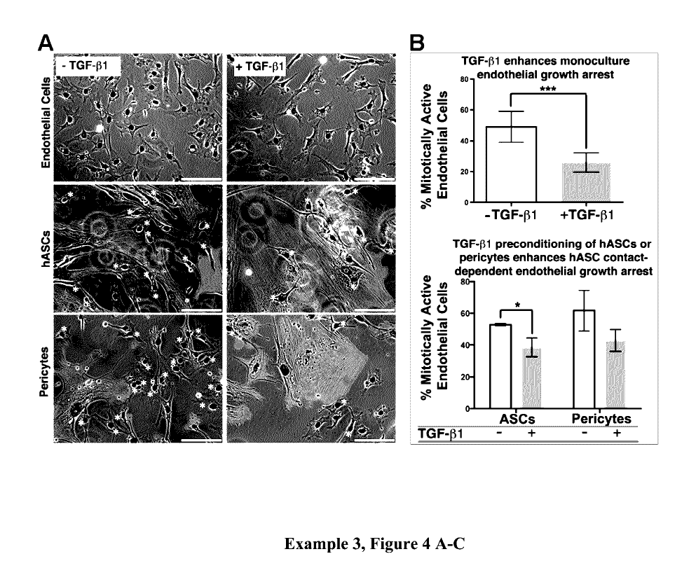

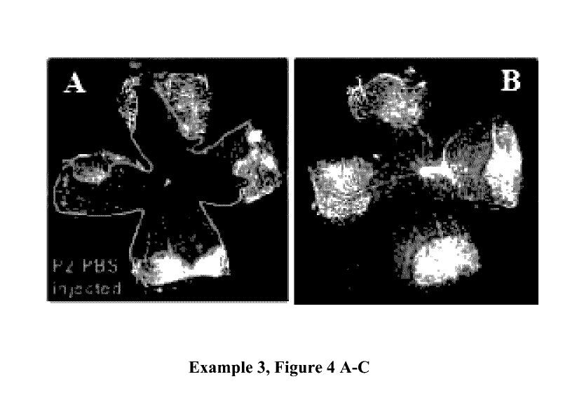

Example 1, FIG. 4: Healthy mASCs exhibit higher rates of proliferation and lower rates of apoptosis than diabetic mASCs. A. Relative number of cells in S phase determined by EdU incorporation assay. B. Cells undergoing apoptosis determined by TUNEL assay. mASCs from 3 healthy and 3 diabetic animals were used for each condition. Values were normalized to the healthy value in each case. *p<0.001, t-test.

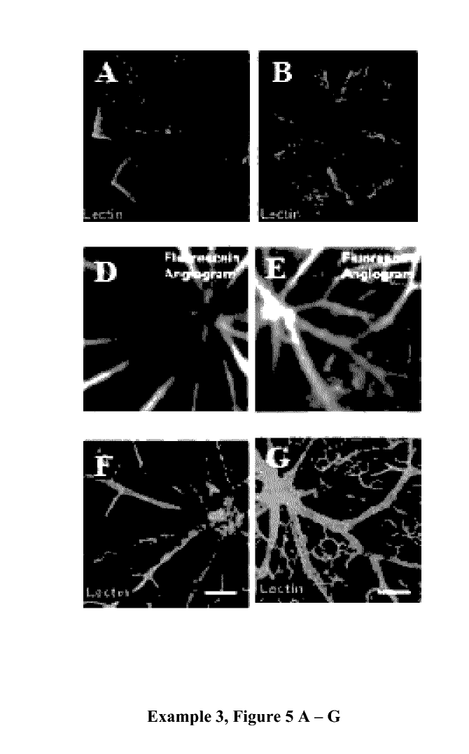

Example 1, FIG. 5. Diabetic mASCs secrete lower levels of angiogenic factors. Relative expression levels of secreted angiogenesis factor of healthy versus diabetic mASCs were taken using high-throughput ELISA arrays. mASCs were isolated from “healthy” wild-type and “diabetic” Akimba mice and maintained to passage 4. Samples consisted of conditioned media obtained by incubating cells immediately after passage in fresh media, and collecting media after 24 hours. n=3, *p<0.05, **p<0.01, t-test

Example 1, Supplementary FIG. 1: Healthy and diabetic mASCs show no difference in metabolic activity. Healthy and diabetic mASCs were isolated from three diabetic mice and three healthy mice, passaged to P4, and subjected to mitochondrial stress tests on the Seahorse XF Bioanalyzer. Individual profiles (A) and combined profiles (B) show no difference in baseline cellular respiration, spare respiratory capacity, ATP-linked respiration, uncoupled respiration, or non-mitochondrial respiration. Total cell force of diabetic and healthy mASCs is not significantly different. Data displayed are Mean±SE, N=21 Diabetic Cells, N=36 Healthy Cells.

Example 2 Figures

Example 2, FIG. 1: Intravitreally injected human adipose derived stem cells (hASCs) home to and stabilize murine retinal microvasculature. 1a, DiI labeled (red) hASCs injected intravitreally at postnatal day 12 (P12) following oxygen induced retinopathy (OIR) home from the vitreous to murine retinal microvasculature (green) as seen following harvest at P17. 1b, c, DiI labeled hASCs (red) wrap around isolectin labeled retinal microvessels (blue) abluminally and target vascular junctions, both properties of terminally differentiated pericytes. d-f, Eyes of NOD SCID mice, injected intravitreally with hASCs (red) at P12 following OIR, and then harvested at P14, demonstrate improved revascularization of central retina as compared to contralateral PBS injected (blue) carrier controls (16.4% reduction, p=0.03). g-i, NOD SCID eyes injected with intravitreal hASCs (red) at P2, prior to OIR, exhibited dramatically less central vascular ablation at P12 than contralateral PBS injected carrier controls (blue) (52.9% reduction, p=0.03). Lines in f and i connect data points for hASC injected and contralateral PBS injected eyes in the same mouse. Scale bars a=200 μm; b, c=20 μm.

Example 2, FIG. 2: hASCs exhibit pericyte phenotypic marker expression in vitro and maintain this expression at late time point's in vivo following integration with the retinal microvasculature. a-f, Passage 5 hASCs exhibit in vitro expression of the characteristic pericyte markers smooth muscle actin (SMA), NG2 chondroitin sulphate proteoglycan (NG2), and platelet derived growth factor receptor beta (PDGFR-β) by both immunohistochemical staining on cultured cells (a,c,e) (yellow arrows) and by flow cytometry on cells harvested from these cultures (b,d,f). g-i, DiI labeled hASCs injected intravitreally into NOD SCID mice at P12, after OIR hyperoxia, are found still associated with the retinal microvasculature 6-8 weeks later and demonstrate persistent SMA, NG2, and regulator of G-protein signaling 5 (RGS5) marker staining (yellow arrows). Note native retinal pericytes are also labeled with NG2 but lacking DiI staining (white arrowheads). Scale bars: a, c, e=100 μm; g=10 h=20 μm, i=48 μm.

Example 2, FIG. 3: TGFβ conditioning of cultured hASCs enhances their pericyte phenotype in vitro and in vivo. a, b, 48 hour pre-conditioning of cultured hASCs with 1 ng/ml of TGFβ increases their expression of the pericyte marker smooth muscle actin (SMA) in culture, as measured by both immunohistochemistry and flow cytometry, while their expression of β Actin control remains unchanged. c, pre-conditioning with TGFβ also enhances the ability of hASCs to contract and deform a silicone substrate (white arrowheads) by 2.6 fold, a response similar to that seen with bovine retinal pericytes. (p=0.0413). d, TGFβ conditioned hASCs (green) when co-cultured with (bovine, human) endothelial cells (blue and pink—yellow arrowheads) decrease the number of endothelial cells still undergoing mitosis (blue) while contacting hASCs by 78%. Stable and quiescent endothelial cells (pink) are seen in far greater numbers in cultures containing TGFB conditioned hASCs as opposed to unconditioned hASCs e, Eyes intravitreally injected with TGFβ preconditioned hASCs at postnatal day 5 (P5), demonstrated an 11.1% (n=, p=0.01) median reduction in retinal avascular area upon removal from hyperoxia at P12, as compared to contralateral control eyes injected with unconditioned hASCs (f). Scale bars: a, b=100 μm; c, d=49 μm.

Example 2, FIG. 4: TGFβ conditioned mASCs prevent long-term retinal capillary dropout in diabetic retinopathic Akimba mouse. a-d, TGFB conditioned mASCs were injected at postnatal day 9 (P9) in Akimba mice, with PBS carrier control injected in the contralateral eye. Retinal capillary loss in 8 week old Akimba mice was substantially decreased in eyes injected with mASCs (a,b) as compared to contralateral eyes injected with PBS carrier control (c,d). Preservation of retinal capillaries is revealed by both fluorescein angiography (a,c) and post-harvest lectin stained retinal whole mounts (b,d) with all panels in a-d taken from the same mouse. e, TGFB conditioned mASCs dramatically reduced the area of capillary dropout as compared to contralateral PBS injected controls (79% reduction, p=0.1). Lines connect data points for mASC and contralateral PBS injected eyes from the same mouse. Scale bars: b, d=150 μm; =75 μm.

Example 2, Supplementary FIG. 1: Proposed cellular treatment for diabetic retinopathy using human adipose derived stem cells (hASCs) to potentially prevent progression of retinal disease and enhance regeneration of the microvasculature damaged by uncontrolled diabetes. a, Retinal microvessels have the highest pericyte (green) to endothelial cell (red) ratio of any vascular bed in the body. These pericytes both protect and support retinal endothelial cells through direct contact and paracrine mechanisms, to help maintain a healthy retinal capillary network (arrow). b. Chronic diabetic inflammation causes eventual loss of native retinal pericytes leading to destabilization of the retinal microvasculature. Acellular capillaries with retinal capillary dropout (arrow) result, soon followed by augmented vascular endothelial growth factor (VEGF) expression, macular edema, retinal hemorrhage, fibrosis, with tractional retinal detachment, and eventually blindness. c, Intravitreally injected hASCs derived pericytes will migrate to and integrate with the retinal microvasculature to augment the remaining native retinal pericyte population. hASC derived pericytes will help stabilize the vascular bed to prevent progression of disease by both direct association with the retinal capillary bed and by paracrine conditioning of the retinal microenvironment to enhance regeneration of lost capillaries.

Example 2, Supplementary FIG. 2: Retinal microvasculature with local hASC integration demonstrates increased vascular length density. DiI labeled hASCs (red) were injected into the vitreous of postnatal day 2 (P2) NOD SCID mice, with the mice exposed to oxygen-induced retinopathy (OIR) from P7 to P12, and retinas harvested at P12. With only the DiI channel visible on the retinal wholemount, retinal sub-fields were selected by a blinded investigator in equal numbers either containing or not containing labeled hASCs. The image of the lectin labeled vasculature (green) was then overlayed on these selected subfields, and the retinal vascular length density of these areas was then determined. Microvasculature associated with locally integrated hASCs (b) demonstrated a significant increase in vascular length density as compared to sub-fields of microvasculature without hASC integration (a) (p=0.031).

Example 2, Supplementary FIG. 3: VEGF 165b is expressed in cultured hASCs and higher levels of VEGF165b are seen in hASC injected eye post-oxygen induced retinopathy exposure a, passage 5 hASCs were found to highly express VEGF165b (green) in culture, as seen with native retinal pericytes. VEGF165b is a splice variant of VEGF, that is an isoform of VEGF-A. DAPI counterstain identifies nuclei of individual cells (blue) b, retinal VEGF165b expression is known to diminish following removal from OIR. NOD SCID mice (n=4 per group) received intravitreal injections at postnatal day 5 (P5) with either hASCs or PBS carrier control, and then were exposed to OIR from P7 to P12. Pooled retinas (n=4 per group) were harvested from these mice at P17 and assayed for VEGF165b expression. hASC injected retinas show elevated levels of VEGF 165b as compared to retinas from their carrier control littermates, which may account in part for the observed accelerated recovery from damage due to OIR. Paracrine secretion of VEGF165b by hASCs may counter the well-characterized endogenous surge in vascular destabilizing VEGF165b following OIR.

Example 2, Supplementary FIG. 4: TGFβ treated mASCs stabilize preretinal neovascularization. a-d, Following mASC injection into C57Bl6 eyes at P17 (with contralateral PBS injections serving as carrier controls), neovascularization is quantified at P21 with the aid of the automated SWIFT_NV algorithm. e, PBS injection alone retains only 52.6% median neovascularization compared to mASC injected controls (p=0.016). f, DiI labeled mASCs incorporated into P21 retinal vasculature. (Scale bar=75 μm).

Example 3 Figures

Example 3, FIG. 1—Injected hASCs act as vascular support cells. Intraocularly injected hASCs (DiI, red) in OIR model retina survive at least 6-8 weeks (A) and migrate to and enwrap lectin staining vessels (B). Therapeutic hASCs (C, white arrow) express pericyte markers smooth muscle actin (C, SMA) and NG2 (D). Native pericytes seen without DiI (D, white arrows).

Example 3, FIG. 2—Lineage marked pericytes invade laser induced scar. Uninjured retinal vessels (A) show MyHCII/YFP lineage marking YFP pericytes (yellow) on vessels (lectin, blue). Following laser burn, the scar in whole mount shows prolific YFP signal (B, yellow). Superficial to the scar, retinal vessels exhibit diminished YFP Pericyte coverage (C) and presence of dual labeling lineage marker YFP and the macrophage and vessel marker lectin (blue). Expanded view of cell marked with white triangle (D, E) shows characteristic round macrophage nucleus.

Example 3, FIG. 3—A, B, C hASCs (green, middle panel A) and retinal pericytes (green, bottom panel A) pre-conditioned with TGF-B1 reduce proliferation of co-cultured endothelial cells (blue, arrested cells, pink, proliferating cells, panel A).

Example 3, FIG. 4—hASCs prevent hyperoxia induced retinal ablation. P2 OIR model retina treated with hASCs (B) versus control (A) show reduced avascular area (C).

Example 3, FIG. 5—mASCs decreased retinal capillary dropout. mASC (B) vs PBS injected diabetic retina show reduced capillary dropout at 8 weeks (C). In vivo imaging of PBS (D) vs mASC (E) injected retina show capillary preservation. Corresponding fields to D & E are shown dissected at higher resolution F & G.

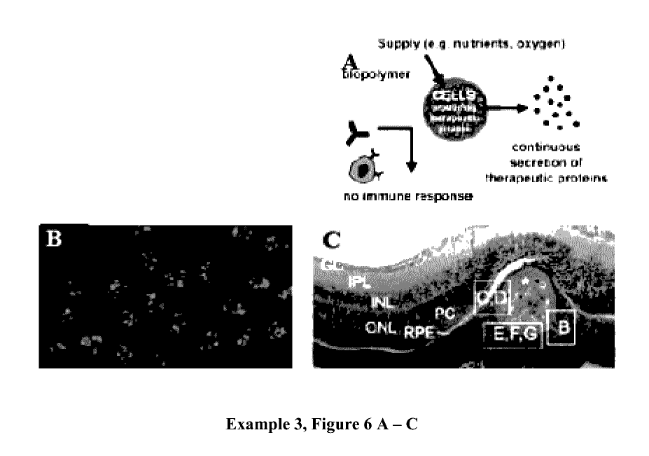

Example 3, FIG. 6—Encapsulated cells can be placed sub-retinally and exert paracrine regulatory effects. The construct (cartoon illustration, A) employs therapeutic cells placed in a biopolymer scaffold. Encapsulated cells can easily be followed through fluorescent markers (B). The construct can be injected posterior to the retina (C, *). Figures adapted from Fischer 2013.

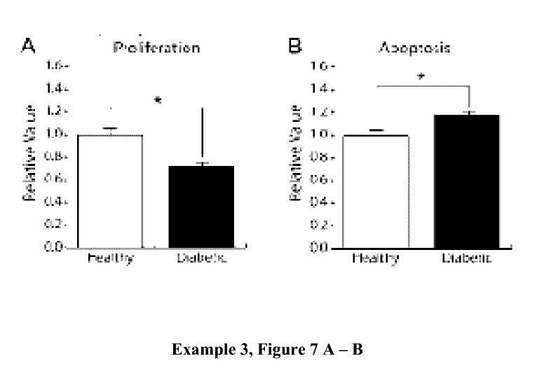

Example 3, FIG. 7—Healthy mASCs exhibit more proliferation and less apoptosis compared to diabetic mASCs. Relative number of cells in S phase determined by EdU incorporation assay (A). Cells undergoing apoptosis determined by TUNEL assay (B). mASCs from 3 healthy and 3 diabetic animals, values normalized to the healthy. *p<0.001, t-test.

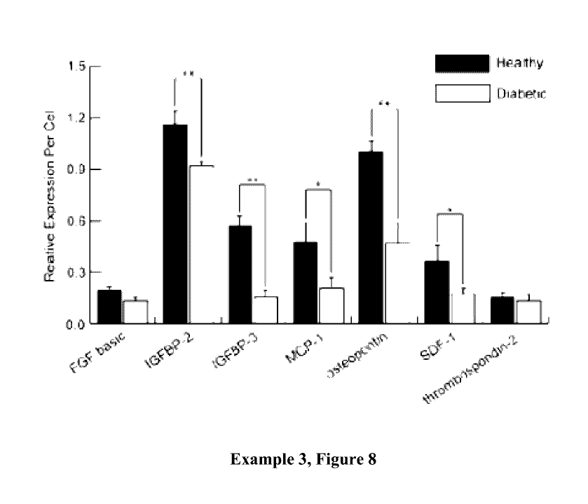

Example 3, FIG. 8—Angiogenic factor secretion decreased in diabetic mASCs. Expression of secreted angiogenic factor of healthy (WT mice) versus diabetic (Akimba mice) mASCs measured using high-throughput ELISA array. Samples consisted of 24 hour conditioned media. n=3, *p<0.05, **p<0.01, t-test.

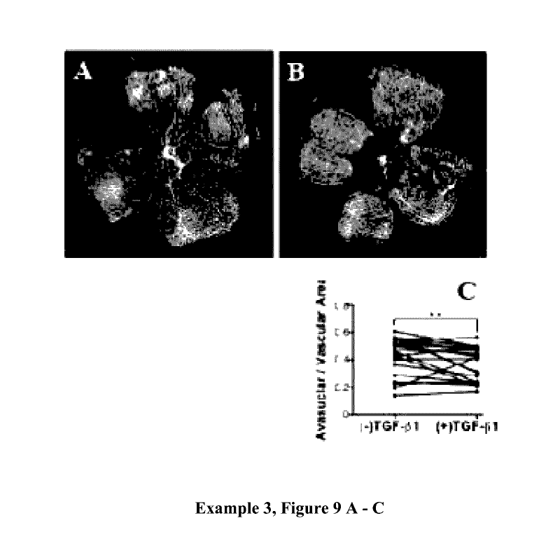

Example 3, FIG. 9—TGFβ preconditioning of ASCs enhances hyperoxic endothelial cell stabilization. P6 OIR model retina treated with ASCs conditioned without (A) and with (B) TGFβ. TGFβ conditioning enhances rescue of vascular area (C).

### DETAILED DESCRIPTION

### Abbreviations and Acronyms

AMD—age-related macular degeneration
ASC—adipose tissue-derived stem cell
ASCB—adipose stem/stromal cell blastema
ASC-MB—ASC-mesenchymal blastema or mesenchoid body
BMSC—bone marrow derived stem cell; also referred to as bone marrow
mesenchymal cells
CB—chimeric blastema
CNVM—choroidal neovascular membrane
DiI—1,1′-dioctadecyl-3,3,3′3′-tetramethylindocarbocyanine perchlorate
DMEM—Dulbecco's modified Eagle's medium
DR—diabetic retinopathy
ECM—extracellular matrix
ES—embryonic stem cell
FACS—fluorescent activated cell sorting
FBS—fetal bovine serum
FGF—fibroblast growth factor
gf—growth factor
hASC—human adipose-derived stem cell
HSC—hematopoietic stem cell
HS—human serum (also referred to as HmS herein)
HSA—human serum albumin
IGFBP-2—insulin-like growth factor binding protein 2
IGFBP-3—insulin-like growth factor binding protein 3
IL-1β—interleukin-1 beta
IL-6—interleukin-6
iPS—induced pluripotent stem cell
LHSC—human bone marrow-derived LIN—hematopoietic stem cell
mASC—murine ASC
MB—mesenchoid body
MCP-1—monocyte chemoattractant protein 1 (also referred to as CCL2)
OIR—oxygen induced retinopathy
PDGF—platelet-derived growth factor
PDGFRβ—PDGF receptor 0
PLA—processed lipoaspirate cells
PVR—proliferative vitreoretinopathy
RGS5—regulator of G-protein signaling 5
ROP—retinopathy of prematurity
RPE—retinal pigment epithelium
SCGF-β—stem cell growth factor-β
SDF—stromal cell-derived factor
SDF-1—also referred to as CXCL12
SFM—serum-free medium (also referred to as sf herein)
SMA—smooth muscle actin
SNiM—Self-organizing Niche Milieu, which is another term for ASC aggregates
SOM-B—Self-Organizing Mesenchymal Blastema (also referred to as “self-organizing mesenchoid bodies” and as SNiM herein)
SVF—stromal vascular fraction of human adipose tissue
TGFβ—transforming growth factor beta
TNFα—tumor necrosis factor alpha
ULA—ultra low attachment tissue culture plate
VEGF—Vascular endothelial growth factor

### DEFINITIONS

In describing and claiming the invention, the following terminology will be used in accordance with the definitions set forth below.

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated. Disease and disorders being treated by the additional therapeutically active agent include, for example, hypertension and diabetes. The additional compounds may also be used to treat symptoms associated with the injury, disease, or disorder, including, but not limited to, pain and inflammation.

“Adipose-derived stem cells”, also referred to as “adipose-derived stromal cells” herein, refer to cells that originate from adipose tissue. By “adipose” is meant any fat tissue. The adipose tissue may be brown or white adipose tissue, derived from subcutaneous, omental/visceral, mammary, gonadal, or other adipose tissue site. Preferably, the adipose is subcutaneous white adipose tissue. Such cells may comprise a primary cell culture or an immortalized cell line. The adipose tissue may be from any organism having fat tissue. Preferably, the adipose tissue is mammalian, more preferably, the adipose tissue is human. A convenient source of adipose tissue is from liposuction surgery, however, the source of adipose tissue or the method of isolation of adipose tissue is not critical to the invention.

As used herein, the term “adjuvant” refers to a substance that elicits an enhanced immune response when used in combination with a specific antigen.

As use herein, the terms “administration of” and or “administering” a compound should be understood to mean providing a compound of the invention or a prodrug of a compound of the invention to a subject in need of treatment.

The term “adult” as used herein, is meant to refer to any non-embryonic or non-juvenile subject. For example the term “adult adipose tissue stem cell,” refers to an adipose stem cell, other than that obtained from an embryo or juvenile subject.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom,” means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

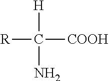

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

The term “autologous”, as used herein, refers to something that occurs naturally and normally in a certain type of tissue or in a specific structure of the body.

In transplantation, it refers to a graft in which the donor and recipient areas are in the same individual, or to blood that the donor has previously donated and then receives back, usually during surgery.

The term “basal medium”, as used herein, refers to a minimum essential type of medium, such as Dulbecco's Modified Eagle's Medium, Ham's F12, Eagle's Medium, RPMI, AR8, etc., to which other ingredients may be added. The term does not exclude media which have been prepared or are intended for specific uses, but which upon modification can be used for other cell types, etc.

The term “blastema”, as used herein, encompasses inter alia, the primordial cellular mass from which an organ, tissue or part is formed as well as a cluster of cells competent to initiate and/or facilitate the regeneration of a damaged or ablated structure.

The term “biocompatible,” as used herein, refers to a material that does not elicit a substantial detrimental response in the host.

The term “biodegradable,” as used herein, means capable of being biologically decomposed. A biodegradable material differs from a non-biodegradable material in that a biodegradable material can be biologically decomposed into units which may be either removed from the biological system and/or chemically incorporated into the biological system.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample,” as used herein, refers to samples obtained from a subject, including, but not limited to, skin, hair, tissue, blood, plasma, cells, sweat and urine.

The term “bioresorbable,” as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate may, for example, be redeposited as bone mineral, or by being otherwise re-utilized within the body, or excreted. “Strongly bioresorbable,” as the term is used herein, means that at least 80% of the total mass of material implanted is resorbed within one year.

The terms “cell” and “cell line,” as used herein, may be used interchangeably. All of these terms also include their progeny, which are any and all subsequent generations. It is understood that all progeny may not be identical due to deliberate or inadvertent mutations.

The terms “cell culture” and “culture,” as used herein, refer to the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture,” “organ culture,” “organ system culture” or “organotypic culture” may occasionally be used interchangeably with the term “cell culture.”

The phrases “cell culture medium,” “culture medium” (plural “media” in each case) and “medium formulation” refer to a nutritive solution for cultivating cells and may be used interchangeably.

A “compound,” as used herein, refers to any type of substance or agent that is commonly considered a drug, or a candidate for use as a drug, combinations, and mixtures of the above, as well as polypeptides and antibodies of the invention.

A “conditioned medium” is one prepared by culturing a first population of cells or tissue in a medium, and then harvesting the medium. The conditioned medium (along with anything secreted into the medium by the cells) may then be used to support the growth or differentiation of a second population of cells.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

The term “delivery vehicle” refers to any kind of device or material which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that may be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect. A “therapeutically effective amount” means an effective amount of an agent being used in treating or preventing a disease or disorder.

As used herein, an “essentially pure” preparation of a particular protein or peptide is a preparation wherein at least about 95%, and preferably at least about 99%, by weight, of the protein or peptide in the preparation is the particular protein or peptide.

The term “feeder cells” as used herein refers to cells of one type that are co-cultured with cells of a second type, to provide an environment in which the cells of the second type can be maintained, and perhaps proliferate. The feeder cells can be from a different species than the cells they are supporting. Feeder cells can be non-lethally irradiated or treated to prevent their proliferation prior to being co-cultured to ensure to that they do not proliferate and mingle with the cells which they are feeding. The terms, “feeder cells”, “feeders,” and “feeder layers” are used interchangeably herein.

As used herein, a “functional” molecule is a molecule in a form in which it exhibits a property or activity by which it is characterized.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

“Graft” refers to any free (unattached) cell, tissue, or organ for transplantation.

“Allograft” or “allogeneic” refers to a transplanted cell, tissue, or organ derived from a different animal of the same species.

“Xenograft” or “xenogeneic” refers to a transplanted cell, tissue, or organ derived from an animal of a different species.

The term “growth factor” as used herein means a bioactive molecule that promotes the proliferation of a cell or tissue. Growth factors useful in the present invention include, but are not limited to, transforming growth factor-alpha (TGF-α), transforming growth factor-beta (TGF-β), platelet-derived growth factors including the AA, AB and BB isoforms (PDGF), fibroblast growth factors (FGF), including FGF acidic isoforms 1 and 2, FGF basic form 2, and FGF 4, 8, 9 and 10, nerve growth factors (NGF) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor, stem cell factor (SCF), keratinocyte growth factor (KGF), skeletal growth factor, bone matrix derived growth factors, and bone derived growth factors and mixtures thereof. Some growth factors may also promote differentiation of a cell or tissue. TGF, for example, may promote growth and/or differentiation of a cell or tissue. Note that many factors are pleiotropic in their activity and the activity can vary depending on things such as the cell type being contacted, the state of proliferation or differentiation of the cell, etc.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCCS' and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “improved blood flow,” as used herein, refers to increased blood flow in a subject being treated according to the methods of the invention compared with the flow in a subject with an otherwise identical injury or condition not being treated according to the methods of the invention. Improved flow is determined by methods such as those described herein and can include less stasis, less sludging, or a combination of both, in the subject being treated compared with the untreated subject.

The term “ingredient” refers to any compound, whether of chemical or biological origin, that can be used in cell culture media to maintain or promote the proliferation, survival, or differentiation of cells. The terms “component,” “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

The term “inhibit,” as used herein, means to suppress or block an activity or function such that it is lower relative to a control value. The inhibition can be via direct or indirect mechanisms. In one aspect, the activity is suppressed or blocked by at least 10% compared to a control value, more preferably by at least 25%, and even more preferably by at least 50%.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

The term “inhibit a protein,” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a compound or cells of the invention by any number of routes and means including, but not limited to, intravitreal, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

The term “injury” refers to any physical damage to the body caused by violence, accident, trauma, or fracture, etc., as well as damage by surgery.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

Used interchangeably herein are the terms: 1) “isolate” and “select”; and 2) “detect” and “identify”.

The term “isolated,” when used in reference to cells, refers to a single cell of interest, or population of cells of interest, at least partially isolated from other cell types or other cellular material with which it naturally occurs in the tissue of origin (e.g., adipose tissue). A sample of stem cells is “substantially pure” when it is at least 60%, or at least 75%, or at least 90%, and, in certain cases, at least 99% free of cells other than cells of interest. Purity can be measured by any appropriate method, for example, by fluorescence-activated cell sorting (FACS), or other assays which distinguish cell types.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow and Lane, 1988, Antibodies, A Laboratory Manual, Cold Spring Harbor Publications, New York, for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

The term “low adherence, ultra low adherence, or non-adherence surface for cell attachment” refers to the ability of a surface to support attachment of cells. The term “non-adherence surface for cell attachment” means that the surface supports little if any cell attachment.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The terms “multicellular aggregate”, “multicellular sphere”, “blastema”, and “multicellular structure” are used interchangeably herein.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques. The term “progeny” of a stem cell as used herein refers to a cell which is derived from a stem cell and may still have all of the differentiation abilities of the parental stem cell, i.e., multipotency, or one that may no longer be multipotent, but is now committed to being able to differentiate into only one cell type, i.e., a committed cell type. The term may also refer to a differentiated cell.

The term “peptide” typically refers to short polypeptides.

The term “per application” as used herein refers to administration of cells, a drug, or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

The term “progeny” of a stem cell as used herein refers to a cell which is derived from a stem cell and may still have all of the differentiation abilities of the parental stem cell, i.e., multipotency, or one that may no longer be multipotent, but is now committed to being able to differentiate into only one cell type, i.e., a committed cell type. The term may also refer to a differentiated cell.

The term “propagate” means to reproduce or to generate.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

The term “regulate” refers to either stimulating or inhibiting a function or activity of interest.

A “reversibly implantable” device is one which may be inserted (e.g. surgically or by insertion into a natural orifice of the animal) into the body of an animal and thereafter removed without great harm to the health of the animal.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

As used herein, the term “solid support” when used in reference to a substrate forming a linkage with a compound, relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “solid support suitable for maintaining cells in a tissue culture environment” is meant any surface such as a tissue culture dish or plate, or even a cover, where medium containing cells can be added, and that support can be placed into a suitable environment such as a tissue culture incubator for maintaining or growing the cells. This should of course be a solid support that is either sterile or capable of being sterilized. The support does not need to be one suitable for cell attachment.

The term “solid support is a low adherence, ultralow adherence, or non-adherence support for cell culture purposes” refers to a vehicle such as a bacteriological plate or a tissue culture dish or plate which has not been treated or prepared to enhance the ability of mammalian cells to adhere to the surface. It could include, for example, a dish where a layer of agar has been added to prevent cells from attaching. It is known to those of ordinary skill in the art that bacteriological plates are not treated to enhance attachment of mammalian cells because bacteriological plates are generally used with agar, where bacteria are suspended in the agar and grow in the agar.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, a standard can be a known standard agent or compound which is administered or added to a control sample and used for comparing results when measuring said compound in a test sample. Standard can also refer to an “internal standard,” such as an agent or compound which is added at known amounts to a sample and is useful in determining such things as purification or recovery rates when a sample is processed or subjected to purification or extraction procedures before a marker of interest is measured.

The term “stimulate” as used herein, means to induce or increase an activity or function level such that it is higher relative to a control value. The stimulation can be via direct or indirect mechanisms. In one aspect, the activity or function is stimulated by at least 10% compared to a control value, more preferably by at least 25%, and even more preferably by at least 50%. The term “stimulator” as used herein, refers to any composition, compound or agent, the application of which results in the stimulation of a process or function of interest, including, but not limited to, wound healing, angiogenesis, bone healing, osteoblast production and function, and osteoclast production, differentiation, and activity.

A “subject” of diagnosis or treatment is an animal, including a human. It also includes pets and livestock.

As used herein, a “subject in need thereof” is a patient, animal, mammal, or human, who will benefit from the method of this invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “substituent” as used in the phrase “other cells which are not substituents of the at least one self-organizing blastema” refers to sub stituent cells of the blastema. Therefore, a cell which is not a substituent of a self-organizing blastema can be a cell that is adjacent to the blastema and need not be a cell derived from a self-organizing blastema.

A “surface active agent” or “surfactant” is a substance that has the ability to reduce the surface tension of materials and enable penetration into and through materials.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The use of the phrase “tissue culture dish or plate” refers to any type of vessel which can be used to plate cells for growth or differentiation.

The term “thermal injury” is used interchangeably with “thermal burn” herein.

“Tissue” means (1) a group of similar cells united to perform a specific function; (2) a part of an organism consisting of an aggregate of cells having a similar structure and function; or (3) a grouping of cells that are similarly characterized by their structure and function, such as muscle or nerve tissue.

The term “topical application,” as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous.”

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

As used herein, the term “wound” relates to a physical tear, break, or rupture to a tissue or cell layer. A wound may occur by any physical insult, including a surgical procedure or as a result of a disease, disorder condition.

Methods useful for the practice of the invention which are not described herein are also known in the art. Useful methods include those described in WO 2007/030652 (PCT/US2006/034915), WO 2007/019107 (PCT/US2006/029686), WO 2007/089798 (PCT/US2007/002572), and WO 2008/060374 (PCT US2007/021432), the methods of which are hereby incorporated by reference.

### EMBODIMENTS

Some retinal diseases, disorders, and injuries include, for example, diabetic retinopathy, retinopathy, arteriosclerotic retinopathy, hypertensive retinopathy, proliferative vitreoretinopathy, retinal tears, retinal detachment, macular degeneration, age related macular degeneration, inflammatory retinal disease, retinal vasculitis, retinal fibrosis, diffuse unilateral subacute neuroretinitis, cytomegalovirus retinitis, Stargardts, Best's Disease, Usher Syndrome, papilloedema, injury, surgical/treatment side effect, vitelliform maculopathy, retinitis pigmentosa, cone-rod dystrophy, retinal separation, retinal hypoxia, aberrant neovascularization of the retina, retinal scar formation, and retinoblastoma. In one aspect, the retinal disease, disorder, or injury prevented or treated in is diabetic retinopathy.

Debilitating retinopathies can involve progressive cellular degeneration leading to vision loss and blindness. These include, for example, diabetic retinopathy and choroidal neovascular membrane (CNVM). In one aspect, diabetic retinopathy may be classified as: 1) non-proliferative or background retinopathy, characterized by increased capillary permeability, edema, hemorrhage, microaneurysms, and exudates; or 2) proliferative retinopathy, characterized by neovascularization extending from the retina to the vitreous, scarring, fibrous tissue formation, and potential for retinal detachment.

In CNVM, abnormal blood vessels stemming from the choroid grow up through the retinal layers. The fragile new vessels break easily, causing blood and fluid to pool within the layers of the retina.

In another embodiment, the methods of the present invention may be used to prevent or treat macular degeneration. In one embodiment, macular degeneration is characterized by damage to or breakdown of the macula, which in one embodiment, is a small area at the back of the eye. In one embodiment, macular degeneration causes a progressive loss of central sight, but not complete blindness. In one embodiment, macular degeneration is of the dry type, while in another embodiment, it is of the wet type. In one embodiment, the dry type is characterized by the thinning and loss of function of the macula tissue. In one embodiment, the wet type is characterized by the growth of abnormal blood vessels behind the macula. In one embodiment, the abnormal blood vessels hemorrhage or leak, resulting in the formation of scar tissue if untreated. In some embodiments, the dry type of macular degeneration can turn into the wet type. In one embodiment, macular degeneration is age-related, which in one embodiment is caused by an ingrowth of choroidal capillaries through defects in Bruch's membrane with proliferation of fibrovascular tissue beneath the retinal pigment epithelium.

In another embodiment, the methods of the present invention may be used to prevent or treat retinopathy. In one embodiment, retinopathy refers to a disease of the retina, which in one embodiment is characterized by inflammation and in another embodiment, is due to blood vessel damage inside the eye. In one embodiment, retinopathy is diabetic retinopathy which, in one embodiment, is a complication of diabetes that is caused by changes in the blood vessels of the retina. In one embodiment, blood vessels in the retina leak blood and/or grow fragile, brush-like branches and scar tissue, which in one embodiment, blurs or distorts the images that the retina sends to the brain. In another embodiment, retinopathy is proliferative retinopathy, which in one embodiment, is characterized by the growth of new, abnormal blood vessels on the surface of the retina (neovascularization). In one embodiment, neovascularization around the pupil increases pressure within the eye, which in one embodiment, leads to glaucoma. In another embodiment, neovascularization leads to new blood vessels with weaker walls that break and bleed, or cause scar tissue to grow, which in one embodiment, pulls the retina away from the back of the eye (retinal detachment). In one embodiment, the pathogenesis of retinopathy is related to non-enzymatic glycation, glycoxidation, accumulation of advanced glycation end-products, free radical-mediated protein damage, up-regulation of matrix metalloproteinases, elaboration of growth factors, secretion of adhesion molecules in the vascular endothelium, or a combination thereof.

In one embodiment, retinopathy leads to macular edema, which in one embodiment, is swelling of the retina. In one embodiment, macular edema is characterized by retinal blood vessels that develop tiny leaks, which in one embodiment, allow blood and fluid to seep from the retinal blood vessels, and fatty material (called exudate) to deposit in the retina. In one embodiment, symptoms of macular edema comprise impaired or blurred vision.

In another embodiment, retinopathy refers to retinopathy of prematurity (ROP), which in one embodiment, occurs in premature babies when abnormal blood vessels and scar tissue grow over the retina. In one embodiment, retinopathy of prematurity is caused by a therapy necessary to promote the survival of a premature infant.

In another embodiment, retinopathy refers to arteriosclerotic retinopathy, which in one embodiment, is due to arteriosclerosis (hardening of the arteries). In another embodiment, retinopathy refers to hypertensive retinopathy, which in one embodiment, is due to high blood pressure. In another embodiment, retinopathy refers to solar retinopathy, while in another embodiment it refers to drug-related retinopathy.

In another embodiment, the methods of the present invention may be used to prevent or treat retinal detachment, including, inter alia, rhegmatogenous, tractional, or exudative retinal detachment, which in one embodiment, is the separation of the retina from its supporting layers. In one embodiment, retinal detachment is associated with a tear or hole in the retina through which the internal fluids of the eye may leak. In one embodiment, retinal detachment is caused by trauma, the aging process, severe diabetes, an inflammatory disorder, neovascularization, or retinopathy of prematurity, while in another embodiment, it occurs spontaneously. In one embodiment, bleeding from small retinal blood vessels may cloud the vitreous during a detachment, which in one embodiment, may cause blurred and distorted images. In one embodiment, a retinal detachment can cause severe vision loss, including blindness.

The development of diabetic retinopathy, which is characterized by vascular changes of the retinal capillary bed, is directly linked to the severity of hyperglycemia. The development of retinal capillary changes (microangiopathies) includes the appearance of the following histopathological and clinical lesions: selective pericyte loss, capillary basement membrane thickening, dilations/endothelial hypertrophy, permeability/hard exudates, capillary nonperfusion and occlusion/acellularity, microaneurysms/intraretinal hemorrhages, intraretinal microvascular abnormalities, ORMA shunts/dilated meshwork, cotton wool spots/ischemia, vessel-glial proliferation, extra retinal hemorrhages, glial-vitreal contraction, and macular edema. While many of these lesions are present in a number of ocular diseases, all of above-described lesions are only present together in DM.

Diabetic retinopathy includes the selective death of retinal capillary pericytes (mural cells or intramural pericytes). Exposure of pericytes to excess glucose or galactose results in apoptosis. The development and progression of diabetic retinopathy in both human patients and in dogs requires years to develop. This development is accelerated in galactosemic animals. For example, while retinopathy in diabetic dogs rarely progresses past the mild to moderate non-proliferative stage, retinal changes in galactose-fed dogs progress to the proliferative stage in essentially the same time period. The development and progression of diabetic retinopathy can be directly linked to hyperglycemic control.

The present application discloses the efficacy of pretreating populations of cells and discloses that cells from normal individuals can enhance healing and revascularization better than from a diseased individual. However, in one aspect, cells from subjects from with a disease or disorder can be used when the cells are subject to selection procedures or are treated with an agent to improve its ability to enhance revascularization. The present application further discloses agents useful for such purposes.

In one aspect, the cells administered to a subject are allogeneic. In one aspect, the cells are autologous.

In one aspect, the use of allogeneic cells can be optimized by lowering the infused dose of cells.

In one embodiment, the engraftment comprises an allogeneic donor engraftment. In another, it is autologous.

In one embodiment, at least two different populations of cells are administered to a subject. For example, the two populations can be different donors or from different isolates from the subject.

In one aspect, when two populations are used, they are at least partially HLA matched.

The number of cells administered can be varied depending on, for example, the source of the cells, the age of the subject, and the health of the subject. The number of cells can be based on the age, sex, health, weight, or other parameters, or can be based on a unit dose. The amount of time that a population of cells is exposed to an agent, such as TGF-β, can vary, depending on the particular circumstances for the engraftment. For example, exposure can be from several minutes to several hours. In one aspect, cells are exposed to the agent for about 30 minutes. In one aspect, the cells were frozen and then thawed before being contacted with the agent. In one aspect, the agent is also administered with the cells or is administered before or after the cells are administered.

The ASCs can be administered to subjects in amounts (or numbers) effective to treat the patient, as described herein. The numbers of cells necessary for treatment will depend on a number of factors including the severity of the symptoms experienced by the subject, the degree of enrichment of the desired cell type in the administered population, the age and/or weight of the patient, and the like. In one embodiment, cell numbers in the range of about 1 to 10×10⁶ or about 2 to 8×10⁶ can be administered to the subject. Thus depending on the particular patient and the population being administered, the number of cells administered can be about 2×10⁶, about 3×10⁶, about 4×10⁶, about 5×10⁶, about 6×10⁶, about 7×10⁶, about 8×10⁶, about 9×10⁶, about 10×10⁶, about 20×10⁶, about 50×10⁶, or about 100×10⁶.

In another embodiment, the number of cells can be injected base on weight (kg) of the subject. In one embodiment, cell numbers in the range of about 1×10⁴ to 10×10⁶/kg or about 2×10⁴ to 8×10⁶/kg can be administered to the subject. Thus depending on the particular patient and the population being administered, the number of cells administered can be about 1×10⁴/kg, 2×10⁴/kg, 3×10⁴/kg, 4×10⁴/kg, 5×10⁴/kg, 6×10⁴/kg, 7×10⁴/kg, 8×10⁴/kg, 9×10⁴/kg, 1×10⁵/kg, 2×10⁵/kg, 3×10⁵/kg, 4×10⁵/kg, 5×10⁵/kg, 6×10⁵/kg, 7×10⁵/kg, 8×10⁵/kg, 9×10⁵/kg, 1×10⁶/kg, 2×10⁶/kg, 3×10⁶/kg, 4×10⁶/kg, 5×10⁶/kg, 6×10⁶/kg, 7×10⁶/kg, 8×10⁶/kg, 9×10⁶/kg, 1×10⁷/kg, or about 5×10⁷/kg.

In one embodiment, a unit dose of cells is administered that is not dependent on weight, amount of retinal vasculature damage, etc.

In one embodiment, a subject is subjected to a conditioning regimen prior to administration of a population of cells that has been contacted with at least one agent. In one embodiment, more than one population of cells can be administered, and as described herein one or more of the populations can be contacted with an agent prior to administration to the subject.

In one embodiment, an effective amount of at least one growth factor, cytokine, hormone, or extracellular matrix compound or protein is administered as part of the composition or is used to pretreat cells. In one aspect, a combination of these agents is used or they are included in the mixture of the invention.

As disclosed herein, cells can be pretreated with TGFβ, prior to being administered to a subject. The amount of time they are pretreated and the concentration of TGFβ used can be varied. For example, cells could be pretreated for about 1 hour up to about 96 hours, about 2 hours to about 72 hours, about 3 hours to about 48 hours, or about 4 to about 24 hours, or about 5 hours to about 12 hours. The amount of TGFβ can be varied from about 0.1 ng/ml to about 50 ng/ml, about 0.2 ng/ml to about 25 ng/ml, about 0.3 ng/ml to about 20 ng/ml, about 0.4 ng/ml to about 15 ng/ml, about 0.5 ng/ml to about 10 ng/ml, about 0.6 ng/ml to about 9 ng/ml, about 0.7 ng/ml to about 8 ng/ml, about 0.8 ng/ml to about 5 ng/ml, and about 0.9 mg/ml to about 1.0 ng/ml.

The present application further provides for the use of conditioned medium from adipose derived stem cells to prevent or treat diseases, disorders, and injuries of the retina or associated with the retina. Additionally, the present application provides for the use of combination therapies, including administering both adipose derived stem cells and adipose-derived stem cell conditioned medium to the subject. One of ordinary skill in the art will appreciate that the administration can be simultaneous or can occur at different times.

As disclosed herein, adipose-derived stem cell-conditioned medium comprises multiple factors for use in preventing or treating diseases, disorders, and injuries of the retina, such as IGFBP-2, IGFBP-3, MCP-1, osteopontin, and SDF-1. In addition to administering the conditioned medium, any one or more of these factors can be used to supplement the conditioned medium and other growth factors and cytokines can be administered as well. Additional growth factors and cytokines include, but are not limited to, VEGF, PDGF, FGF, TNFα, IL-6, and endothelin-1.

The present invention encompasses the use of the proteins described herein as well as biologically active homologs and fragments thereof. The proteins are known in the art. NCBI GenBank Accession numbers for the human proteins described herein, including some precursors, fragments, and isoforms are provided below.

TGFβ—Accession: AAA36738.1, 431 aa.

Insulin-like growth factor-binding protein 2 (IGFBP-2)—Accession: NP_000588.2, 328 aa.

IGFBP-3—The encoded protein includes a 27-residue signal peptide followed by the 264-residue mature protein. IGFBP-3 shares with the other five high-affinity IGFBPs and a 3-domain structure:
isoform a precursor, 297 aa protein, Accession: NP_001013416.1
isoform b precursor, 291 aa protein, Accession: NP_000589.2 and Accession: P17936.2

Monocyte Chemoattractant Protein-1 (also referred to as CCL2). Mature human MCP-1 is composed of 76 amino acids and is 13 kDa in size. The precursor also has a 23 amino acid signal peptide; Accession: AAB20651.1, 99 aa

Osteopontin—

300 aa protein, Accession: AAA59974.1 or AAC28619.1 or NP_000573.1

314 aa protein, Accession: AAA86886.1 or BAA03554.1 or P10451.1 or NP_001035147.1

273 aa protein, Accession: BAH58215.1 or BAE45628.1 or NP_001238758.1

287 aa protein, Accession: NP_001035149.1

isoform 5, 327 aa protein, Accession: NP_001238759.1

SDF-1—Processed forms SDF-1-beta (3-72) and SDF-1-alpha (3-67) are produced after secretion by proteolytic cleavage of isoforms Beta and Alpha, respectively.

93 aa protein, Accession: P48061.1

VEGF—There are multiple isoforms of VEGFA that result from alternative splicing of mRNA from a single, 8-exon VEGFA gene. These are classified into two groups which are referred to according to their terminal exon (exon 8) splice site: the proximal splice site (denoted VEGFxxx) or distal splice site (VEGFxxxb). In addition, alternate splicing of exon 6 and 7 alters their heparin-binding affinity and amino acid number (in humans: VEGF121, VEGF121b, VEGF145, VEGF165, VEGF165b, VEGF 189, VEGF206). VEGFA has been classically described as having four main splice variants (121, 165, 189, and 206), although other splice variants have been described as present. VEGF165b, a form of VEGF165, is a splice variant on which exon 8 has a 6-amino acid difference from the typical VEGF165. VEGF165b is an antiangiogenic VEGFA isoform.

splice variant VEGF 117, 143 aa protein, Accession: AAP86646.1

precursor, 232 aa protein, Accession: P15692.2

isoform s, 163 aa protein, Accession: NP_001273973.1

isoform g, 371 aa protein, Accession: NP_001028928.1

isoform e, 354 aa protein, Accession: NP_001020540.2

isoform d, 371 aa protein, Accession: NP_001020539.2

isoform c, 389 aa protein, Accession: NP_001020538.2

isoform b, 395 aa protein, Accession: NP_003367.4

isoform a, 412 aa protein, Accession: NP_001020537.2

isoform o precursor, 191 aa protein, Accession: NP_001165100.1

isoform m precursor, 174 aa protein, Accession: NP_001165098.1

isoform l precursor, 191 aa protein, Accession: NP_001165097.1

isoform k precursor, 209 aa protein, Accession: NP_001165096.1

isoform j precursor, 215 aa protein, Accession: NP_001165095.1

isoform i precursor, 232 aa protein, Accession: NP_001165094.1

isoform h, 317 aa protein, Accession: NP_001165093.1

isoform f, 327 aa protein, Accession: NP_001020541.2

isoform p precursor, 137 aa protein, Accession: NP_001165101.1

isoform n precursor, 147 aa protein, Accession: NP_001165099.1

PDGF is a dimeric glycoprotein composed of two A (-AA) or two B (-BB) chains or a combination of the two (-AB).

PDGF-A

isoform 2 preproprotein, 196 aa protein, Accession: NP_148983.1

isoform 1 preproprotein, 211 aa protein, Accession: NP_002598.4

234 aa protein, Accession: AAI09247.1

PDGFB

241 aa protein, Accession: 1109245A

isoform 2 preproprotein, 226 aa protein, Accession: NP_148937.1

FGF—The FGFs are heparin-binding proteins and interactions with cell-surface-associated heparan sulfate proteoglycans have been shown to be essential for FGF signal transduction. FGF1 is also known as acidic, and FGF2 is also known as basic fibroblast growth factor. One important function of FGF1 and FGF2 is the promotion of endothelial cell proliferation and the physical organization of endothelial cells into tube-like structures. They thus promote angiogenesis, the growth of new blood vessels from the pre-existing vasculature. FGF 1 and FGF2 are more potent angiogenic factors than vascular endothelial growth factor (VEGF) or platelet-derived growth factor (PDGF). When the term FGF is used herein, it encompasses the use of FGF1 or FGF2 as well as biologically active homologs and fragments thereof.

FGF1 isoform 1 precursor, 155 aa protein, Accession: NP_001244139.1 or AAH32697.1 or P05230.1

FGF2, 288 aa protein, Accession: NP_001997.5

TNFα—233 aa protein, Accession: NP_000585.2 or AAA61200.1.

Interleukin-6

precursor, 212 aa protein, Accession: NP_000591.1 or AAD13886.1.

211 aa protein, Accession: AFF18412.1

185 aa protein, Accession: AAB30962.1

Endothelin-1

212 aa protein, Accession: P05305.1 or AAA52339.1

partial, 51 aa protein, Accession: AAA52341.1.

The volume of conditioned medium to be used can be varied based on a number of factors including the age, health, sex, and weight of the subject, the particular disease, disorder or condition being treated, and on the amount of factors secreted into the medium. Tests are described herein to determine the type and amount of each factor of interest secreted into the medium and other tests are known in the art as well. Additionally, steps can be taken to increase the amount or concentration of factors present, for example, by increasing the time that the medium is conditioned, increasing the number of cells being used, or by concentrating the medium and factors once the medium is conditioned. One of ordinary skill in the art can determine the volume of liquid to be administered based on the parameters described above. Additionally, the conditioned medium and factors therein can be at least partially purified. The invention further encompasses adding conditioned medium or the partially purified factors directly into a pharmaceutical composition being administered to the subject, including one that comprises adipose-derived stem cells and/or additional therapeutic agents. The volume of condition medium to be administered can vary from, for example, about 0.01 ml to about 1.0 ml, about 0.02 ml to about 0.9 ml, about 0.03 ml to about 0.8 ml, about 0.04 to about 0.7 ml, about 0.05 ml to about 0.6 ml, about 0.06 ml to about 0.5 ml, about 0.07 ml to about 0.4 ml, about 0.08 to about 0.3 ml, about 0.09 ml to about 0.1 ml.

In one embodiment, a population of cells comprising ASCs is contacted with an effective amount of at least two agents prior to administration to a subject. In one embodiment, two or more populations of cells are each contacted with at least one incorporation enhancing agent prior to administration of the cells to the subject. In one embodiment, when two or more populations of cells are used, at least one of the populations of cells is contacted with at least two incorporation enhancing agents. In one embodiment, when two or more populations of cells are administered to a subject, at least one of the populations of cells is not contacted with an incorporation enhancing agent. In one aspect, instead of using a cell incorporation enhancing agent, one or more revascularization enhancing agents are administered with the cells or after the cells are administered.

In one embodiment, the method of the invention enhances the rate of retinal revascularization in a subject.

In one embodiment, a subject is being treated for a disease, condition, or disorder including, but not limited to, those where there is a decrease in retinal vasculature or an injury to retinal vasculature.

In one embodiment, when cells are administered to a subject, at least one therapeutic agent is also administered.

In one embodiment, prior to administration a population of cells is enriched, for example, to enrich it for pericytes. In one embodiment, an enriched population of cells is contacted with an agent prior to administration wherein said agent enhances incorporation into the retina or an agent that increase the pericyte phenotype of the ASCs. In another embodiment, an enriched population of cells is not contacted with an agent prior to administration to a subject.

One of ordinary skill in the art will appreciate that enriched populations of cells can be used as described herein for other populations of cells, including the descriptions for using one of more populations, contacting one or more populations with an engraftment enhancing agent at different concentrations, etc., when the populations have not been subjected to an enrichment procedure.

The present invention further provides kits. In one embodiment, the invention provides kits for enhancing ASC incorporation into the retina. In one aspect, the kit comprises at least one population of cells comprising ASCs. The kit further provides at least one agent effective for stimulating or enhancing incorporation. In one embodiment, the kit provides at least one factor, such as a growth factor or chemokine, that enhances retinal revascularization. The kit may also include conditioned medium from ASCs. In one aspect, the conditioned medium may be purified and in another it may be concentrated. The kit optionally provides a pharmaceutically acceptable carrier, an applicator, and an instructional material for the use thereof.

The invention is also directed to methods of administering the compounds, cells, proteins and peptides (collectively referred to as compounds) of the invention to a subject.

The present invention encompasses, inter alia, treatment of retinopathies resulting from diabetes, preventative therapy for premature infants, regeneration of damaged retinas from ischemia/reperfusion injuries (retinal artery or vein occlusion), regeneration of ocular vascular tissue from trauma, treatment of retinal vascular damage due to retinal hypertension, treatment of age-related macular degeneration, treatment of anterior segment and cornea and sclera vasculopathies, and treatment of choroidal vasculopathies.

The cellular milieu from which ASCs, including hASCs, are derived are known to be particular to adipose tissue and comprise a substantially different population than bone marrow derived cells. Both mechanism of cellular and vascular repair and phenotypic identity of hASCs, while sharing some characteristics with bone marrow derived cells, is distinct. In an ischemic hindlimb model they appear to promote angiogenesis, similar to bone marrow derived stem cells, through secretion of angiogenic and anti-apoptotic factors including GM-CSF, HGF, bFGF, VEGF, and TGFβ.

Importantly, a number of in-vitro and in-vivo studies have shown that hASCs can assume a pericyte-like role in vascular repair. hASCs co cultured with human microvascular endothelial cells on matrigel co-assembled a microvascular network, enhanced stability of the microvascular network, showed a pericyte like localization to the abluminal side of cords, and expressed pericyte specific markers. Recently, hASCs have been shown to exhibit similar behaviors in vivo in nude rat mesenteries that were stimulated to undergo microvascular remodeling. Within the mesentery the hASCs show abluminal localization along blood vessels and pericyte-like wrapping of the vessels. They also express characteristic perivascular-cell markers in vivo including smooth muscle actin and NG2. Similar to pericytes, hASCs express PDGF receptor B, which accounts for their increased migration in response to PDGF-BB in vitro. This data taken together strongly suggests that hASCs function in vivo in a pericyte-like role along blood vessels and may be capable of stabilizing retinal vasculature in diseases such as retinopathy of prematurity and diabetic retinopathy.

In one embodiment, the present invention encompasses applying the regenerative effects of hASCs to control the pathological effects that occur in ocular disease. As pluripotent cells hASCs can potentially replace any retinal element that is damaged as part of the eye disease process which includes but is not limited to retinal pigment epithelium, bipolar cells, amacrine cells, horizontal cells, retinal ganglion cells, pericytes, endothelial cells, photoreceptor cells, glial support cells, astrocytes, endothelial and supporting vascular cells. In addition to direct repair of cellular damage, hASCs can alter the cellular environment to provide a supportive environment for repair of ocular pathology. This can occur through both direct cell-to-cell contact, autocrine, and paracrine mechanisms.

Retinal degeneration involves a loss of multiple retinal elements including, but not limited to retinal pigment epithelial cells, photoreceptors, and associated neural and vascular elements. In one embodiment, the present invention encompasses treating retinal degenerations; both acquired such as non-exudative macular degeneration and inherited such as retinitis pigmentosa and Stargardt's disease through direct replacement of damaged cellular elements with hASCs. We also propose that hASCs are capable of providing support to remaining cellular elements through modification of the retinal environment such as modifying the apoptosis cascade, secreting stimulatory or growth factors necessary for survival, or reestablishing retinal architectonics to allow for continued survival and repair of cellular damage.

Glaucoma and optic neuropathies involves a loss of retinal ganglion cells through a multifactorial process. hASCs can potentially directly replace damaged retinal ganglion cells through differentiation into this cell type. They can also stabilize retinal ganglion cells loss through differentiation into a supporting cell type or modification of the cellular environment through secretion of relevant factors necessary for continued survival of these cells including but not limited to anti-apoptotic agents and growth factors.

Ocular vasculopathies such as exudative macular degeneration, diabetic retinopathy, and retinopathy of prematurity are also potentially amenable to treatment with hASCs. Many vascular pathologies of the eye are characterized by immature, unstable vessel beds, which are continually growing and regressing in an attempt to supply nutrients to the relevant tissue. Through the incorporation of hASCs, and the signals provided therein by direct incorporation and indirect secretion of relevant factors, it is possible to stabilize the blood vessel network. The result is a decrease in vessel permeability and the number of structurally-compromised vessels per tissue area, as abnormal vessels naturally regress, leaving only the mature vessels supported by injected hASCs. The hASCs function to provide a perivascular cell population to replace absent or non-functional host perivascular cells as seen in disorders such as diabetic retinopathy, and in additional ocular disorders such as retinopathy of prematurity or age-related macular degeneration, the hASCs provide a source of competent endothelial and perivascular cells to reinforce the fragile and leaky blood vessels. In any ocular pathological application, secretions from the hASCs act to foster a mature angiogenic environment.

The advantage of using hASCs in these applied areas are that: 1) these cells are readily available from liposuction procedures, 2) with minimal invasiveness they can be harvested in large quantities compared to bone marrow-derived progenitor cells, 3) there exists the potential for autologous transplantation in the clinic, 4) this cell type is relatively abundant in most humans, and 5) hASCs are easily expanded in vivo.

The present invention discloses compositions and methods which encompass the preparation, delivery, and use of hASCs for treatment of ocular diseases and disorders. Restoration of ocular function can occur through a number of mechanisms of repair that have been demonstrated for these cells. This invention includes for illustrative purposes, but is not limited to, treatment of common ocular diseases including diabetic retinopathy, retinopathy of prematurity, macular degeneration, vascular occlusive disease, and hereditary retinal degenerations. The application is intended to cover all potential uses of hASCs for treatment of ocular disease with no limitation implied by description of a particular disease or a particular mechanism of repair. The present application covers all variants and combination of proposed mechanisms of treatment of disease for these cells for ocular disease. This invention views treatment as consisting of both structural replacement of damaged components of the eye (including but not limited to endothelial cells, retinal ganglion cells, perivascular support cells, glial cells, and neural cells), as well as establishment of a microenvironment conducive to ocular repair (including but not limited to secretory activities of the hASCs, architectonic support, and contact mediated changes on retinal cellular elements).

Adipose tissue (fat) embodies all of the requisite qualities of an ideal autologous cell source. With the prevalence of obesity reaching epidemic proportions in the US and globally, adipose tissue is abundant, expendable and replenishable. In addition, it is safe and easy to harvest through minimally invasive aspiration/suction techniques, a procedure actually perceived by many as appealing. Recent work shows that an average of 400,000 ASCs can be obtained from every milliliter of suction-harvested adipose tissue. Given the fact that up to two liters of adipose tissue can be safely removed from an individual with a single, minimally invasive outpatient procedure, nearly 800 million ASCs can be readily obtained with minimal morbidity. These unmatched qualities make ASCs practical and ideally suited for translational self-cell therapies.

Several independent research groups have now published on the characterization, self-renewal and multilineage developmental plasticity of stem (stromal) cells isolated from human subcutaneous adipose tissue. Their developmental spectrum includes lineages characterized as osteogenic, chondrogenic, myogenic, angiogenic, neurogenic and most pertinent to this proposal, adipogenic. In addition to their developmental plasticity, human ASCs are also known to produce a wide variety of soluble and insoluble factors that favorably impact the repair of damaged tissues through the modulation of angiogenesis, inflammation, apoptosis, cell homing, cell proliferation, and cell migration. For example, Rehman et al. have shown that hASCs secrete several angiogenic and anti-apoptotic proteins that can be up-regulated under hypoxic and other culture conditions. Angiogenesis is known to be tightly and critically linked to adipogenesis.

Adult human extramedullary adipose tissue-derived stromal cells represent a stromal stem cell source that can be harvested routinely with minimal risk or discomfort to the patient. Pathologic evidence suggests that adipose-derived stromal cells are capable of differentiation along multiple lineage pathways. Adipose tissue is readily accessible and abundant in many individuals.

Adipose tissue offers many practical advantages for tissue engineering applications. First, it is abundant. Second, it is accessible to harvest methods with minimal risk to the patient. Third, it is replenishable. While stromal cells represent less than 0.01% of the bone marrow's nucleated cell population, there are up to 8.6×10⁴ stromal cells per gram of adipose tissue. Ex vivo expansion over 2 to 4 weeks yields up to 500 million stromal cells from 0.5 kilograms of adipose tissue. These cells can be used immediately or cryopreserved for future autologous or allogeneic applications.

Adipose derived stromal cells also express a number of adhesion and surface proteins. These include, but are not limited to, cell surface markers such as CD9; CD29 (integrin beta 1); CD44 (hyaluronate receptor); CD49d,e (integrin alpha 4, 5); CD54 (ICAM1); CD55 (decay accelerating factor); CD105 (endoglin); CD106 (VCAM-1); CD166 (ALCAM) and HLA-ABC (Class I histocompatibility antigen); and cytokines such as interleukins 6, 7, 8, 11; macrophage-colony stimulating factor; GM-colony stimulating factor; granulocyte-colony stimulating factor; leukemia inhibitory factor; stem cell factor and bone morphogenetic protein. Many of these proteins have the potential to serve a hematopoietic supportive function and all of them are shared in common by bone marrow stromal cells.

The adipose tissue-derived stromal cells useful in the methods of invention can be isolated by a variety of methods known to those skilled in the art such as described in WO 00/53795. In a preferred method, adipose tissue is isolated from a mammalian subject, preferably a human subject. A preferred source of adipose tissue is omental adipose. In humans, the adipose is typically isolated by liposuction. If the cells of the invention are to be transplanted into a human subject, it is preferable that the adipose tissue be isolated from that same subject to provide for an autologous transplant. Alternatively, the transplanted cells are allogeneic.

The specific vehicle or method in which the cells are delivered may vary according to the individual application. This application encompasses modifications to vehicle structure and includes, but is not limited to, changes to the culture medium itself, addition of cellular support factors to this medium, and addition of support cells beyond the hASC to increase both efficacy of cellular survival, migration, integration, differentiation, and functionality.

The method of delivery of cells to the target tissue can be determined by one of ordinary skill in the art based on factors such as type of pathology, disease, disorder, or injury, age, sex, or general health of the subject, and may be accomplished, for example, through intravitreal injection, subretinal or subchoroidal implantation through transscleral delivery, intravenous delivery, retro-orbital implantation, intravitreal implantation in a sustained delivery device, and subdural implantation. Delivery may be applied as a single injection, repeated injections, or continuous delivery thru an intraocular or extraocular device. Sustained delivery may consist of time-released delivery of cells themselves, or chemical factors produced by the cells to the target area for treatment. The choice of a particular means of vehicle delivery, timing of delivery, and containment device for storage of cells either extra or intraocular is determined by efficacy as measured for the individual application. It is not specific to the individual application of hASCs to a particular ocular disease.

Maintenance of hASC function may require additional support elements consisting of chemical, cellular, or architectural changes to the implantation environment. This application includes all such modifications or future enhancements to maintaining the effects of hASC, either on the hASCs themselves or the ocular elements in their surrounding environment.

hASCs once at the target eye tissue will serve to reestablish function of the eye. Reestablishing function includes, but is not limited to, modification of the cellular microenvironment surrounding the hASC, repair of damaged cellular elements within the eye, replacement of damaged cellular elements within the eye either directly or indirectly, secretion of cellular support factors to support both the hASC and ocular elements, establishment of architectonics necessary for restoration of function, induction of additional factors or cellular migration to the site of pathology, stabilization of existing damaged or undamaged elements of the eye to allow continuation of ocular function.

Application of hASCs to a particular ocular disease is independent of the particular class of hASCs used, the means for selection of hASC class, delivery mechanism, vehicle for delivery, or factors required for maintenance of hASC effects. Examples of potential ocular diseases that we propose to use hASCs for regenerative repair include but are not limited to neural degeneration such as glaucoma and optic neuropathies such as Leber's optic neuropathy and optic neurites, vascular disease such as diabetic retinopathy, exudative macular degeneration, retinopathy of prematurity, and vascular occlusive disease, retinal degenerations such as retinitis pigmentosa, Stargardt's disease, hereditary retinal degenerations, nonexudative macular degeneration, congenital stationary night blindness. The regenerative repair mechanism may involve or more of the above mentioned mechanisms of repair including direct replacement of damaged cellular elements,

The following illustrative examples describe potential uses for hASCs in treating common ocular disease:

Glaucoma: Glaucoma involves degeneration of retinal ganglion cells due a multitude of environmental and genetic risk factors. The most salient risk factor, and the only one currently treatable, is intraocular pressure. Focus has been placed on both neuroprotection of existing retinal ganglion cells through growth factor or anti-apoptotic administration, as well as retinal ganglion cell regeneration. However, no current therapies are available to treat these disease targets. hASCs are pluripotent and can differentiate into multiple cell types. Through modification of culture conditions they can potentially differentiate into retinal ganglion cells. These cells can replace damaged or lost retinal ganglion cells to restore retinal function. Additionally, hASCs are known to secrete multiple anti-apoptotic and growth factors that are known to be important to retinal ganglion cell survival. hASCs functioning as support cells within the retina can create the cellular microenvironment through secretory, contact, and architectural mechanisms to allow restoration of function or enhanced functioning of damaged retinal ganglion cells. Additionally, modification of the cellular microenvironment may directly modify either on an architectural, cellular, or chemical level the ocular factors that result in damage to retinal ganglion cells. These ocular factors could include but are not limited to changes in ocular blood flow, regulation of control of intraocular pressure, recruitment of additional support cells to the retina to allow continued survival of retinal ganglion cells, modification of the apoptotic cascade, changes in vascular endothelial growth factor or other growth factor levels.

Diabetic retinopathy: hASCs have been shown to partially differentiate in conditions of ischemic tissue damage into a cell type phenotypically similar to a pericyte. This identity is made is by both morphologic criteria as well as pericyte marker identification on these cells. In diabetic retinopathy, there is preferential loss of pericyte support cells due to hyperglycemic damage. Perivascular cells are generally responsible for stabilizing and maintaining structural integrity of the blood vessels, so the ability of hASCs to differentiate into such cells and secrete relevant stabilizing factors makes them an attractive cellular therapy option for vascular eye pathologies. In addition, the ability of hASCs to contribute to the re-endothelialization of damaged and leaky blood vessels, a pathological condition found in many ocular disorders, would be therapeutically beneficial. Extravascular non-incorporating hASCs also create a supportive environment for vascular stabilization and repair through paracrine mechanisms which may be relevant to treatment of diabetic retinopathy.

Retinal neovascularization that occurs in diabetic retinopathy results in collections of aberrant and immature retinal vessels. These vessels lack adequate perivascular support cells and thus are subject to destabilization and changes in growth factor concentration levels. Progression of neovascularization leads to vitreous hemorrhage and fibrovascular scarring which causes loss of vision. Neovascularization is a programmed response to retinal ischemia, but lacks the needed vessel maturation to stabilize them into functional blood vessels. We propose that hASCs may in stabilize these vessels, allowing them to provide functional vascular support to the retina. This is proposed to occur through chemical and cellular modification of the microenvironment, and in particular by direct coverage of these immature vessels with hASCs differentiated into a perivascular support phenotype. Coverage of the neovascularization with perivascular support cells will decrease responsiveness and dependence on extravascular factors such as vascular endothelial growth factor, and decrease the permeability of these vessels which causes retinal edema and exudate. This represents a novel approach to therapies for ocular disease in that stabilization of preexisting immature neovascularization, rather than directed growth of new blood vessels is the goal.

Retinopathy of prematurity: Initially in ROP, hyperoxia leads to the degeneration of retinal blood vessels. Subsequently, avascular retinal tissue becomes ischemic, vascular endothelial growth factor (VEGF) levels increase, and abnormal vessels grow in response to increased VEGF. Hyperoxia has been shown to cause regression of blood vessels across multiple animal models of this process. Hyperoxia is known to decrease levels of VEGF in the retina, which causes the destabilization of newly formed vessels. This suggests that pericytes may be the key to protecting the immature blood vessels in ROP from the damage caused by changes in oxygen levels. As neonatal vascular development proceeds, there is an inherent delay between blood vessel formation and subsequent covering of these vessels with pericyte support cells. A number of studies have suggested that the extent of pericyte coverage of retinal vessels alters their susceptibility to both hypoxic and hyperoxic insults. These findings indicate that immature retinal vessels lacking pericyte coverage regress in response to the low VEGF levels created by hyperoxia. We propose in this invention to treat retinopathy of prematurity using hASCs. The mechanisms through which hASCs may treat ROP may be multiple including protection of preexisting immature vessels or halting development of aberrant neovascularization through modifications of the cellular microenvironment. In particular hASCs differentiating into pericyte like cells can potentially increase pericyte coverage on immature vessels to stabilize them. By stabilizing immature vessels both normal and aberrant neovascularization, ischemia can be either reversed at early or late stages of the disease. As with diabetic retinopathy, immature neovascularization can be stabilized into functional vessels to correct the ischemia of peripheral retinal tissue.

Macular degeneration and retinitis pigmentosa: In macular degeneration and retinitis pigmentosa retinal elements are gradually lost due to inflammatory causes as well as genetic defects within particular cells in the retina. Modification of the hASC culture condition may allow generation of multiple retinal elements particular to these diseases including but not limited to photoreceptors and retinal pigment epithelial cells. hASCs are proposed to integrate into the retina in areas where these retinal elements have been lost and also be capable of reestablishing functional connections within the retinal network to restore function. hASCs are also proposed to secrete multiple chemokines and create a supportive environment that may allow slowing or halting of further degeneration of these elements. hASCs may also enhance function of other elements within the retina allowing partial or complete compensation for abnormal retinal function.

The cells of the present invention may be administered to a subject alone or in admixture with a composition useful in the repair of wounds and other defects. Such compositions include, but are not limited to growth factors, cytokines, hydroxyapatite/tricalcium phosphate particles (HA/TCP), gelatin, poly-L-lysine, and collagen.

The present invention encompasses a method of treating a disorder amenable to cell therapy comprising administering to the affected subject a therapeutically effective amount of the cells of the invention.

In one embodiment, the cells are obtained and cultured as described herein in order to derive and store the cells for therapeutic uses using cell therapy should the subject require, for example, disease therapy, tissue repair, transplantation, treatment of a cellular debilitation, or treatment of cellular dysfunctions in the future.

In another embodiment of the invention, cells derived from a subject are directly differentiated in vitro or in vivo to generate differentiating or differentiated cells without generating a cell line. These cells are useful in medical and biological research and in the treatment of disease by providing cells for use in cell therapy, e.g., allogeneic cell therapy.

The adipose tissue stem cells and adipose tissue-derived cells generated by the above-mentioned techniques are utilized in research relating to cell biology, drug discovery, and in cell therapy, including but not limited to production of cells for the treatment of various diseases, disorders, and conditions, in addition to wound healing. In one aspect, they are useful in enhancing wound healing in diabetic patients. They are also useful for treating other wounds and injuries, as well as diseases, disorders, and conditions such as burns, skin aging, in addition to the uses for diabetic wound healing described herein.

Such cell therapy methods encompass the use of the cells of this invention in combination with growth factors or chemokines such as those inducting proliferation, lineage-commitment, or genes or proteins of interest. Treatment methods may include providing stem or appropriate precursor cells directly for transplantation where the tissue is regenerated in vivo or recreating the desired tissue in vitro and then providing the tissue to the affected subject.

The composites and/or cells of the present invention can be used as a vehicle for the in situ delivery of biologically active agents. The biologically active agents incorporated into, or included as an additive within, the composite of the subject invention can include, without limitation, medicaments, growth factors, vitamins, mineral supplements, substances used for the treatment, prevention, diagnosis, cure or mitigation of disease or illness, substances which affect the structure or function of the body, or drugs. The biologically active agents can be used, for example, to facilitate implantation of the composite or cell suspension into a subject to promote subsequent integration and healing processes. The active agents include, but are not limited to, antifungal agents, antibacterial agents, anti-viral agents, anti-parasitic agents, growth factors, angiogenic factors, anesthetics, mucopolysaccharides, metals, cells, and other wound healing agents. Because the processing conditions can be relatively benign (physiological temperature and pH), live cells can be incorporated into the composite during its formation, or subsequently allowed to infiltrate the composite through tissue engineering techniques.

In one aspect, the cells of the invention are useful for treating diabetic retinopathy.

According to an embodiment, a formulation of the invention contains an antimicrobial agent. The antimicrobial agent may be provided at, for example, a standard therapeutically effective amount. A standard therapeutically effective amount is an amount that is typically used by one of ordinary skill in the art or an amount approved by a regulatory agency (e.g., the FDA or its European counterpart).

The composition of the invention can further comprise additional therapeutic agents, alone or in combination (e.g., 2, 3, or 4 additional additives). Examples of additional agents include but are not limited to: (a) antimicrobials, (b) steroids (e.g., hydrocortisone, triamcinolone); (c) pain medications (e.g., aspirin, an NSAID, and a local anesthetic); (d) anti-inflammatory agents; and (e) combinations thereof.

The present invention provides methods for administering ASCs and their progeny to subjects in need thereof. In one aspect, the ASCs have been pretreated to differentiate into a precursor cell of interest or into a fully differentiated state. In another aspect, populations of ASCs can be treated with more than one type of differentiation inducing agent or medium, or a combination of agents, which induce more than one type of differentiation. In another aspect, separate populations of ASCs, that have been pretreated with cell differentiation-inducing compounds, or no treatment at all, can be co-administered to a subject. Co-administration of different groups of cells does not necessarily mean that the ASC populations are actually administered at the same time or that the populations are combined or administered in the same composition. The invention further provides compositions and methods for administering ASCs to subjects and then inducing the ASCs to differentiate in vivo by also administering cell differentiation-inducing agents to the subject. In one aspect, the subject is a human. When more than one differentiation agent or compound is used to induce cells along a particular cell pathway, or when additional agents are also used to induce some of the cells to differentiate along a second pathway, the various agents need not be provided at the same time. Various compounds and growth factors can be used with the cells of the invention to induce or modulate differentiation or maturation.

In one embodiment, a composition comprising the cells of the invention is administered locally by injection. Compositions comprising the cells can be further combined with known drugs, and in one embodiment, the drugs are bound to the cells. The cells can be seeded onto the desired site within the tissue to establish a population. The present invention thus provides methods and compositions for delivering incredibly large numbers of ASCs, precursors, or differentiated cells derived from adipose tissue for the procedures and treatments described herein. Additionally, for diseases that require cell infusions or administration, adipose tissue harvest is minimally invasive, yields many cells, and can be done repeatedly

The present invention encompasses the preparation and use of immortalized cell lines, including, but not limited to, adipose tissue-derived cell lines capable of differentiating into at least one cell type. Various techniques for preparing immortalized cell lines are known to those of ordinary skill in the art.

In one embodiment, genes of interest can be introduced into cells of the invention. In one aspect, such cells can be administered to a subject. In one aspect, the subject is afflicted with a disease, disorder, condition, or injury. In one aspect, the cells are modified to express exogenous genes or are modified to repress the expression of endogenous genes, and the invention provides a method of genetically modifying such cells and populations. In accordance with this method, the cell is exposed to a gene transfer vector comprising a nucleic acid including a transgene, such that the nucleic acid is introduced into the cell under conditions appropriate for the transgene to be expressed within the cell. The transgene generally is an expression cassette, including a coding polynucleotide operably linked to a suitable promoter. The coding polynucleotide can encode a protein, or it can encode biologically active RNA (e.g., antisense RNA or a ribozyme). Thus, for example, the coding polynucleotide can encode a gene conferring resistance to a toxin, a hormone (such as peptide growth hormones, hormone releasing factors, sex hormones, adrenocorticotrophic hormones, cytokines (e.g., interferons, interleukins, lymphokines), a cell-surface-bound intracellular signaling moiety (e.g., cell adhesion molecules, hormone receptors), a factor promoting a given lineage of differentiation, etc.

In addition to serving as useful targets for genetic modification, many cells and populations of the present invention secrete various polypeptides. Such cells can be employed as bioreactors to provide a ready source of a given hormone, and the invention pertains to a method of obtaining polypeptides from such cells. In accordance with the method, the cells are cultured under suitable conditions for them to secrete the polypeptide into the culture medium. After a suitable period of time, and preferably periodically, the medium is harvested and processed to isolate the polypeptide from the medium. Any standard method (e.g., gel or affinity chromatography, dialysis, lyophilization, etc.) can be used to purify the hormone from the medium, many of which are known in the art.

In other embodiments, cells (and populations) of the present invention secreting polypeptides can be employed as therapeutic agents. Generally, such methods involve transferring the cells to desired tissue, either in vitro or in vivo, to animal tissue directly. The cells can be transferred to the desired tissue by any method appropriate, which generally will vary according to the tissue type.

In other embodiments, therapeutic agents, including, but not limited to, cytotoxic agents, anti-angiogenic agents, pro-apoptotic agents, antibiotics, hormones, hormone antagonists, chemokines, drugs, prodrugs, toxins, enzymes, or other agents may be used. Additionally, they may be used as adjunct therapies when using the liposome complexes described herein. Drugs useful in the invention may, for example, possess a pharmaceutical property selected from the group consisting of antimitotic, antikinase, alkylating, antimetabolite, antibiotic, alkaloid, anti-angiogenic, pro-apoptotic agents, and combinations thereof. In one aspect, the drug or agent is encapsulated into a liposome of the invention.

The peptides and proteins of the present invention may be purchased or readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide. Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C4-, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation,” a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones, or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

The present invention also provides for homologs of proteins and peptides. Homologs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, 10 or more conservative amino acid changes typically have no effect on protein function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Homologs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substantially pure protein or peptide obtained as described herein may be purified by following known procedures for protein purification, wherein an immunological, enzymatic, or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

### Amino Acid Substitutions

In certain embodiments, the disclosed methods and compositions may involve preparing peptides with one or more substituted amino acid residues. In various embodiments, the structural, physical and/or therapeutic characteristics of peptide sequences may be optimized by replacing one or more amino acid residues.

In one embodiment, the invention encompasses the substitution of a serine or an alanine residue for a cysteine residue in a peptide of the invention. Support for this includes what is known in the art. For example, see the following citation for justification of such a serine or alanine substitution: Kittlesen et al., 1998 Human melanoma patients recognize an HLA-A1-restricted CTL epitope from tyrosinase containing two cysteine residues: implications for tumor vaccine development J Immunol., 60, 2099-2106.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art. For example, one can make the following isosteric and/or conservative amino acid changes in the parent polypeptide sequence with the expectation that the resulting polypeptides would have a similar or improved profile of the properties described above:

Substitution of Alkyl-Substituted Hydrophobic Amino Acids:

including alanine, leucine, isoleucine, valine, norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of Aromatic-Substituted Hydrophobic Amino Acids:

including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3- or 4-chlorophenylalanine, 2-,3- or 4-methylphenylalanine, 2-,3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′,-3′,- or 4′-methyl-2, 3 or 4-biphenylalanine, and 2- or 3-pyridylalanine.

Substitution of Amino Acids Containing Basic Functions:

including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of Acidic Amino Acids:

including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of Side Chain Amide Residues:

including asparagine, glutamine, and alkyl or aromatic substituted derivatives of asparagine or glutamine.

Substitution of Hydroxyl Containing Amino Acids:

including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.) Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

One of ordinary skill in the art will appreciate that the amount of agent used can vary, depending on such factors as the source of the cells used, the age of the subject, the health of the subject, and the agent used. In one aspect, cells are contacted with an agent at a concentration ranging from about 0.1 μg/ml to about 10 mg/ml. In one aspect, the range is from about 1.0 μg/ml to about 1.0 mg/ml. In one aspect, about 1.0 μg/ml of enhancing agent is used.

Pharmaceutical compositions comprising the present cells, conditioned medium, or compounds are administered to an individual in need thereof by any number of routes including, but not limited to, intraocular, intravitreal, subretinal, topical, oral, intravenous, intramuscular, intra-arterial, intramedullary, intrathecal, intraventricular, transdermal, subcutaneous, intraperitoneal, intranasal, enteral, topical, sublingual, or rectal means.

The present invention is also directed to pharmaceutical compositions comprising the peptides of the present invention. More particularly, such compounds can be formulated as pharmaceutical compositions using standard pharmaceutically acceptable carriers, fillers, solublizing agents and stabilizers known to those skilled in the art.

The invention also encompasses the use pharmaceutical compositions of an appropriate compound, homolog, fragment, analog, or derivative thereof to practice the methods of the invention, the composition comprising at least one appropriate compound, homolog, fragment, analog, or derivative thereof and a pharmaceutically-acceptable carrier.

The pharmaceutical compositions useful for practicing the invention may be administered to deliver a dose of between 1 ng/kg/day and 100 mg/kg/day. Pharmaceutical compositions that are useful in the methods of the invention may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the invention.

Compounds which are identified using any of the methods described herein may be formulated and administered to a subject for treatment of the diseases disclosed herein.

The invention encompasses the preparation and use of pharmaceutical compositions comprising a compound useful for treatment of the conditions, disorders, and diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the invention may be prepared, packaged, or sold in formulations suitable for, including, but not limited to, intravitreal, oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations. A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Examples of antimicrobial agents that can be used in the present invention include, but are not limited to, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, cikprofloxacin, doxycycline, ampicillin, amphotericine B, ketoconazole, fluconazole, pyrimethamine, sulfadiazine, clindamycin, lincomycin, pentamidine, atovaquone, paromomycin, diclarazaril, acyclovir, trifluorouridine, foscarnet, penicillin, gentamicin, ganciclovir, iatroconazole, miconazole, Zn-pyrithione, and silver salts, such as chloride, bromide, iodide, and periodate.

In one embodiment, the biologically active agents or compounds can first be encapsulated into microcapsules, microspheres, microparticles, microfibers, reinforcing fibers and the like to facilitate mixing and achieving controlled, extended, delayed and/or sustained release and combined with the cells of the invention. Encapsulating the biologically active agent can also protect the agent against degradation during formation of the composite of the invention.

The invention also includes a kit comprising the composition of the invention and an instructional material which describes administering or using the composition. In another embodiment, this kit comprises a (preferably sterile) solvent suitable for dissolving or suspending the composition of the invention prior to administering the composition. Optionally, at least one growth factor and/or antimicrobial agent may be included in the kit.

Without further description, it is believed that one of ordinary skill in the art can, using the preceding description and the following illustrative examples, make and utilize the compounds of the present invention and practice the claimed methods. The following examples therefore, specifically point out the preferred embodiments of the present invention, and are not to be construed as limiting in any way the remainder of the disclosure.

### EXAMPLES

### Example 1

### Hyperglycemia Impairs the Regenerative Ability of Adipose-Derived Stem Cells in the Treatment of Diabetic Retinopathy

Materials and Methods—

mASC Harvest and Culture

Isolation of the stromal vascular fraction from the epididymal fat pad and culture of mASCs was performed as detailed in (Zuk). Briefly, fat pads were harvested from 9-week old Akimba and WT mice, then digested in collagenase-containing (Sigma, St. Louis, Mo.) digestion buffer for one hour at 37° C. The resulting mixture was filtered through 200-μm mesh to exclude any undigested tissue. The filtrate was centrifuged to remove remaining collagenase, and the pellet incubated with red blood cell lysis buffer (Sigma, St. Louis, Mo.) for 5 minutes at room temperature. The cell suspension was then sterile-filtered through 40-μm mesh and plated on sterile culture plates (Corning, Corning, N.Y.). Cells harvested from Akimba mice and WT mice are referred to as “diabetic” and “healthy” mASCs, respectively.

mASCs were maintained in a sterile culture hood and incubator at 37 degrees C. and 5% CO2. Growth media consisted of 10% fetal bovine serum (Hyclone, Logan, Utah) and 1% penicillin/streptomycin in Dulbecco's Modified Eagle Medium (DMEM-F12) with added glutamate and sodium bicarbonate (Life Technologies). Cells were passaged at roughly 80% confluence, and media was changed every other day.

Labeling and Intraocular Injection of mASCs

All animal studies were approved by the University of Virginia's Animal Care and Use Committee. Healthy or wild-type mice were of the C57BL/6 strain, and diabetic mice were Akimba on the C57BL/6 background. Presence of the Akita genotype was detected by RT-PCR and hyperglycemia was confirmed by taking blood glucose measurements. Healthy and diabetic mASCs were fluorescently labeled with Vybrant DiI Cell-Labeling Solution (Life Technologies, Grand Island, N.Y.) as per manufacturer's instructions, and resuspended in PBS at the appropriate concentration determined by hemocytometer. Each cell injection consisted of 10,000 mASCs suspended in 1.5 uL of PBS. Control vehicle injections consisted of 1.5 uL of phosphate-buffered saline (PBS). Cells were resuspended by pipette immediately before injection of each eye to minimize cell clumping. Male 4 week old Akimba mice were anesthetized with ketamine/xylazine injected intraperitoneally and proparacaine applied topically to the eyes just prior to injection. mASCs were injected through the pars plana into the vitreous using a 33-gauge Hamilton syringe. A total of 12 Akimba mice were injected in this manner with mASCs in one eye and a contralateral PBS control injection in the other eye. In six of these mice, healthy mASCs were used, and in the other six diabetic mASCs were used.

### Retinal Whole-Mounting and Immunostaining

Treated mice were left for four weeks post-injection before harvesting retinae. Mice were euthanized with carbon dioxide, followed immediately by cardiac perfusion-fixation by cutting the right atrium and injecting the left ventricle with 10 mL of 4% paraformaldehyde (PFA). Intact eyes were then removed and fixed by submersion in 4% PFA for 10 minutes. After rinsing the eyes with PBS, retinae were isolated and whole-mounted on gel-coated slides. Retinae were then permeabilized with 1 mg/mL digitonin (MP Biomedical, Solon, Ohio) for 1 hour and stained with mouse monoclonal AlexaFluor-647 (Life Technologies) overnight.

Imaging & Image Analysis: 10×, 20×, and 60× image stacks were taken on a Nikon confocal laser scanning microscope. 10× stacks were flattened and tiled into whole-retina montages using ImageJ. Vessel length of whole retinae was calculated digitally by skeletonization in ImageJ and subsequent pixel count of the skeletonized vasculature in MATLAB using a custom analysis routine. Vascular density is equal to the ratio of vessel length to whole retina area.

Labeled mASCs were counted in three representative 20× image stacks per retina by blinded observers. Each field of view was taken at the same distance from the optic nerve.

### Monte Carlo Simulation

A Monte Carlo simulation was created in MATLAB (version 2013a) to determine the random placement of mASCs on retinal vasculature. The simulation used binary images of fluorescent retinal vasculature micrographs (20× magnification) to generate a matrix of coordinates for the retinal vasculature. For each micrograph, a random matrix of mASC coordinates was generated. Identical retinal vasculature coordinates and mASC coordinates were counted to determine the probability of mASCs randomly contacting retinal vasculature after random distribution. The simulation was looped 1000 times for more accurate probability calculations. Utilizing Delaunay triangulation, the simulation also created a histogram to visualize the probability distribution of the distance of randomly simulated mASCs from retinal vasculature.

### Angiogenesis Secretome

Conditioned media samples were obtained from 3 populations each of healthy and diabetic mASCs, taken from wild type and Akimba mice of the same age, respectively. All cells were simultaneously passaged to P4, counted, plated with fresh media, and allowed to incubate at 37° C. for 24 hours before collection of conditioned media. These samples were run on a Mouse Proteome Profiler Angiogenesis Array (RnD Systems ARY015), which tested for 53 angiogenic factors. X-ray film captured chemiluminescence from each dot, which was proportional to the amount of factor bound. Relative expression levels were obtained from densitometry analysis (ImageJ) of x-ray film spots. Finally, angiogenic factor levels were normalized to positive control and cell count.

### Cell Proliferation & Apoptosis Assays

mASCs from 3 healthy 9 week old wildtype and 3 9 week old diabetic Akimba animals were plated on glass coverslips in 12-well dishes at a density of 4000 cells/cm2 and allowed to adhere for 24 hours (Cold Spring Harbor proceedings). To measure cell proliferation in each population of mASCs, mASCs were incubated with 5-ethynyl-2′-deoxyuridine (EdU) for 12 hours and subsequently stained following the manufacturer's instructions. To measure apoptosis, the terminal deoxynucleotidyl transferase dUTP nick end labeling (TUNEL) method was used. Fluorescence intensity was measured for each coverslip using confocal microscopy, taking image stacks with a 3 um spacing.

### Statistical Analysis

In vivo studies were analyzed for numerical differences using paired t-tests comparing left and right eyes for 6 healthy and 6 diabetic animals. All in vitro mASC experiments were performed with 3 healthy and 3 diabetic animals, and significance was determined using a student's t-test. Statistical tests including t-tests, paired t-tests, Mann-Whitney Rank Sum tests, and ANOVAs were performed in SigmaStat (Systat Software Inc, San Jose, Calif.). Statistical significance was determined using a maximum p-value of 0.05, or smaller if indicated.

### Supplementary Information for Example 1

### Measurements of Oxygen Consumption and Mitochondrial Function (Methods):

Oxygen consumption rate (OCR) was measured using a Seahorse XF-24 Flux Analyzer (Seahorse Biosciences, Billerica, Mass.), as previously described 1. mASCs were seeded in a Seahorse 24-well tissue culture plate at a density of 3.0×10⁴ cells/well in normal growth media for 24 hours prior to the assay. On the day of the assay the media was changed to unbuffered DMEM and equilibrated at 37° C. Oligomycin (2 μM), FCCP (1 μM), and Rotenone and Antimycin A (1 μM and 10 μM) were injected sequentially during the assay as indicated. Each OCR measurement represents the average of a 3 minute reading every 10 min, with 3 min of mixing and 4 min of wait time per cycle. 3-4 wells were used for each experimental group.

### Cell Force Measurements

Measurements of total cell contractile force were carried out as described in (Yang, Fu et al. 2011). Briefly, mASCs were plated at a density of 5000 cells/cm2 on micropost substrate arrays. After allowing to adhere for 7 minutes, non-adherent cells were washed off and plates were inspected to ensure a majority of cells were occupying isolated positions without cell-cell contacts. Cell culture medium was replaced and cells allowed to continue adhering overnight. Cells were fixed using a 4% paraformaldehyde solution for 20 minutes and then stained with Alexa Fluor 488 Phalloidin (Molecular Probes) to observe the f-actin cytoskeleton. Confocal images of micropost “top” positions and cell outlines were analyzed in MATLAB to generate force maps and calculate total cell force on a cell-by-cell basis.

### Results

### Example 1

### Regenerative Potential

Using the Akimba murine model of diabetic retinopathy, we tested whether hyperglycemia alters mASCs ability to rescue diseased microvasculature in the Akimba retina. The Akimba retina demonstrates several hallmarks of proliferative diabetic retinopathy in humans, including retinal thinning and severe vascular pathology such as capillary dropout, nonperfusion, retinal neovascularization, retinal edema, and microaneurysms (Rakoczy). mASCs used for treatment were harvested from either 9 week old hyperglycemic Akimba mice or wild-type mice both on a C57BL/6 background. Each mouse received injected mASCs in one eye (either healthy or diabetic) and a contralateral PBS vehicle control. Confocal images of isolectin-stained retinae and DiI-labeled injected mASCs are shown in FIGS. 1A and C. The greater loss of capillaries in diabetic mASC-treated retinae compared to healthy mASC-treated retinae is visually apparent. To quantify this difference in treatment outcome, vascular density was calculated digitally by the ratio of total vessel length to total retinal area. The change in vascular density of mASC-injected eyes minus that of control eyes is shown graphically in FIG. 1. To correct for variability in disease severity between individual mice, all analysis on vascular density was performed by comparing the mASC-treated retina to the control PBS-injected retina in the same mouse. A majority of eyes treated with diabetic mASCs had a lower vascular density compared to contralateral controls (FIGS. 1A and B). When considering the overall change of vascular density, diabetic mASC-treated eyes displayed both increases and decreases in vascular density with no meaningful trend. Meanwhile, those treated with healthy mASCs showed a significant increase in vascular density relative to contralateral controls (FIGS. 1C & D). These data indicate that diabetic mASCs are not as effective as healthy mASCs in promoting revascularization in the Akimba retina.

### Perivascular Incorporation

Observing reduced vessel density and qualitatively impaired healing due to diabetic mASCs compared to healthy mASCs may be due in part to the ability of transplanted cells to incorporate into host retinal vasculature. DiI-labeled mASCs were counted from representative 20× confocal image stacks. Each counted cell was sorted as either perivascular or non-perivascular for eyes treated with healthy and diabetic mASCs, shown in FIG. 2. An equal number of both types of cells were injected, but a greater number of total mASCs incorporated into the retina for healthy mASCs than diabetic mASCs. Furthermore, healthy mASCs were found in perivascular positions more often than non-perivascular positions. Diabetic mASCs were found in relatively equal abundance both in perivascular and non-perivascular positions. It is important to note that the cells themselves are large compared to the vessels and in some cases the spacing between the vessels.

To determine whether the injected mASCs preferentially took up perivascular positions, or fell there by chance, we carried out a Monte Carlo simulation on each 20× field of view. This stochastic simulation provided an estimate of the number of cells for each given field that one would expect to find in contact with a vessel by chance alone. In nearly all cases, the observed number of cells in contact with vessels was within the 95% confidence interval predicted by the Monte Carlo simulation to be found within perivascular distance by chance (FIG. 3). While the observed total number of cells between healthy and diabetic populations found residing in the retina was significantly higher for healthy mASCs, any difference in tendency to assume perivascular positions cannot be determined.

mASC Viability and Apoptosis Analysis

Relative rates of proliferation and apoptosis between healthy and diabetic mASCs were quantified in-vitro with cultured mASCs. Proliferation frequencies were measured using an EdU-incorporation assay (Life Technologies etc). We found that diabetic mASC proliferation activity was 77±5% that of healthy mASCs (FIG. 4A). Diabetic mASC apoptosis activity was 121±3% that of healthy mASCs as determined by a TUNEL assay (FIG. 4B).

### MASC Cellular Bioenergetics

An analysis of the two-mASC populations' bioenergetic profiles was then performed using the Seahorse instrument to determine whether differences in cellular metabolism may account for their differing treatment efficacy and function in vivo. Specifically, we assayed mitochondrial bioenergetics in whole cells by measuring oxygen consumption over time following the sequential addition of the ATP synthase inhibitor oligomycin, the mitochondrial uncoupler FCCP, and the complexes I and III inhibitors rotenone and Antimycin A1. These data revealed that diabetic and non-diabetic mASCs had comparable rates of basal cellular respiration, ATP-dependent respiration, spare respiratory capacity, uncoupled respiration, and non-mitochondrial respiration (Example 1, Supplementary FIG. 1).

### Angiogenesis Factor Secretome

To investigate a possible paracrine-signaling basis for the differences in revascularization potential of healthy and diabetic mASCs, levels of secreted angiogenic factors were measured via high-throughput ELISA. Conditioned media samples were collected from three populations each of healthy and diabetic mASCs. Each sample was run on a separate array under identical conditions, which enabled us to calculate a relative abundance of each angiogenesis factor by comparing arrays after normalizing to a positive control. Since healthy mASCs were found to proliferate faster than diabetic mASCs in culture, angiogenesis factor levels were also normalized to cell number as counted immediately prior to collection of conditioned media. We found 5 angiogenic factors that were secreted at significantly higher levels by healthy mASCs than by diabetic mASCs, namely IGFBP-2, IGFBP-3, MCP-1, osteopontin, and SDF-1 (FIG. 5). The remaining factors of the 11 analyzed were secreted at similar levels by healthy and diabetic mASCs, and none were secreted at higher levels by diabetic mASCs.

### Discussion

### Example 1

ASC therapy holds promise for many debilitating and increasingly common microvascular disorders due to their regenerative potential. The ability of ASCs to secrete a wide range of cytokines which have paracrine and autocrine effects both prolongs their regenerative effect in vivo and allows them to respond in a more specific manner than treatment with growth factors or small molecules (cite). Furthermore, isolation of ASCs has several advantages over other cell types due to the relative abundance of adipose tissue and relatively painless extraction procedure (cite). The fact that ASCs can be rapidly isolated at high enough quantities for treatment without culture and expansion holds great implications for autologous treatment options. Thus, it is important to assess whether or to what extent ASCs isolated from a diabetic source have compromised treatment efficacy and function.

In this study, we first demonstrated that mASCs from diabetic sources are not as effective as those from healthy sources in promoting revascularization in diabetic retinopathy. In addition to highlighting the effect of diabetes on mASCs treatment efficacy in vivo, this experiment corroborated our group's previous findings that healthy ASCs regenerate damaged retinal microvasculature. In contrast to the current study, these previous results were obtained using the oxygen-induced retinopathy model in immunosuppressed mice, and with ASCs isolated from only non-diabetic human adipose tissue (Mendel). In an effort to explain the observed decreased treatment efficacy when using ASCs from diabetic sources, we carried out an analysis of mASC incorporation in vivo, and an in vitro functional analysis. We found that a higher ratio of healthy mASCs incorporated into the retina than diabetic mASCs. mASCs from diabetic sources were additionally found to undergo proliferation less frequently and apoptosis more frequently, in accordance with other functional studies using diabetic mASCs. Finally, mASCs from diabetic sources secreted lower levels of angiogenesis-promoting factors.

The observed differences in mASCs isolated from healthy and diabetic sources have implications for the use and study of stem cells beyond the model disease in this study. Based on the fact that diabetic mASCs are impaired in their regenerative ability to the point that their use did not elicit a predictable and positive response in vivo, an autologous approach to ASC therapy in diabetic patients cannot be recommended without considering and possibly correcting for these differences. Furthermore, the greater levels of angiogenesis factor secretion in healthy mASCs and concomitant increase in microvascular regeneration supports the notion that ASCs act on surrounding tissues through paracrine activity.

It is interesting to note that while diabetic mASCs showed decreased secretion of these factors as well as functional impairment, the impairment was not universally seen in all aspects of cell function. Specifically, metabolic capacity, mitochondrial function, and contractile force generation were identical in healthy and diabetic cells, suggesting diabetes-associated hyperglycemia had no lasting effect on these functions (Supplementary FIG. 1). While others have reported significant hyperglycemia-induced changes in metabolism in other cell types such as bovine retinal pericytes (cite), these studies were all performed using high-glucose media. The fact that we saw no metabolic differences after culturing cells to P4 indicates that these differences were likely transient, in contrast to angiogenesis factor secretion, which was persistent. Possible mechanisms for this relative persistence might be epigenetic modifications in response to hyperglycemia, which are selective for cell cycle and growth factor regulation (cite).

Pericytes belong to the vascular smooth muscle cell lineage, and are known to communicate with endothelial cells through direct cell-cell contact, and regulate blood flow through contraction and relaxation (Zlokovic). The fact that healthy and diabetic mASCs both were not found preferentially in contact with blood vessels, and showed similar contractile force generation yet different effects on the microvasculature suggests that their different effects on vessel density was not attributed to a difference in their ability to exert mechanical force.

As a potential therapy for diabetic retinopathy, it is important to know how ASCs behave in the retina. Examining the number and placement of mASCs found in retinae from our study with the Akimba mouse can provide valuable insight into this. Mesenchymal stem cells (MSCs) are thought to be closely connected with perivascular cells (Sharpe), and ASCs have been shown to originate from perivascular MSCs which reside in great numbers in adipose tissue (Cai). This would suggest that ASCs might exert their vascular-stabilizing effects by taking up perivascular positions. However, our results combining in vivo data with a stochastic model of mASC placement indicate that the number of mASCs in contact with vessels likely incorporated in those locations by chance rather than preferentially residing there by chemotaxis or other homing mechanisms. The fact that there are significantly more healthy mASCs than diabetic mASCs residing in the retina after four weeks regardless of perivascular placement is likely due to a combination of several factors such as increased rates of apoptosis as well as increased migration capabilities of diabetic mASCs as demonstrated by others (Cianfarani, Fadini).

In summary, we have shown that ASCs taken from diabetic sources impairs their ability to regenerate the microvasculature in diabetic retinopathy, and that use of autologously-derived ASCs from diabetic patients would not be effective. Furthermore, hyperglycemia causes distinct changes in ASC function, but only to certain aspects related to cell cycle and growth factor regulation. Finally, our results support the notion that ASCs promote vascular regeneration through paracrine activity rather than direct cell-cell contact. To improve ASC therapy for diabetic retinopathy as well as other diabetic complications, it is critical to better understand their mechanism of action in vivo. Future work may focus on describing the means by which ASCs migrate towards and incorporate into the retina and their activity in vivo, as well as the mode of diabetes-induced changes in ASC function.

### Bibliography

### Example 1

1. Kenwood, B. M. et al. Identification of a novel mitochondrial uncoupler that does not depolarize the plasma membrane. Molecular Metabolism, doi:http://dx.doi.org/10.1016/j.molmet.2013.11.005.
2. Cianfarani, F. et al. Diabetes impairs adipose tissue-derived stem cell function and efficiency in promoting wound healing. Wound repair and regeneration: official publication of the Wound Healing Society [and] the European Tissue Repair Society 21, 545-553, doi:10.1111/wrr.12051 (2013).
3. U.S. Patent Application Publication No. US 2007/0154465 A1, Kharazi, et al., “Stem Cell Therapy for Retinal Disease”, Jul. 5, 2007.
4. U.S. Patent Application Publication No. US 2003/0125293 A1, Cheatham, et al., “Adipose Tissue-Derived Stromal Cells for the Repair of Corneal and Intra-Orbital Defects and Uses Thereof”, Jul. 3, 2003.
5. U.S. Patent Application Publication No. US 2006/0147430 A1, Sayre, et al., “Adipose-Derived Stem Cells for Tissue Regeneration and Wound Healing”, Jul. 6, 2006.
6. U.S. Patent Application Publication No. US 2008/0213235 A1, Katz, et al., “Adipose Tissue Stem Cells, Perivascular Cells ad Pericytes”, Sep. 4, 2008 (UVA Ref Katz-Population).
7. Amos, P., et al., “IFATS Series: The Role of Human Adipose-Derived Stromal Cells in Inflammatory Microvascular Remodeling and Evidence of a Perivascular Phenotype”, Stem Cells, 2008 October: 26 (10): 2682-2690.
8. Sieczkiewicz, G., et al., “TGF-β1 signaling controls retinal Pericyte contractile protein expression”, Microvascular Research 66 (2003), 190-196.
9. Gerhardt, H., et al., “Endothelial-pericyte interactions in angiogenesis”, Cell Tissue Research (2003) 314: 15-23.
10. Yang, Z., et al., “Amelioration of diabetic retinopathy by engrafted human adipose-derived mesenchymal stem cells in streptozotocin diabetic rats”, Graefes Arch. Clin. Exp. Opthalmol., (2010), 248: 1415-1422.
11. U.S. Patent Application Publication No. US 2012/0148546 A1, Dar-Oaknin, et al., “Pericyte Progenitor Cells and Methods of Generating and Using Same”, Jun. 14, 2012; provisional app. filed 2009; PCT published in 2011.
12. Mendel, T., et al., “Pericytes Derived from Adipose-Derived Stem Cells Protect against Retinal Vasculopathy”, PLOS One, May 2013, Vol. 8, Issue 5, e65691, 1-11.
13. Armulik, A., et al., “Endothelial/Pericyte Interactions”, Circ. Res., 2005; 97:512-523.

### Example 2

### Pericytes Derived from Adipose-Derived Stem Cells Protect Against Retinal Vasculopathy

It is disclosed in Example 2 that:

1) human adipose-derived stem cells injected intravitreally can integrate with retinal vasculature and differentiate into pericytes;

2) injection of the cells/treatment can accelerate recovery from oxygen-induced retinopathy and protect against oxygen-induced retinopathy;

3) pre-treatment of the human adipose-derived stem cells with TGF-β elicit a response in the integrated cells analogous to what was known to occur for endogenous pericytes treated with TGF-β (i.e., increased pericyte marker expression); and

4) use of the cells in a model of diabetic retinopathy can prevent retinal capillary dropout.

This example is based on Mendel et al., 2013, (PLoS ONE 8(5): e65691. doi:10.1371/journal.pone.0065691), which is included as Appendix A and which is incorporated by reference in its entirety herein.

Human adipose derived stem cells (hASCs) are perivascular cells easily isolated from adipose tissue and postulated to have a direct role in adipose vascular support and repair. We assayed the ability of intravitreally injected hASCs to migrate to the retina and stabilize retinal microvasculature, by utilizing a well-characterized murine OIR model in which central retinal vasculature is ablated with hyperoxic atmospheric conditioning from post-natal day 7 (P7) to P12, followed by revascularization of hypoxic central retina upon return to normal room air at P12.

hASCs were harvested from patients undergoing elective plastic surgery and subsequently passaged serially in culture, as previously described. Unconditioned and unsorted passage 5 hASCs were labeled with 1,1′-dioctadecyl-3,3,3′3′-tetramethylindocarbocyanine perchlorate (DiI), suspended in phosphate buffered saline (PBS), and injected intravitreally into immunocompromised NOD SCID mice (Charles River) at P12 upon removal from hyperoxia. PBS was injected into the vitreous of the contralateral eye to serve as a carrier control.

Immunocompromised mice were used to avoid a non-specific inflammatory reaction to xenografted hASCs. Following retinal harvest and endothelial staining with isolectin, DiI-labeled hASCs were observed to have successfully traversed through the vitreous and integrated into the retinal vasculature ten days later (Example 2, FIG. 1a). Retinas of hASC injected mice harvested eight weeks later contained an average of 308 DiI labeled cells, or 3.08% (2.08-4.07 95% CI) of the 10,000 injected cells; comparable to stem cell engraftment efficiencies observed in other model systems. Of the cells engrafted into the retina, 85.6% (82.8-88.7% 95% CI) were found in physical contact with retinal microvessels, many adopting pericyte-like abluminal locations, and phenotypic wrapping around vessels (Example 2, FIG. 1b, c). Retinas treated with hASCs at P12 and harvested at P14 demonstrated accelerated revascularization of the previously ablated central retina, compared with contralateral PBS injected controls (Example 2, FIG. 1 d-f), indicating injected hASCs may aid in stabilizing retinal angiogenic endothelial cells. To determine if hASCs could also protect retinal microvasculature from initial endothelial loss from hyperoxic ablation, hASCs were intravitreally injected at either P2 or P5. The mice placed into a hyperoxic environment at P7, and retinas harvested at P12 upon removal from hyperoxia. Surprisingly, hASC treated retinas exhibited a profoundly reduced avascular area compared to contralateral controls (Example 2, FIG. 1 h-j), suggesting that injected hASCs may assist in stabilizing retinal microvasculature that is otherwise acutely unstable. In hASC injected eyes, higher vascular length density was seen in retinal sub-fields containing labeled cells as compared sub-fields without cells, and this could account in part for the observed results (Example 2, Supplementary FIG. 2).

Human retinal pericytes are known to express VEGF165b in the developing retina, and this paracrine signal is thought to competitively inhibit the VEGF 165 angiogenic variant. This potentially stabilizes retinal microvasculature, by protecting against the destabilizing effects from VEGF165 binding on endothelial cells. Notably, VEGF165b levels are known to decrease following OIR exposure, with corresponding increases in VEGF. Considering the strong impact hASCs appear to exert in acute retinopathy and their pericyte like morphology in vivo, we asked if our results might be explained in part by altered VEGF165b levels. Similar to human pericytes, we find hASCs strongly express VEGF165b in culture (Example 2, Supplementary FIG. 3a). We then assessed retinal expression of VEGF165b at P17 in eyes that were injected intravitreally at P5 with either hASC, and PBS carrier, and then exposed to OIR from P7 to P12. At P17 there were significantly increased levels of VEGF165b in retinas treated with hASCs as compared to PBS injected controls (Example 2, Supplementary FIG. 3b). In total, our OIR results suggest intravitreal injection of hASCs can provide functional pericytes to hypoxic retina where they likely exert microvascular stabilization through both direct contact and paracrine interaction with endothelial cells; both mechanisms characteristic of native retinal pericytes.

Although a functional pericyte phenotype for hASCs is suggested by their morphology, microvascular location, and ability to stabilize vasculature, we sought to further explore their expression of characteristic pericyte markers. Smooth muscle actin (SMA), NG2 chondroitin sulphate proteoglycan (NG2), platelet derived growth factor receptor beta (PDGFRβ), and regulator of G-protein signaling 5 (RGS5) frequently designate a pericyte phenotype, although their expression is dependent upon microvascular context in vivo. We found unconditioned passage 5 hASCs richly express SMA, NG2, and PDGFRβ in culture (Example 2, FIG. 2 a, c, e) and by flow cytometry (Example 2, FIG. 2 b, d, f). Additionally, unconditioned hASCs injected intravitreally into NOD SCID mice at P12, upon removal from hyperoxia in the OIR model, trafficked to the retina, integrated with retinal microvessels, and continued to express defined pericyte markers including SMA, NG2, and RGS-5 when harvested six to eight weeks after injection (Example 2, FIG. 2 g-i).

With both in vivo and in vitro evidence suggesting that unconditioned hASCs can differentiate into functional retinal pericytes with only microenvironmental cues and help support retinal microvasculature, we sought to pre-treat hASC cultures with TGFβ to further enrich the differentiation and function of the heterogeneous stem cell harvest product. TGFβ, a factor known to induce differentiation of other stem cell lineages, is required to maintain retinal vascular barrier function, is implicated in mural cell differentiation, and has been shown to influence VEGF post-transcriptional splicing, directly impacting VEGF 165b production. These attributes could contribute to our previous in vivo OIR findings. hASCs were pre-treated in culture for 48 hours with 1 ng/ml TGFβ, and following subsequent harvest were assayed both in vivo and in vitro using standard assays of retinal pericyte function to determine potential enhancement of pericyte markers expression, functional contractility, and ability to stabilize endothelial cells.

Compared to unconditioned hASCs, TGFβ conditioned hASCs expressed more SMA in culture and by flow cytometry (Example 2, FIG. 3a) while β actin control did not increase with TGFβ treatment. Further, hASCs that were treated with TGFβ demonstrated enhanced contractile ability, as measured by increased silicone substrate wrinkling (Example 2, FIG. 3c). Fewer endothelial cells contacting TGFβ conditioned hASCs were observed undergoing mitosis than endothelial cells contacting unconditioned hASCs (Example 2, FIG. 3d). Finally, we sought to confirm the in vitro evidence that TGFβ enhances the pericyte phenotype with a modified version of our in vivo OIR vascular protection assay. P5 NOD SCID pups were injected with unconditioned hASCs in one eye and TGFβ conditioned hASCs in the contralateral eye. Retinas harvested and stained at P12 upon removal from hyperoxia revealed eyes injected with TGFβ conditioned hASCs had enhanced microvascular protection compared to contralateral eyes treated with unconditioned hASCs (Example 2, FIG. 3e, f). Considering the consistent in vitro findings from flow cytometry, cellular contractility, and endothelial stabilization, as well as in vivo confirmation of endothelial stabilization, these results suggest cultured hASCs contain a subpopulation of pericyte precursor cells, which may be further enhanced or selected for with TGFβ conditioning.

Having established a functional retinal pericyte phenotype for hASCs, we next sought to determine their potential application for treatment of diabetic retinopathy. While no single animal model captures all clinical phenotypes of human diabetic retinopathy, the recently published Akimba model features large-scale retinal capillary dropout in an immunocompetent diabetic mouse; findings that are highly characteristic of severe forms of human disease. Briefly, diabetic Ins2^{Akita} mice were crossed with Kimba mice that feature rhodopsin promoter driven human VEGF 165, which pulses at P10 and destabilizes retinal capillary beds. The resultant Akimba pups exhibit profound retinal capillary dropout by 8 weeks of life. Of note, these mice are fully immunocompetent, precluding the use of xenografted hASCs, instead necessitating use of heterologous murine adipose derived stem cells (mASCs).

To test the hypothesis that mASCs can stabilize Akimba retinal microvasculature, we injected mASCs that were pre-treated with TGFβ and pre-labeled with DiI prior to injection into P9 pup eyes. As before, PBS was injected as a carrier control in the contralateral eye. Prior to injection, SMA expression was verified in cultured mASCs at a level comparable to that observed with hASCs (data not shown). At eight weeks of age, after verifying diabetic status of each mouse (data not shown), mice were imaged in vivo with fluorescein angiography using customized Cantor Nissel contact lenses and the Heidelberg Spectralis Retinal Imager (Example 2, FIG. 4a, c). Subsequently, retinas were harvested, whole-mounted, and stained with lectin (Example 2, FIG. 4b, d), revealing a substantial reduction in area of capillary dropout in retinas treated with mASCs compared to contralateral controls (Example 2, FIG. 4e).

It was also demonstrated that pretreating or conditioning ASCs with TGF-β stabilizes preretinal neovascularization (Example 2, Supplementary FIG. 4).

Diabetic retinopathy, a disease that proceeds from initial pericyte degeneration, is particularly well suited to benefit from stem cell therapy. Groundbreaking work has previously demonstrated that intravitreally injected bone marrow-derived stem cells can incorporate into the retina, though predominantly as endothelial cells and microglia. Exogenously injected hASCs have also been shown in other model systems to differentiate into pericyte lineages, in support of our current finding. Our current results, however, are the first to demonstrate rigorously long-term stem cell differentiation and incorporation within the retina into the crucial degenerating cell type in diabetic retinopathy: the pericyte. They are also the first to establish that an injection of exogenous adult stem cells differentiated along a pericyte lineage can functionally protect retinal microvessels against profound capillary dropout in a novel model of diabetic retinopathy. Our results suggest ASCs likely stabilize retinal microvasculature through both direct contact and paracrine signaling, including VEGF165b, similar to native pericytes. Additionally, ASCs exhibit enhanced pericyte capabilities when conditioned with TGFβ.

Further work is clearly needed to illuminate the dynamic control of retinal vessel formation and degeneration in diabetic retinopathy, especially as pertains to the observed functional effects of our injected cells. Harvesting the full treatment potential of ASC-pericytes may require improved delivery mechanisms to enhance retinal incorporation; a common problem in many proposed stem cell therapy systems. A multi-layered approach using both adipose and bone marrow derived stem cells, with appropriate microenvironment conditioning, may prove particularly effective at accomplishing protection and repair of the retinal microvascular network from diabetic damage.

Despite these substantial challenges, we contend our results suggest stem cell based strategies for diabetic retinopathy may allow a shift in focus from late destructive laser treatment of hypoxic retina in an attempt to save central vision, to earlier interventions aimed at stabilizing existing retinal microvasculature. Our present findings suggest this may be accomplished by hASCs through both direct contact of retinal microvessels as well as more general paracrine conditioning of the retinal microenvironment, both of which can prevent vessel loss and retinal hypoxia from occurring in the first place. The consistent and robust microvascular stabilizing properties of ASC derived pericytes offer hope that such a regenerative treatment for diabetic retinopathy may be attainable.

### Bibliography

### Example 2

1. ARVO. International Study Shows Global Prevalence of Diabetic Retinopathy. 2011; Available from: http://www.arvo.org/eweb/dynamicpage.aspx?WebCode=PressTemplate&wps_key=02 45bad8-3e36-4cf4-9b65-71e4693206fa.
2. Cogan, D. G., D. Toussaint, and T. Kuwabara, Retinal vascular patterns. IV. Diabetic retinopathy. Archives of Ophthalmology, 1961. 66: p. 366-78.
3. Romeo, G., et al., Activation of nuclear factor-kappaB induced by diabetes and high glucose regulates a proapoptotic program in retinal pericytes. Diabetes, 2002. 51(7): p. 2241-8.
4. Li, W., et al., Retinal capillary pericyte apoptosis in early human diabetic retinopathy. Chin Med J (Engl), 1997. 110(9): p. 659-63.
5. Kern, T. S., et al., Response of capillary cell death to aminoguanidine predicts the development of retinopathy: comparison of diabetes and galactosemia. Invest Ophthalmol Vis Sci, 2000. 41(12): p. 3972-8.
6. Jechlinger, M., et al., Autocrine PDGFR signaling promotes mammary cancer metastasis. J Clin Invest, 2006. 116(6): p. 1561-70.
7. Vincent, L. and S. Rafii, Vascular frontiers without borders: multifaceted roles of platelet-derived growth factor (PDGF) in supporting postnatal angiogenesis and lymphangiogenesis. Cancer Cell, 2004. 6(4): p. 307-9.
8. Chen, C. W., et al., Perivascular multi-lineage progenitor cells in human organs: regenerative units, cytokine sources or both? Cytokine Growth Factor Rev, 2009. 20(5-6): p. 429-34.
9. Armulik, A., et al., Pericytes regulate the blood-brain barrier. Nature, 2010. 468(7323): p. 557-61.
10. Hammes, H. P., et al., Pericytes and the pathogenesis of diabetic retinopathy. Diabetes, 2002. 51(10): p. 3107-12.
11. Armulik, A., G. Genove, and C. Betsholtz, Pericytes: developmental, physiological, and pathological perspectives, problems, and promises. Dev Cell, 2011. 21(2): p. 193-215.
12. Smith, L. E., et al., Oxygen-induced retinopathy in the mouse. Invest Ophthalmol Vis Sci, 1994. 35(1): p. 101-11.
13. Rakoczy, E. P., et al., Characterization of a mouse model of hyperglycemia and retinal neovascularization. Am J Pathol, 2010. 177(5): p. 2659-70.
14. Zuk, P. A., et al., Multilineage cells from human adipose tissue: implications for cell-based therapies. Tissue Eng, 2001. 7(2): p. 211-28.
15. Ding, L., et al., Endothelial and perivascular cells maintain haematopoietic stem cells. Nature, 2012. 481(7382): p. 457-62.
16. Katz, A. J., et al., Cell surface and transcriptional characterization of human adipose-derived adherent stromal (hADAS) cells. Stem Cells, 2005. 23(3): p. 412-23.
17. Hou, D., et al., Radiolabeled cell distribution after intramyocardial, intracoronary, and interstitial retrograde coronary venous delivery: implications for current clinical trials. Circulation, 2005. 112(9 Suppl): p. 1150-6.
18. Baba, T., et al., VEGF 165 b in the developing vasculatures of the fetal human eye. Dev Dyn, 2012. 241(3): p. 595-607.
19. Guillemin, G. J. and B. J. Brew, Microglia, macrophages, perivascular macrophages, and pericytes: a review of function and identification. J Leukoc Biol, 2004. 75(3): p. 388-97.
20. Hughes, S. and T. Chan-Ling, Characterization of smooth muscle cell and pericyte differentiation in the rat retina in vivo. Invest Ophthalmol Vis Sci, 2004. 45(8): p. 2795-806.
21. Armulik, A., A. Abramsson, and C. Betsholtz, Endothelial/pericyte interactions. Circ Res, 2005. 97(6): p. 512-23.
22. Cho, H., et al., Pericyte-specific expression of Rgs5: implications for PDGF and EDG receptor signaling during vascular maturation. FASEB J, 2003. 17(3): p. 440-2.
23. Pfister, F., et al., Pericyte migration: a novel mechanism of pericyte loss in experimental diabetic retinopathy. Diabetes, 2008. 57(9): p. 2495-502.
24. Walshe, T. E., et al., TGF-beta is required for vascular barrier function, endothelial survival and homeostasis of the adult microvasculature. PLoS One, 2009. 4(4): p. e5149.
25. Gaengel, K., et al., Endothelial-mural cell signaling in vascular development and angiogenesis. Arterioscler Thromb Vasc Biol, 2009. 29(5): p. 630-8.
26. Nowak, D. G., et al., Expression of pro- and anti-angiogenic isoforms of VEGF is differentially regulated by splicing and growth factors. J Cell Sci, 2008. 121(Pt 20): p. 3487-95.
27. Kutcher, M. E., et al., Pericyte Rho GTPase mediates both pericyte contractile phenotype and capillary endothelial growth state. Am J Pathol, 2007. 171(2): p. 693-701.
28. Otani, A., et al., Bone marrow-derived stem cells target retinal astrocytes and can promote or inhibit retinal angiogenesis. Nat Med, 2002. 8(9): p. 1004-10.
29. Ritter, M. R., et al., Myeloid progenitors differentiate into microglia and promote vascular repair in a model of ischemic retinopathy. J Clin Invest, 2006. 116(12): p. 3266-76.
30. Grant, M. B., et al., Adult hematopoietic stem cells provide functional hemangioblast activity during retinal neovascularization. Nat Med, 2002. 8(6): p. 607-12.
31. Sengupta, N., et al., The role of adult bone marrow-derived stem cells in choroidal neovascularization. Invest Ophthalmol Vis Sci, 2003. 44(11): p. 4908-13.
32. Dar-Oaknin, et al., 2012, U.S. Patent Application Publication 2012/0148546 A1, Pericyte Progenitor Cells and Methods of Generating and Using Same.
33. Katz and Cottler, 2008, U.S. Patent Application Publication 2008/0213235 (U.S. application Ser. No. 11/995,408), Adipose Tissue Stem Cells, Perivascular Cells, and Pericytes.
34. Amos, P J, et al., Functional binding of human adipose-derived stromal cells: effects of extraction method and hypoxia pretreatment. Ann Plast Surg. 2008 April; 60(4):437-44.
35. Amos, P J, et al., IFATS Series: The Role of Human Adipose-Derived Stromal Cells in Inflammatory Microvascular Remodeling and Evidence of a Perivascular Phenotype. Stem Cells. 2008 Apr. 24.
36. Traktuev, et al., Circ Res 102: 77-85, 2008.
37. Amos et al., 2011, Am. J. Physiol. Cell Physiol. 301:C1378-C1388 (first published Aug. 24, 2011).
38. Yang et al., 2010, Graefes Arch. Clin. Exp. Ophthalmol., Amelioration of diabetic retinopathy by engrafted human adipose-derived mesenchymal stem cells in streptozotocin diabetic rats.

### Example 3

### Introduction

The devastating consequences of traumatic open globe injuries are often seen well after the initial trauma, with loss of vision due to ongoing damage to the neural retina, uncontrolled aberrant neovascularization, and tractional retinal detachments from proliferative vitreoretinopathy (PVR). Between 2001 and 2006 in Operations Iraqi and Enduring Freedom 1086 incidents were reported of ocular trauma, including 198 open globe injuries requiring operative repair [5]. 75% of these injuries achieve a BCVA of 20/200 or worse [5]. Outside of the military setting there are an estimated 203,000 cases of open globe injury worldwide each year [6]. Even more worrisome is that on average 10% of the 700,000 closed globe rhegmatogenous retinal detachments that occur each year eventually develop PVR, with only 60% anatomic success rates for reattachment and even lower rates of functional vision recovery [7]. Unfortunately, there currently exist no therapies to prophylactically prevent these complications at the time of, or subsequent to, initial globe or detachment repair. These maladaptive responses of the eye to injury involve a complex coordination of proliferative cells in the retina including müller cells, infiltrating fibroblasts and lymphocytes, microglia, endothelial cells, and pericytes, designed to scar over the injury and re-perfuse tissues. The pathophysiology is highly suggestive of a retinal keloid, indicating a normal repair mechanism is being invoked that unfortunately goes awry, profoundly distorts the retinal architecture, and accelerates, rather than halts, vision loss [8].

Visual function could be better preserved through silencing or modulating this destructive process. Straightforward suppression of ocular inflammation through intra-ocular steroids has proven ineffective [9, 10]. A number of growth factors have been implicated in PVR including VEGF, SDF-1, PDGF, FGF, TNF-α, IL-6, and endothelin-1 all representing potential therapeutic targets [11-16]. The vitreous itself harbors factors that appear to promote trans-differentiation of RPE and other retinal cells into fibrotic cell types [17]. Although multiple potential therapeutics that can inhibit these factors have been suggested for disrupting fibrosis [14, 18-21], the efficacy of silencing any one factor seems unlikely to be the most productive approach in a multifactoral process involving multiple cell types and parallel paracrine signaling pathways. Rather, the goal for prevention of PVR should be to quickly restore the normal pre-injury ocular micro-environment, halting proliferative activity, and providing protection and trophic support to injured retinal cells.

We believe that restoration of normal retinal homeostasis can be best accomplished by using the multi-factoral anti-proliferative, anti-inflammatory, and neuro-protective secretome produced by administration of exogenous cultured perivascular mesenchymal stem cells (MSCs) [22, 23]. Recent work in other models of systemic disease and wound healing suggest a high level of cellular coordination in fibrotic scar formation [24]. Mesenchymal stem cells (MSCs) residing in the perivascular niche have been directly implicated as one of the key factors in this process contributing to both scar tissue formation through direct cellular transformation, as well as recruitment of inflammatory cell types to the injury site [25-27]. These cells, in concert with microglia in the central nervous system, serve as the sentinels for this injury process in host tissue and likely condition the micro-environment towards activation or suppression of the aberrant repair mechanisms that lead to PVR [28]. Along these lines, MSCs encapsulated in a gelatin-laminin scaffold are able to effectively promote neuronal survival and prevent glial scar formation in a brain injury model [29-31]. Factors produced by MSCs reduce tissue levels of INF-g, and their secretion of IL-4 promotes shifts in macrophages from a pro-inflammatory M1 to an anti-inflammatory M2 state [32]. In this capacity they appear to serve as the thermostat for tissue homeostasis tilting between tissue inflammation and quiescent tissue maintenance and regeneration as required, conditioning the microenvironment between these two states through multiple secretory factors. Exogenous introduction of MSC factors either from conditioned media or implanted cells themselves may also be able to modulate retina resident stem cell response given the known autocrine regulatory capabilities of these cells [33-37]. This makes these particular stem cells prime targets for potential therapeutics directed towards preventing at the top level PVR following retinal injury.

The therapeutic potential of embryonic and adult stem cells for retinal regeneration and repair has been evident for many years using numerous animal models of eye disease [38-40]. Rapid translation from bench to bedside, with embryonic, induced pluripotent stem cells (iPS), and allogeneic adult stem cell approaches (using bone marrow mesenchymal cells (BMSCs)) has led to a number of current Phase I clinical trials for the treatment of retinitis pigmentosa, Stargardt's disease, and macular degeneration. Most embryonic and iPS approaches rely on differentiation into a relevant cell type such as retinal pigment epithelial cells or photoreceptors prior to sub-retinal transplantation, with hoped for eventual functional integration into the retinal architecture [41, 42]. Adult stem cell approaches usually utilize undifferentiated stem cells based on the recognition that their general production of multiple trophic factors can help support damaged tissue, including retina, in injury models [43]. Autologous and allogeneic adult stem cells have clear clinical appeal given their ease of access, inherent pluripotency, and active suppression of immune responses, whereas iPS cells, may actually induce immunogenic responses in autologous recipients [44].

MSCs, specifically BMSCs, have demonstrated efficacy for treatment of murine models of choroidal neovascularization and proliferative retinopathy [45-51]. Human derived CD34+ BMSCs produce endothelial precursors that can assimilate into the endothelial layers of damaged retinal vasculature [52]. Intra-ocular human LIN(−) BMSCs accelerate vascular repair from a hyperoxic insult, via differentiation into microglial cell types [48]. In all instances the repair is predominantly paracrine in nature with sub-retinal transplantation of BMSCs able to prevent degeneration across the entire retina in an RCS rat model, and intra-vitreal injection of BMSCs able to prevent retinal ganglion cell loss in an optic nerve crush through secretion of neurotrophic factors [53, 54].

Unfortunately, BMSCs may worsen rather than prevent PVR, as they readily incorporate into laser-induced scar tissue via differentiation into fibrotic and endothelial cell types and are the predominant cell type in these membranes [55]. A more specific perivascular-derived MSC therapeutic approach for the prevention of retinal injury and PVR, now possible using adipose derived stem cells (ASCs), is directly in line with our understanding that these cells are a primary player in both priming and quelling the injury response in their host tissue [27]. Their widespread clinical utility is supported by their abilities to produce growth factors (e.g. GM-CSF, HGF, bFGF, and VEGF [56]) that favorably impact the repair of damaged tissues through the modulation of angiogenesis, inflammation, apoptosis, cell homing, cell proliferation, cell migration, and more.

ASCs have also been shown to play structural, “building block” roles in regenerating tissues by differentiating into a range of cell types in vivo, including myocyte, astrocyte, endothelial, and pericyte [57, 58]. Besides being the more relevant stem cell to modulate the retinal inflammatory microenvironment and thereby prevent PVR and promote tissue regeneration, ASCs are also more readily isolated and expanded quickly in larger numbers as compared to BMSCs making autologous therapy a possibility. All ongoing Phase I stem cells clinical trials currently require patients to be placed on lifelong immunosuppressants to prevent rejection. The systemic risk as opposed to ocular benefit of these trials remains to be clarified. Encapsulated ASC therapy would not require immunosuppression.

Adult human adipose-derived stem cells (hASCs) will naturally differentiate in culture into pericytes. Injected intra-vitreally these ASCs can integrate with and stabilize the retinal microvasculature, prevent cellular loss, and inhibit scar formation in response to retinal injury [2-4, 55]. More specifically, these perivascular ASCs prevent capillary dropout in an inflammatory micro-environment in vivo and directly inhibit endothelial cell proliferation in vitro; a key step in scar tissue formation (Example 3, FIGS. 2 and 3). Intra-vitreal ASCs down regulate key inflammatory factors such as the chemokine ccl2 and endothelin-2, and GFAP expression on Mueller cells is markedly diminished [3]. They protect against endogenous pericyte and endothelial cell loss, as well as maintain retinal function as measured by ERG [3]. The paracrine aspect of these protective effects is most clearly demonstrated by recent studies that show conditioned media from these cells themselves when injected intravitreally can prevent retinal damage from phototoxic injury via secreted progranulin as well as other trophic factors [2]. Taken together these studies all suggest that introduction of cultured autologous ASC-derived pericytes, or even conditioned media produced by these cells, can potentially revert the traumatic ocular micro-environment back to its pre-injury state and thereby restore retinal homeostasis and promote cell survival.

The ultimate goal of these pre-clinical studies is to advance use of ASCs for the prevention of PVR and trophic support of the retina into eventual Phase I human clinical trials. However, doing so requires answering a number of outstanding questions that are the focus of the present proposal. While it is certainly feasible to perform intravitreal injections of isolated ASCs, we believe there are more effective, reproducible, and established approaches that will enhance treatment efficacy and longevity of any treatment effect, making a positive outcome more likely. An encapsulated cell implant placed in the vitreous space has been shown to effectively provide trophic factors such as CNTF to the retina using genetically engineered cells, with positive effects on retinal disease [59, 60]. Viability of the encapsulated cells is maintained up to 18 months post-implantation. In pre-clinical models BMSCs have been successfully encapsulated in alginate based MicroBeads and delivered sub-retinally, with viability as indicated by maintenance of GFP reporter gene expression from these cells for at least 120 days [61]. Encapsulation would permit the use of allogeneic ASCs without immunosuppression, allowing for an implant to be prepared and placed at the time of open-globe or retinal detachment repair. It would also eliminate the need for ASC isolation from the patient themselves. Given the acute timecourse under which PVR develops following trauma or retinal detachment, short-term use of encapsulated cell technology would be the preferred approach.

We believe these data establish a strong case for the central involvement of perivascular stem cells in the development of the retinal fibrosis that is the cardinal feature of PVR. Intravitreally injected ASCs have the key ability to regulate the pro-inflammatory retinal microenvironment that results from retinal injury, reducing levels of key cytokines and molecular players implicated in the development of PVR. They can ameliorate production of glial scar tissue in response to brain injury. The path toward Phase I clinical trials for PVR is well defined, with no insurmountable obstacles to be overcome in developing an encapsulated cell implant for use in patients.

Traumatic open globe injuries continue to be one of the most vexing problems for retina specialists. A vicious cascade of proliferative scar tissue follows after seemingly successful repair of the injured eye, which leads to permanent retinal detachment and ultimately blindness. There are no available therapies and anti-inflammatory approaches, such as intra-ocular steroids, fail to ameliorate this process. Proliferative vitreoretinopathy (PVR) represents a normal repair process gone awry leading to a reactive gliosis and fibrosis of the end organ, be it the eye, brain, or kidney. An unlikely cell, the pericyte, has emerged as the key player that orchestrates this fibrosis. These multi-potent perivascular mesenchymal stem cells (MSCs) are activated in injury and directly regulate inflammation, proliferation, and recruitment of intra-ocular and extra-ocular scar forming cells to the site of injury, predominantly through paracrine means. Specific function of these MSCs is driven by the needs of the host tissue, altering their secretome to tamp down inflammation and enforce tissue homeostasis during normal function or ramp up cell activation and proliferation during retinal injury to effect tissue repair. (See Example 3, FIG. 1—Example 3, FIG. 9).

Without wishing to be bound by any particular theory, it is proposed herein to harness the secretome of these MSCs to condition the ocular micro-environment back to its pre-injury state and thereby prevent formation of scar tissue. This can be accomplished by implanting encapsulated perivascular stem cells that are biased towards their normal quiescent anti-proliferative, anti-inflammatory, and neuro-protective state. A quiescent state can be enforced by use of small molecules embedded within the encapsulant, such as TGF-beta-1, ensuring homeostasis. Toward this end, we will use pericytes differentiated from human adipose derived stem cells (hASCs), which we, and others, have previously demonstrated can inhibit inflammation, prevent retinal cell death, and prevent scar formation during retinal tissue repair[1-4]. We believe the complex conditioned secretome of these quiescent MSCs will directly inhibit multiple signaling pathways that initiate PVR and promote survival of injured retinal cells, providing a more effective approach than therapeutics that propose to inhibit individual pro-inflammatory or pro-proliferative factors.

### Research Strategy

### Example 3

1: Determine the Most Feasible Encapsulated Cell Technology to Use with hASCs and Demonstrate its Efficacy Using In Vitro Model Systems of Retinal Injury and Proliferative Vitreoretinopathy

Task 1.1: Establish Baseline Characteristics of Non-Encapsulated hASCs Through Multiplex Analysis

Rationale: Non-encapsulated hASCs can regulate the pro-PVR micro-environment through paracrine secretion of trophic and regulatory factors [3]. Establishing baseline characteristics of this secretome provides a point of comparison for encapsulated hASCs and a potential marker panel for ensuring cellular competency. Design: 1) Fat will be harvested from a human donor and the stromal vascular fraction plated in culture per established protocols [4]; 2) 24 hour conditioned media from passage 4 stem cells will be run for multiplex analysis via a Luminex 100 IS system to examine the secretome for angiogenic, inflammatory, neuroprotective and proliferative factors previously identified as involved in the development of PVR [11, 17, 62].

Expected Results: We expect elevated levels of previously identified mesenchymal stem cell secreted factors such as IGF-1, HGF, VEGF, TGF-β, BDNF, FGF-2, GDNF, NGF, and SCF and can derive a relative expression profile of these factors as a marker to predict stem viability and efficacy.

Alternative Approach: Trials of multiple multiplex assays and cultured cells from different donors may be required to determine a reproducible fingerprint predictive of cellular efficacy in preventing PVR.

Task 1.2: Add Reporter GFP Construct to Cultured hASCs

Rationale: A GFP reporter introduced into hASCs can provide a surrogate marker for cell viability in vivo [3].

Design: 1) Cultured passage 4 hASCs will be labeled with lentiviral GFP per standard procedures [3]; and 2) After introduction of the vector, cells expressing high levels of GFP will be selected using FACS sorting for further clonal expansion and encapsulation.

Expected Results: A stable expression of GFP in at least 20% of cultured cells can be established with stable expression following clonal expansion of these cells. Alternative Approach: Transfection with a GFP plasmid can be considered if lentiviral introduction of GFP is suboptimal as measured by number of cells or levels of expression [61]. The lipophilic fluorescent marker DiI can also be used as a secondary method to label and track hASCs [4].

Task 1.3 Assess Encapsulation Technology Effects on hASC Viability and Secretome Via Multiplex Analysis

Rationale: Encapsulation of hASCs would allow for establishment of a stable conditioned micro-environment and easier implantation into human eye. Allogeneic cells could be used without concern over immunogenicity. However, encapsulation could potentially alter cellular viability and secretome in unexpected ways.

Design: 1) GFP labeled hASCs will be introduced into CellBead and MicroBead alginate matrix [61]; 2) Encapsulated cells will be placed in serum free culture for 24 hours and media harvested for comparative multiplex analysis using Task 1.1 as an established baseline; and 3) Cell viability will be assessed in long-term culture of CellBeads and MicroBeads through monitoring of GFP signal in individual cells, use of a Live/Dead Viability/Cytotoxicity Kit, and multiple time point multiplex analysis of conditioned media over 6 months [61, 63].

Expected Results: Cells will remain viable for a minimum of 4 months and maintain a comparable secretome to non-encapsulated cells during that time.

Alternative Approach: CellBeads have been chosen due to their GMP-ready status and successful prior use with hMSCs [61, 64, 65]. If viability is poor or secretome is inexorably altered, substitute encapsulation technologies can be considered, including a collagen-alginate composite[66] or an ECT like platform as Neurotech uses already in clinical trials [59, 60]. Size limitations may dictate choice of animal model (rat, rabbit) or selection of implant. Cell survival may be enhanced by immortalization of the hASC cell line using a human telomerase reverse transcriptase gene [61, 67].

### Task 1.4/1.5 Evaluate Encapsulated Cell Effects on Cellular Proliferation and Migration of ARPE, Endothelial, and Retinal Pericytes in Culture

Rationale: Formation of PVR requires the coordinated migration and proliferation of multiple cell types including RPE cells, microglia, endothelial cells, and pericytes. Secreted factors in quiescent hASCs are known to prevent endothelial and pericyte proliferation and prevent apoptosis of retinal cells [3, 4, 68]. There is a need to evaluate whether these effects are maintained by encapsulated hASCs as well as establish potential interactions with RPE and microglial cells.

Design: 1) Cellular proliferation of separate low density RPEJ, HUVEC, microglia, and bovine retinal pericyte cultures will be determined both with and without the presence of encapsulated hASCs; 2) Ability for encapsulated hASCs to inhibit cellular migration will be determined using a Transwell assay both under normal conditions as well as in the presence of VEGF165 stimulation[63]; 3) Apoptosis of low density cultured RPEJ, HUVEC, and bovine retinal pericytes in the presence or absence of encapsulated hASCs will be assessed using standard apoptosis detection kits[3, 63]; and 4) Relative effectiveness of conditioned media from encapsulated cells versus the encapsulated hASCs themselves will be determined for select conditions and cell types.

Expected Results: 1) Secreted factors from encapsulated hASCs will inhibit cellular proliferation, migration, and apoptosis of RPEJ, HUVEC, microglia, and retinal pericytes; and 2) Conditioned media will have a more pronounced effect at early time points, but show equivalence with use of encapsulated hASCs themselves at later time points, with a clear dose response curve.

Alternative Approach: 1) Pre-conditioning of culture media with encapsulated hASCs may be required prior to low density cell plating to improve measured efficacy of treatment effects [4]; and 2) Ability to inhibit cell proliferation and migration does not necessarily imply a treatment effect in in vivo models and thus will not preclude completing Aim 2 in parallel.

Task 1.6/1.7 Determine Ability of Encapsulated hASCs to Maintain Baseline Secretome in Presence of an Inflammatory and Hypoxic Stimulus.

Rationale: Environmental conditions, such as relative hypoxia of the vitreous or presence of inflammatory cell types in the eye, have the potential to alter the secretome of encapsulated hASCs and thereby effect therapeutic efficacy [69]. Pre-conditioning of the hASCs themselves or the alginate implant with small molecules, or perhaps the entire ocular microenvironment with use of pre-conditioned media co-injected at the time of implant may better maintain baseline hASC secretome.

Design: 1) Encapsulated hASCs will be cultured by themselves in normoxia, by themselves in hypoxia, and co-cultured with CD14+ human monocytes per established protocol [69]; 2) Multiplex analysis of culture supernatants will be performed per Task 1.1.; 3) RNA will be isolated from encapsulated hASCs in all conditions to examine changes in gene expression due to micro-environmental changes and paracrine interactions with CD14+ monocyte derived macrophages[69]; 4) Encapsulated hASCs preconditioned with TGF-beta1/other small molecule prior to encapsulation and alginate gel embedded with TGF-beta1/other small molecule will be compared against unmodified encapsulated hASCs to determine if secretome and gene expression of hASCs is better stabilized by pre-conditioning; 5) Conditioned media from encapsulated hASCs will be added to hypoxia and macrophage co-culture conditions to see if secretome and gene expression of hASCs is better stabilized by use of conditioned media at the time of implantation.

Expected Results: 1) Relative hypoxia or co-culture with macrophages will likely cause secretion of more pro-angiogenic and pro-inflammatory molecules than under baseline conditions including VEGF, IL-1beta, MIP-1alpha, and IFN-gamma; 2) This effect can be ameliorated by pre-conditioning hASCs or their alginate encapsulant with small molecules at the time of co-culture; and 3) normoxic hASC conditioned media can be administered at the time of co-culture to lower the relative secretion of pro-inflammatory cytokines.

Alternative Approach: 1) If encapsulated hASC secretome is inexorably altered toward a pro-inflammatory phenotype as revealed by hypoxia, sub-retinal versus intra-vitreal implantation may be the preferred route for experiments of Aim 2 to maintain more normoxic conditions; 2) If conditioned media better stabilizes the secretome of hASCs in either hypoxia or co-culture conditions, it would suggest administration of normoxic hASC conditioned media at the time of implant may improve therapeutic efficacy; and 3) If use of normoxic hASC conditioned media itself is able to inhibit pro-inflammatory secretions from macrophages, then a focus more on intermittent intra-ocular injection with conditioned media in the setting of ocular injury may be warranted.

2: Determine Ability of Sub-Retinal/Intra-Vitreal Encapsulated hASCs to Prevent PVR.

Task 2.1: Assess Viability and Biocompatibility of Encapsulated hASCs in Wildtype Mice Following Sub-Retinal or Intra-Vitreal Implantation.

Rationale: Sub-retinal implantation of encapsulated hASCs may enable greater viability that intra-vitreal injection, with the need for acute versus long-term treatment guiding approach.

Design: 1) Up to 10 MicroBeads encapsulating hASCs will be implanted into either the vitreous or choroidal space of 6 week old C57/b16 mice, with the contralateral eye serving as the control (n=10 eyes of 10 mice each condition); 2) GFP expression will be used to assess cell viability up to 6 months using a Heidelberg Specralis retinal imager with OCT used to assess retinal microstructure over or underlying each bead; 3) At 1 week, 2 weeks, 1 month, 2 months, and 4 month time points, implanted eyes will be harvested, implanted beads retrieved, and retinas collected; 4) Retinal lysates will be collected for multiplex analysis per Task 1.1 to assess for induction of ocular inflammation; 5) A Live/Dead Kit will be used on some explanted MicroBeads to assess hASC viability at each time-point as compared to GFP imaging; and 6) Some explanted MicroBeads from each time-point and condition will be cultured for 24 hours and secretome of conditioned media analyzed per multiplex analysis of Task 1.1

Expected Results: 1) We expect encapsulated hASCs to be well tolerated with no induction of inflammatory markers in retinal lysates; 2) Up to 120 days of continuous GFP expression will be found for implanted MicroBeads as per prior studies [61]; 3) Cell viability will be enhanced for sub-retinal compared intra-vitreal implantation; 4) GFP analysis will be consistent with Live/Dead kit analysis, allowing GFP to serve as a surrogate marker for cell viability; and 5) Relative secretome will be maintained during the 4 month time-course but with progressively reduced levels owing to loss of hASCs in the encapsulant.

Alternative Approach: 1) If use of larger CellBeads is required, we may switch to a Sprague Dawley wildtype rat for viability and biocompatibility studies; 2) Use of mice with genetically tagged fluorescent microglia may offer the ability to better assess retinal inflammation than wildtype mice; and 3) If not linearly correlated, a relative scale between cell viability as assessed by GFP and Live/Dead analysis will be determined for subsequent murine retinal injury models

Task 2.2/2.4: Assess Ability of Encapsulated hASCs or their Conditioned Media to Ameliorate Laser Induced Scar Injury

Rationale: Laser injury to the mouse retina induces brisk inflammation with formation of sub-retinal scar tissue that can serve as a pre-clinical model for acute proliferative vitreoretinopathy. A reset of the ocular micro-environment to its baseline pre-injury state by use of encapsulated hASCs alone or in concert with hASC-conditioned media produced by these cells may impair the inflammatory cascade and cellular migration that contributes to scar formation.

Design: 1) Sub-retinal scar tissue will be produced by laser photocoagulation (75-μm spot size, 0.1-second duration, 120 mW) induced rupture of Bruch's membrane (n=20 eyes of 20 mice, 4 laser spots equidistant from optic nerve per eye) in 6 week old C57bl/6 wildtype mice; 2) In 10 of these mice sub-retinal implantation of MicroBeads will be performed immediately following laser injury and in another 10 mice pars-plana injection of 1 ul concentrated hASC conditioned media will be performed; 3) 1 week following injury fluorescein angiography will be performed via intraperitoneal injection of fluorescein [4] to determine extent of scar tissue formation via extravasation of dye; 4) Eyes will be harvested and bio-microscopy performed on whole mount retina to confirm the fluorescein findings; 5) Quantification of total scar size for control laser burn only, implanted hASCs alone, hASC conditioned media alone, and implanted hASCs with hASC conditioned media will be compared 5) Lectin whole mount labeling of the retinal vasculature along with antibodies to endothelial cells, microglia, macrophages, RPE, and pericytes will be used to quantify cell type incorporation into the resultant scar per established protocols[4, 55]. 6) Multiplex analysis of retinal lysates will be performed to examine hASC effects on pro-angiogenic and pro-inflammatory pathways per Task 1.1

Expected outcomes: 1) Laser induced scar formation at day 7 in control eyes will be larger that the other three hASC treated conditions; 2) Lower total numbers of immunolabeled microglia, pericytes, and RPE will be seen in the resultant scar, consistent with the smaller scar size; and 3) Pro-inflammatory and pro-angiogenic factors will be diminished in hASC treated retinae compared to control eyes.

Alternative Approach: 1) It is possible that implanted hASCs or their conditioned media will have no discernible effect on scar formation due to the significant bone marrow contribution to these scars following laser injury [55, 70]. However, this outcome seems less likely given that multiple intraocular measures to reduce the inflammatory micro-environment following injury such as inhibition or microglia, VEGF signaling, or cell migration can substantially reduce scar formation [71-73]. 2) hASC conditioned media may be more efficacious than an encapsulated hASC implant due to the acute aspect of the injury, indicating that injection of hASC conditioned media may be the preferred route for acute treatment of ocular injury, with the implant serving longer term suppression of pro-inflammatory signals and providing trophic factors to surviving retinal cells.

Task 2.3/2.5 Compare Efficacy of Encapsulated hASCs or their Conditioned Media to Inhibit Formation of Proliferative Vitreoretinopathy

Rationale: Intravitreal injection of dispase, a protease that cleaves collagen IV, is an established model of PVR in mice [74]. Pre-clinical studies are needed in a relevant murine model of pre-retinal PVR to determine whether encapsulated hASCs or their conditioned media can inhibit the formation of scar tissue. Quiescent hASCs or their conditioned media should reset the inflammatory ocular micro-environment created by dispase injection such that pre-retinal membrane formation is substantially reduced.

Design: 1) 3 μl of dispase at a concentration of 0.2 U/μl will be injected into the vitreal cavities of 6 week old C57bl/6 wildtype mice (n=120 eyes of 120 mice) using a Hamilton syringe, with saline injected in an addition 24 control mice; 2) 1 day post dispase treatment a subset of mice will receive intra-vitreal implantation of encapsulated hASC MicroBeads, intra-vitreal implantation of MicroBeads plus hASC conditioned media, hASC conditioned media alone, or equal volume of unconditioned media (24 eyes of 24 mice each group); 3) At 8 weeks mesopic and scotopic ERG will be performed on treated mice (4 per each treatment group); 4) At 1 day, 3 days, 1 week, 2 weeks, 4 weeks and 8 weeks post injection eyes will be harvested (4 mice per each treatment group) and whole mount biomicroscopy will be performed along with immunohistochemistry for pericytes, microglia, endothelial cells, macrophages, and RPE cells; 5) Retinal lysates will be prepared for multiplex analysis as per Task 1.1 (2 mice per each treatment group and timepoint)

Expected outcomes: 1) Early inflammatory biomarkers that have been demonstrated for this model will be downregulated in all hASC treatment groups as compared to dispase alone controls 2) Decreased early infiltrating neutrophils will be seen in hASC treatment groups at early time points 3) ERG will be preserved at late time points in hASC treated mice compared to dispase only controls 4) Decreased incorporation of microglia, RPE, Muller, endothelial cells, and pericytes into PVR membranes as compared to controls.

Alternative Approach: 1) Encapsulated hASCs or their conditioned media may be insufficient to prevent PVR formation in this murine model. If so, a number of hASC pre-conditioning strategies will be attempted to improve efficacy of these cells through small molecules such as TGF-β1 either prior to encapsulation or within the alginate gel itself. 2) If pre-conditioning does not work, inhibition of individual pro-inflammatory or pro-angiogenic factors determined to be upregulated in the retinal lysate, i.e., SDF-1, VEGF or PDGF-alpha, will be performed in concert with hASC implantation to determine if additional efficacy is afforded with hASC over single factor inhibition. 3) Secretome and proliferative activity of encapsulated cells will be examine and if found substantially altered in this model, we will look for factors or encapsulation conditions that can better quench the activation of our implanted cells.

3: Derive a GMP Level hASC Encapsulated Cell Implant and Evaluate Retinal Protection in Murine Models of Retinal Injury.

Task 3.1: Arrange Pre-IND Meeting with FDA to Establish Data Required for Potential Phase I Trial.

Rationale: Producing data sufficient for Phase I IND application requires consultation with the FDA with respect to their published guidance documents and specifics issues that pertain to our proposed therapeutic. Is-sues to be addressed include acceptable safety and efficacy measures as well as certification process required for validating product efficacy.

Design: Our group has already reached out to the FDA ophthalmics division seeking to establish a guidance pathway for proceeding with GMP harvest of cultured ASCs and validation of cell efficacy. We will advance this discussion as the form and substance of the proposed implantable therapeutic advances based on results from Aim 1 and 2.

Expected Results: None

Alternative Approach: None

Task 3.2/3.3 Derive GMP Quality hASCs that do not Require Fetal Bovine Serum as Part of their Growth Media and Demonstrate Comparable Proliferation and Secretome.

Rationale: To move forward with Phase I human clinical trials, GMP grade ASCs are required. We will again use multiple established murine models to recapitulate the predominate characteristics of PVR.

Design: 1) Establish SOP for isolation and harvest of fat from donor; 2) Obtain fat from donor and isolate under GMP conditions in GMP facility; 3) Culture isolated hASCs in serum free media, comparing different formulations available from commercial vendors, including DMEM, with respect to cell proliferation. Establish parallel cultures of hASCs in usual fetal bovine serum (FBS) conditions for comparison; 4) Compare secretome via multiplex assay of serum-free and FBS raised hASCs by conditioning media for 24 hours, to determine impact of serum-free conditions on hASC function per Task 1.1; and 5) Optimize serum-free media composition to produce comparable secretome and proliferation to FBS conditions.

Expected Results: 1) hASC product in serum free conditions that demonstrates comparable growth and secretome to hASCs cultured with FBS

Alternative Approach: 1) If hASC secretome is altered, additional in vivo validation of potential rescue of PVR models is required to determine impact on therapeutic efficacy; and 2) Consider use of human serum, or individual growth factor supplementation to serum free conditions to replace FBS bioactivity if no non-serum approach proves viable.

### Task 3.4 Establish a Quality Control Process Using Multiplex Analysis and In Vitro Assays of Activity.

Rationale: To move forward with Phase I human clinical trials, a method for validating quality and composition of GMP grade ASCs is required. In vitro assays as pioneered in Aim 1 can be used to establish a minimum threshold secretory signature and bioactivity prior to use of encapsulated cells in patients.

Design: 1) Establish SOP for encapsulation process using GMP quality CellBeads; 2) Compare secretome as per Task 1.1 of different donor GMP hASC isolates to determine the most reliable and repeatable factors that can be used to assess hASC competency; 3) Establish parameters for migratory assay per Task 1.4/1.5 that can be used to assay for bioactivity of CellBeads on target RPE or pericyte cell lines; and 4) Create SOP for verifying acceptable CellBead secretome and bioactivity.

Expected Results: 1) Levels of key secreted factors such as IGF and VEGF will serve to validate encapsulated cell competency for use in clinical trials; and 2) A reliable bioassay of target cell migration and proliferation can be established with validated minimum and maximum limits.

Alternative Approach: 1) Complete lack of donor to donor consistency is not anticipated based on our results with multiplex analysis as well as the results of others. However, it would be possible to establish an alternate in vivo assay for implant bioactivity using the murine PVR model.

Task 3.5/3.6 Compare Efficacy of GMP hASC Implant and Implant Conditioned Media to Inhibit PVR and Retain Retinal Function, and Identify any Implant Safety Issues Regarding Immunogenicity/Tumorigenicity

Rationale: Intravitreal injection of dispase, as per Task 2.3 is the most direct pre-clinical model for evaluating efficacy of GMP CellBead encapsulated hASCs to inhibit

PVR. Moving forward towards Phase 1 clinical trials requires justification by demonstrating implant efficacy to treat PVR, retained retinal function, and verification of implant safety.

Design: 1) 3 μl of dispase at a concentration of 0.2 U/μl will be injected into the vitreal cavities of 6 week old C57bl/6 wildtype mice (n=40 eyes of 40 mice) as per Task 2.3. 2) 1 day post dispase treatment a subset of mice will receive intra-vitreal implantation of GMP encapsulated hASC CellBeads plus hASC conditioned media, or equal volume of unconditioned media (20 eyes of 20 mice per group). 3) At 8 weeks mesopic and scotopic ERG will be performed on treated mice (4 per each treatment group) 4) At 1 day, 1 week, 4 weeks and 8 weeks post injection eyes will be harvested (4 mice per each treatment group) and whole mount biomicroscopy will be performed along with immunohistochemistry for cell types incorporated into the scar per Task 2.3. 5) Retinal lysates will be prepared for multiplex analysis as per Task 1.1 (2 mice per each treatment group and timepoint) 6) Encapsulated cells will be explanted and examined for cell viability and maintenance of secretome as per Task 2.1, as well as differentiation into alternate cell types including adipocyte, chondrocyte, or osteocyte) Harvest of lung, spleen, liver, and kidney will be performed to examine for hASC tracking outside the eye and/or tumorigenicity.

Expected outcomes: 1) Pro-inflammatory biomarkers will be downregulated in all hASC treatment groups as compared to dispase alone controls; 2) Decreased early infiltrating neutrophils will be seen in hASC treatment groups at early time points; 3) ERG will be preserved at late time points in hASC treated mice compared to dispase only controls; 4) Decreased incorporation of microglia, RPE, Muller, endothelial cells, and pericytes into PVR membranes as compared to controls; 5) No end-organ trafficking of hASC nor tumor formation will be observed; and 6) Secretome of encapsulated hASCs will be maintained out to 8 weeks post implantation

Alternative Approach: As per Task 2.3

### Statistics:

We have calculated the study group sizes in Aim 2 and 3 based on a Power Analysis performed using our preliminary data with alpha level=0.05 and P=0.8. To obtain approximate total mice numbers shown above, we have multiplied the number of mice per group by the number of treatment groups and by the injection/harvest time points, while considering the number of mice needed for each of the relevant assays within the different experiments.

### Bibliography

### Example 3

1. Zhu, W., et al., Expression of IGFBP6 in proliferative vitreoretinopathy rat models and its effects on retinal pigment epithelial J cells. Mol Med Rep, 2014. 9(1): p. 33-8.
2. Tsuruma, K., et al., Progranulin, a major secreted protein of mouse adipose-derived stem cells, inhibits light-induced retinal degeneration. Stem Cells Transl Med, 2014. 3(1): p. 42-53.
3. Rajashekhar, G., et al., Regenerative therapeutic potential of adipose stromal cells in early stage diabetic retinopathy. PLoS One, 2014. 9(1): p. e84671.
4. Mendel, T. A., et al., Pericytes derived from adipose-derived stem cells protect against retinal vasculopathy. PLoS One, 2013. 8(5): p. e65691.
5. Weichel, E. D., et al., Combat ocular trauma visual outcomes during operations iraqi and enduring freedom. Ophthalmology, 2008. 115(12): p. 2235-45.
6. Schmidt, G. W., et al., Vision survival after open globe injury predicted by classification and regression tree analysis. Ophthalmology, 2008. 115(1): p. 202-9.
7. Pastor, J. C., Proliferative vitreoretinopathy: an overview. Surv Ophthalmol, 1998. 43(1): p. 3-18.
8. Morescalchi, F., et al., Proliferative vitreoretinopathy after eye injuries: an overexpression of growth factors and cytokines leading to a retinal keloid. Mediators Inflamm, 2013. 2013: p. 269787.
9. Ahmadieh, H., et al., Triamcinolone acetonide in silicone-filled eyes as adjunctive treatment for proliferative vitreoretinopathy: a randomized clinical trial. Ophthalmology, 2008. 115(11): p. 1938-43.
10. Yamakiri, K., et al., One-year results of a multicenter controlled clinical trial of triamcinolone in pars plana vitrectomy. Graefes Arch Clin Exp Ophthalmol, 2008. 246(7): p. 959-66.
11. Banerjee, S., et al., Multiplex bead analysis of vitreous humor of patients with vitreoretinal disorders. Invest Ophthalmol Vis Sci, 2007. 48(5): p. 2203-7.
12. Cui, J., et al., PDGF receptors are activated in human epiretinal membranes. Exp Eye Res, 2009. 88(3): p. 438-44.
13. Harada, C., Y. Mitamura, and T. Harada, The role of cytokines and trophic factors in epiretinal membranes: involvement of signal transduction in glial cells. Prog Retin Eye Res, 2006. 25(2): p. 149-64.
14. Iribarne, M., et al., Blockade of endothelinergic receptors prevents development of proliferative vitreoretinopathy in mice. Am J Pathol, 2008. 172(4): p. 1030-42.
15. Lei, H. and A. Kazlauskas, Growth factors outside of the platelet-derived growth factor (PDGF) family employ reactive oxygen species/Src family kinases to activate PDGF receptor alpha and thereby promote proliferation and survival of cells. J Biol Chem, 2009. 284(10): p. 6329-36.
16. Roldan-Pallares, M., et al., Preoperative duration of retinal detachment and subretinal immunoreactive endothelin-1: repercussion on logarithmic visual acuity. Graefes Arch Clin Exp Ophthalmol, 2010. 248(1): p. 21-30.
17. Wladis, E. J., et al., Analysis of the molecular biologic milieu of the vitreous in proliferative vitreoretinopathy. Retina, 2013. 33(4): p. 807-11.
18. Kawahara, S., et al., Potent inhibition of cicatricial contraction in proliferative vitreoretinal diseases by statins. Diabetes, 2008. 57(10): p. 2784-93.
19. Lei, H., M. A. Rheaume, and A. Kazlauskas, Recent developments in our understanding of how platelet-derived growth factor (PDGF) and its receptors contribute to proliferative vitreoretinopathy. Exp Eye Res, 2010. 90(3): p. 376-81.
20. Nassar, K., et al., The novel use of decorin in prevention of the development of proliferative vitreoretinopathy (PVR). Graefes Arch Clin Exp Ophthalmol, 2011. 249(11): p. 1649-60.
21. Turgut, B., et al., The impact of tacrolimus on growth factors in experimental proliferative vitreoretinopathy. Retina, 2012. 32(2): p. 232-41.
22. Gao, L., et al., The immunosuppressive properties of non-cultured dermal-derived mesenchymal stromal cells and the control of graft-versus-host disease. Biomaterials, 2014.
23. Murphy, M. B., K. Moncivais, and A. I. Caplan, Mesenchymal stem cells: environmentally responsive therapeutics for regenerative medicine. Exp Mol Med, 2013. 45: p. e54.
24. Campanholle, G., et al., Cellular mechanisms of tissue fibrosis. 3. Novel mechanisms of kidney fibrosis. Am J Physiol Cell Physiol, 2013. 304(7): p. C591-603.
25. Khairoun, M., et al., Renal ischemia-reperfusion induces a dysbalance of angiopoietins, accompanied by proliferation of pericytes and fibrosis. Am J Physiol Renal Physiol, 2013. 305(6): p. F901-10.
26. Pan, S. Y., Y. T. Chang, and S. L. Lin, Microvascular pericytes in healthy and diseased kidneys. Int J Nephrol Renovasc Dis, 2014. 7: p. 39-48.
27. Ren, S. and J. S. Duffield, Pericytes in kidney fibrosis. Curr Opin Nephrol Hypertens, 2013. 22(4): p. 471-80.
28. Soderblom, C., et al., Perivascular fibroblasts form the fibrotic scar after contusive spinal cord injury. J Neurosci, 2013. 33(34): p. 13882-7.
29. Sarnowska, A., et al., Encapsulation of mesenchymal stem cells by bioscaffolds protects cell survival and attenuates neuroinflammatory reaction in injured brain tissue after transplantation. Cell Transplant, 2013. 22 Suppl 1: p. S67-82.
30. Crigler, L., et al., Human mesenchymal stem cell subpopulations express a variety of neuro-regulatory molecules and promote neuronal cell survival and neuritogenesis. Exp Neurol, 2006. 198(1): p. 54-64.
31. Teixeira, F. G., et al., Mesenchymal stem cells secretome: a new paradigm for central nervous system regeneration? Cell Mol Life Sci, 2013. 70(20): p. 3871-82.
32. Choi, E. W., et al., Transplantation of CTLA4Ig gene-transduced adipose tissue-derived mesenchymal stem cells reduces inflammatory immune response and improves Th1/Th2 balance in experimental autoimmune thyroiditis. J Gene Med, 2011. 13(1): p. 3-16.
33. Katsura, M. K., et al., Growth regulation of bovine retinal pericytes by transforming growth factor-beta2 and plasmin. Curr Eye Res, 2000. 20(3): p. 166-72.
34. Scheef, E. A., C. M. Sorenson, and N. Sheibani, Attenuation of proliferation and migration of retinal pericytes in the absence of thrombospondin-1. Am J Physiol Cell Physiol, 2009. 296(4): p. C724-34.
35. Wu, P., et al., Hypoxia down-regulates endostatin production by human microvascular endothelial cells and pericytes. Biochem Biophys Res Commun, 2001. 288(5): p. 1149-54.
36. Yamagishi, S., et al., Pigment epithelium-derived factor (PEDF) promotes growth of pericytes through autocrine production of platelet-derived growth factor-B. Microvasc Res, 2005. 69(3): p. 128-34.
37. Yamagishi, S., et al., Vascular endothelial growth factor acts as a pericyte mitogen under hypoxic conditions. Lab Invest, 1999. 79(4): p. 501-9.
38. Dahlmann-Noor, A., et al., Current approaches and future prospects for stem cell rescue and regeneration of the retina and optic nerve. Can J Ophthalmol. 45(4): p. 333-41.
39. Jin, Z. B., et al., Induced pluripotent stem cells for retinal degenerative diseases: a new perspective on the challenges. J Genet, 2009. 88(4): p. 417-24.
40. Lamba, D. A., M. O. Karl, and T. A. Reh, Strategies for retinal repair: cell replacement and regeneration. Prog Brain Res, 2009. 175: p. 23-31.
41. James Bainbridge, B. D., A Phase I/II, Open-Label, Multi-Center, Prospective Study to Determine the Safety and Tolerability of Sub-retinal Transplantation of Human Embryonic Stem Cell Derived Retinal Pigmented Epithelial (hESC-RPE) Cells in Patients With Stargardt's Macular Dystrophy (SMD). Feb. 1, 2013: Clincal Trials Gov.
42. Schwartz, S. D., et al., Embryonic stem cell trials for macular degeneration: a preliminary report. The Lancet, 2012. 379(9817): p. 713-720.
43. Siqueira, R. C., et al., Intravitreal injection of autologous bone marrow-derived mononuclear cells for hereditary retinal dystrophy: a phase I trial. Retina, 2011. 31(6): p. 1207-14.
44. Zhao, T., et al., Immunogenicity of induced pluripotent stem cells. Nature, 2011. 474(7350): p. 212-5.
45. Banin, E., et al., T2-TrpRS inhibits preretinal neovascularization and enhances physiological vascular regrowth in OIR as assessed by a new method of quantification. Investigative Ophthalmology & Visual Science, 2006. 47(5): p. 2125-34.
46. Friedlander, M., et al., Progenitor cells and retinal angiogenesis. Angiogenesis, 2007. 10(2): p. 89-101.
47. Lutty, G. A., et al., Proceedings of the Third International Symposium on Retinopathy of Prematurity: an update on ROP from the lab to the nursery (November 2003, Anaheim, Calif.). Mol Vis, 2006. 12: p. 532-80.
48. Otani, A., et al., Rescue of retinal degeneration by intravitreally injected adult bone marrow-derived lineage-negative hematopoietic stem cells. J Clin Invest, 2004. 114(6): p. 765-74.
49. Otani, A., et al., Bone marrow-derived stem cells target retinal astrocytes and can promote or inhibit retinal angiogenesis. Nat Med, 2002. 8(9): p. 1004-10.
50. Ritter, M. R., et al., Myeloid progenitors differentiate into microglia and promote vascular repair in a model of ischemic retinopathy. J Clin Invest, 2006. 116(12): p. 3266-76.
51. Grant, M. B., et al., Adult hematopoietic stem cells provide functional hemangioblast activity during retinal neovascularization. Nat Med, 2002. 8(6): p. 607-12.
52. Caballero, S., et al., Ischemic vascular damage can be repaired by healthy, but not diabetic, endothelial progenitor cells. Diabetes, 2007. 56(4): p. 960-7.
53. Inoue, Y., et al., Subretinal transplantation of bone marrow mesenchymal stem cells delays retinal degeneration in the RCS rat model of retinal degeneration. Exp Eye Res, 2007. 85(2): p. 234-41.
54. Mead, B., et al., Intravitreally transplanted dental pulp stem cells promote neuroprotection and axon regeneration of retinal ganglion cells after optic nerve injury. Invest Ophthalmol Vis Sci, 2013. 54(12): p. 7544-56.
55. Espinosa-Heidmann, D. G., et al., Bone marrow-derived progenitor cells contribute to experimental choroidal neovascularization. Invest Ophthalmol Vis Sci, 2003. 44(11): p. 4914-9.
56. Rehman, J., et al., Secretion of angiogenic and antiapoptotic factors by human adipose stromal cells. Circulation, 2004. 109(10): p. 1292-8.
57. Sieczkiewicz, G. J. and I. M. Herman, TGF-beta 1 signaling controls retinal pericyte contractile protein expression. Microvasc Res, 2003. 66(3): p. 190-6.
58. Papetti, M., et al., FGF-2 antagonizes the TGF-beta1-mediated induction of pericyte alpha-smooth muscle actin expression: a role for myf-5 and Smad-mediated signaling pathways. Invest Ophthalmol Vis Sci, 2003. 44(11): p. 4994-5005.
59. Sieving, P. A., et al., Ciliary neurotrophic factor (CNTF) for human retinal degeneration: phase I trial of CNTF delivered by encapsulated cell intraocular implants. Proc Natl Acad Sci USA, 2006. 103(10): p. 3896-901.
60. Kauper, K., et al., Two-year intraocular delivery of ciliary neurotrophic factor by encapsulated cell technology implants in patients with chronic retinal degenerative diseases. Invest Ophthalmol Vis Sci, 2012. 53(12): p. 7484-91.
61. Fischer, M. D., et al., Successful subretinal delivery and monitoring of MicroBeads in mice. PLoS One, 2013. 8(1): p. e55173.
62. Ricker, L. J., et al., Chemokine levels in subretinal fluid obtained during scleral buckling surgery after rhegmatogenous retinal detachment. Invest Ophthalmol Vis Sci, 2010. 51(8): p. 4143-50.
63. Bai, Y., et al., Effects of semaphorin 3A on retinal pigment epithelial cell activity. Invest Ophthalmol Vis Sci, 2013. 54(10): p. 6628-38.
64. Zhang, R., et al., Neuroprotective effect of intravitreal cell-based glucagon-like peptide-1 production in the optic nerve crush model. Acta Ophthalmol, 2011. 89(4): p. e320-6.
65. Zhang, R., et al., Intravitreal cell-based production of glucagon-like peptide-1. Retina, 2011. 31(4): p. 785-9.
66. Lee, M., et al., Drug carrier systems based on collagen-alginate composite structures for improving the performance of GDNF-secreting HEK293 cells. Biomaterials, 2009. 30(6): p. 1214-21.
67. Simonsen, J. L., et al., Telomerase expression extends the proliferative life-span and maintains the osteogenic potential of human bone marrow stromal cells. Nat Biotechnol, 2002. 20(6): p. 592-6.
68. Franco, M., et al., Pericytes promote endothelial cell survival through induction of autocrine VEGF-A signaling and Bcl-w expression. Blood, 2011. 118(10): p. 2906-17.
69. King, S. N., et al., In vitro characterization of macrophage interaction with mesenchymal stromal cell-hyaluronan hydrogel constructs. J Biomed Mater Res A, 2014. 102(3): p. 890-902.
70. Shi, Y. Y., et al., Monocyte/macrophages promote vasculogenesis in choroidal neovascularization in mice by stimulating SDF-1 expression in RPE cells. Graefes Arch Clin Exp Ophthalmol, 2011. 249(11): p. 1667-79.
71. Huang, H., et al., VEGF receptor blockade markedly reduces retinal microglia/macrophage infiltration into laser-induced CNV. PLoS One, 2013. 8(8): p. e71808.
72. Samtani, S., et al., Doxycycline-mediated inhibition of choroidal neovascularization. Invest Ophthalmol Vis Sci, 2009. 50(11): p. 5098-106.
73. Zhang, Z. X., et al., Hypoxia specific SDF-1 expression by retinal pigment epithelium initiates bone marrow-derived cells to participate in Choroidal neovascularization in a laser-induced mouse model. Curr Eye Res, 2011. 36(9): p. 838-49.
74. Canto Soler, M. V., et al., A mouse model of proliferative vitreoretinopathy induced by dispase. Exp Eye Res, 2002. 75(5): p. 491-504.
75. Sequeira et al., RETINA 31:1207-1214, 2011.
76. Cronk et al., 2015, 4:1-9, Stem Cells Translational Medicine.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

1. A method for preventing or treating a retinal disease, disorder, or injury, said method comprising administering to a subject in need thereof a pharmaceutical composition comprising an effective amount of adipose-derived stem cells, said composition optionally comprising a pharmaceutically-acceptable carrier and optionally comprising an additional therapeutic agent, thereby preventing or treating a retinal disease, disorder or injury in the subject.

2. The method of claim 1, wherein said adipose-derived stem cells are capable of differentiating into pericytes.

3. The method of claim 1, further wherein adipose-derived stem cell-conditioned medium is administered to said subject.

4. The method of claim 3, wherein said adipose-derived stem cell-conditioned medium is cell-free.

5. The method of claim 3, wherein said adipose-derived stem cell-conditioned medium is serum-free.

6. The method of claim 3, wherein said conditioned medium comprises at least one of insulin-like growth factor binding protein-2 (IGFBP-2), IGFBP-3, monocyte chemoattractant protein (MCP-1), osteopontin, and stromal cell-derived factor-1 (SDF-1).

7. (canceled)

8. (canceled)

9. The method of claim 1, wherein said cells are injected intravitreally or subretinally.

10. The method of claim 1, wherein said cells integrate into retinal vasculature.

11. The method of claim 1, wherein at least one of said cells expresses pericyte markers.

12. The method of claim 11, wherein at least one of said cells differentiates into a pericyte.

13. The method of claim 1, wherein said cells are pretreated with transforming growth factor β (TGFβ) prior to administration.

14. The method of claim 13, wherein said TGFβ is used at a concentration of about 0.1 ng/ml to about 10 ng/ml.

15. (canceled)

16. The method of claim 13, wherein said pretreatment with TGFβ enhances the pericyte phenotype of the treated adipose-derived stem cells.

17. The method of claim 1, wherein TGFβ is administered to said subject after said cells are administered.

18. (canceled)

19. (canceled)

20. The method of claim 1, wherein vascular endothelial growth factor 165b (VEGF165b) is administered after said cells are administered.

21. The method of claim 1, wherein said treatment prevents or inhibits retinal capillary dropout.

22. (canceled)

23. (canceled)

24. The method of claim 1, wherein said cells are autologous cells.

25. The method of claim 1, wherein said pharmaceutical composition comprises an effective amount of at least one angiogenic factor.

26. (canceled)

27. The method of claim 1, wherein said pharmaceutical composition comprises an effective amount of at least one growth factor or cytokine.

28. The method of claim 27, wherein said growth factor or cytokine is selected from the group consisting of VEGF, (platelet-derived growth factor) PDGF, fibroblast growth factor (FGF), tumor necrosis factor α (TNFα), interleukin-6 (IL-6), and endothelin-1.

29. The method of claim 1, wherein said method stimulates vascularization of the retina.

30-34. (canceled)

35. The method of claim 1, wherein a range of about 10,000 to about 100,000,000 cells are administered to said subject.

36. (canceled)

37. (canceled)

38. The method of claim 1, wherein said cells are administered in a volume of about 0.01 ml to about 1.0 ml.

39-41. (canceled)

42. The method of claim 1, wherein said cells are encapsulated prior to administration.

43. (canceled)

44. The method of claim 42, wherein said cells are allogeneic cells from non-diabetic subjects.

45. (canceled)

46. (canceled)

47. The method of claim 1, wherein said cells integrate into the retinal vasculature.

48. (canceled)

49. The method of claim 1, wherein said subject is human.

50. (canceled)

51. The method of claim 1, wherein said disease, disorder, or injury is selected from the group consisting of diabetic retinopathy, retinopathy, arteriosclerotic retinopathy, hypertensive retinopathy, proliferative vitreoretinopathy, retinal tears, retinal detachment, macular degeneration, age related macular degeneration, inflammatory retinal disease, retinal vasculitis, retinal fibrosis, diffuse unilateral subacute neuroretinitis, cytomegalovirus retinitis, Stargardts, Best's Disease, Usher Syndrome, papilloedema, surgery, surgical/treatment side effect, vitelliform maculopathy, retinitis pigmentosa, cone-rod dystrophy, retinal separation, retinal hypoxia, aberrant neovascularization of the retina, retinal scar formation, and retinoblastoma.

52-54. (canceled)

55. The method of claim 1, wherein said method is performed during open-globe or retinal detachment repair.

56. A method for preventing or treating a retinal disease, disorder or injury, said method comprising administering intravitreally or subretinally to a subject in need thereof a pharmaceutical composition comprising adipose-derived stem cell-conditioned medium, optionally a pharmaceutically-acceptable carrier, and optionally an additional therapeutic agent, thereby treating preventing or treating a retinal disease, disorder or injury in the subject.

57. The method of claim 56, wherein said stem cell-conditioned medium is cell-free.

58. The method of claim 56, wherein said stem cell-conditioned medium is serum-free.

59. The method of claim 57, wherein said administered conditioned medium comprises an effective amount of at least one of IGFBP-2, IGFBP-3, MCP-1, osteopontin, and SDF-1.

60. (canceled)

61. The method of claim 56, wherein said cells are pretreated with TGFβ before said medium is conditioned or said cells are treated with TGFβ while said medium is being conditioned.

62-84. (canceled)

85. The method of claim 56, further wherein adipose-derived stem cells are administered.

86-89. (canceled)

90. The method of claim 56, wherein said method is performed during open-globe or retinal detachment repair.

91. A method for preventing or treating a retinal disease, disorder, or injury, said method comprising administering to a subject in need thereof an effective amount of encapsulated adipose-derived stem cells, thereby preventing or treating a retinal disease, disorder, or injury.

92. The method of claim 91, wherein said cells are administered during open-globe or retinal detachment repair.

93. The method of claim 91, wherein said cells are encapsulated in alginate based microbeads.

94. The method of claim 91, wherein said encapsulated cells are administered subretinally or intravitreally.

95-98. (canceled)

99. The method of claim 91, wherein said cells remain viable for at least one month.

100. (canceled)

101. (canceled)

102. The method of claim 91, wherein said disease, disorder, or injury is selected from the group consisting of diabetic retinopathy, retinopathy, arteriosclerotic retinopathy, hypertensive retinopathy, proliferative vitreoretinopathy, retinal tears, retinal detachment, macular degeneration, age related macular degeneration, inflammatory retinal disease, retinal vasculitis, retinal fibrosis, diffuse unilateral subacute neuroretinitis, cytomegalovirus retinitis, Stargardts, Best's Disease, Usher Syndrome, papilloedema, surgery, surgical/treatment side effect, vitelliform maculopathy, retinitis pigmentosa, cone-rod dystrophy, retinal separation, retinal hypoxia, aberrant neovascularization of the retina, retinal scar formation, and retinoblastoma.

103. (canceled)
